(19) United States
(12) Patent Application Publication
(10) Pub. No.: US 2015/0010475 A1
(43) Pub. Date: Jan. 8, 2015

(54) CRLF-2 BINDING PEPTIDES, PROTOCELLS AND VIRAL-LIKE PARTICLES USEFUL IN THE TREATMENT OF CANCER, INCLUDING ACUTE LYMPHOBLASTIC LEUKEMIA (ALL)

(71) Applicants: STC. UNM, Albuquerque, NM (US); SANDIA CORPORATION, ALBUQUERQUE, NM (US)

(72) Inventors: C. Jeffrey Brinker, Albuquerque, NM (US); David S. Peabody, Albuquerque, NM (US); Walker Kip Wharton, Corrales, NM (US); Bryce Chackerian, Albuquerque, NM (US); Carlee Erin Ashley, Albuquerque, NM (US); Cheryl L. Willman, Albuquerque, NM (US); Eric C. Carnes, Albuquerque, NM (US); Katherine Epler, Albuquerque, NM (US); Robert Eric Castillo, Albuquerque, NM (US)

(73) Assignees: STC.UNM, Albuquerque (MX); SANDA CORPORATION, ALBUQUERQUE (MX)

(21) Appl. No.: 14/369,741

(22) PCT Filed: Dec. 31, 2012
(86) PCT No.: PCT/US12/72297

§ 371 Date: Jun. 30, 2014

Related U.S. Application Data
(60) Provisional application No. 61/581,915, filed on Dec. 30, 2011.

(51) Int. Cl. A61K 47/48; C12N 15/113; C12N 15/11; C07K 7/06; A61K 31/704

(52) U.S. Cl. CPC A61K 47/48246; C07K 7/06; A61K 47/48815; A61K 31/704; C12N 15/111; C12N 15/113
USPC 424/9.6; 530/329; 424/450; 514/34; 514/44 R; 514/44 A

(57) ABSTRACT

The present invention relates to the use of which are attached or anchored phospholipid biolayers further modified by CRLF-2 and CD 19 binding peptides which may be used for delivering pharmaceutical cargos, to cells expressing CRLF-2 and CD 19, thereby treating cancer, in particular, acute lymphoblastic leukemia (ALL), including (B-precursor acute lymphoblastic leukemia (B-ALL). Novel CRLF-2 binding peptides and CLRF-2 and CD19-binding viral-like particles (VLPs) useful in the treatment of cancer, including ALL are also provided.

### RELATED APPLICATIONS

This application claims the benefit of priority of U.S. Provisional Patent Application Ser. No. 61/581,915, filed Dec. 30, 2011, and entitled “CRLF-2 Binding Peptides and CRLF-2-Targeted VLPs for Leukemia Therapy”, the complete contents of which provisional application is incorporated by reference herein.

### GOVERNMENT SUPPORT

This invention was made with government support under the NIH/Roadmap for Medical Research under grant PHS 2 PN2 EY016570B; NCI Cancer Nanotechnology Platform Partnership grant 1U01CA151792-01; the Air Force Office of Scientific Research grant 9550-10-1-0054; the U.S. Department of Energy, Office of Basic Energy Sciences, Division of Materials Sciences and Engineering; the Sandia National Laboratories' Laboratory Directed Research and Development (LDRD) program; the President Harry S. Truman Fellowship in National Security Science and Engineering at Sandia National Laboratories (C.E.A.). Accordingly, the United States has certain rights in the invention.

### FIELD OF THE INVENTION

The present invention relates to the use of which are attached or anchored phospholipid biolayers further modified by CRLF-2 and CD 19 binding peptides which may be used for delivering pharmaceutical cargos, to cells expressing CRLF-2 and CD19, thereby treating acute lymphoblastic leukemia (B-precursor acute lymphoblastic leukemia (ALL)). Novel CRLF-2 and CD19-binding viral-like particles (VLPs) useful in the treatment of ALL are also provided.

The present invention also relates to the specific oligopeptides which bind CRLF-2 CD 19 and can be used in numerous applications (therapeutic, diagnostic and the like) to treat disease states and/or conditions which are modulated through or occur in a cell which expresses CRLF-2 or CD 19. In the present invention, protocells which comprise on their surface a phospholipid bilayer and at least one CRLF-2 or CD19 binding peptide which binds to CRLF-2 or CD19 on a cell to which the protocell binds, and through endocytosis or other mechanism, the contents of the protocell is released into the targeted cell resulting in apoptosis or other cellular degradation and/or inhibition to effect an intended therapeutic result.

### BACKGROUND OF THE INVENTION

Acute lymphoblastic leukemia (“ALL”, also referred to as “childhood leukemia, of which B-precursor acute lymphoblastic leukemia (B-ALL) is the most common form is a disease characterized by the uncontrolled proliferation of malignant lymphocytes leading to the suppression of normal hematopoiesis, is the most frequently diagnosed cancer in children. Current therapies result in the induction of long term remission in 80% of pediatric ALL patients. However, death from relapsed ALL remains the second leading cause of mortality in children (surpassed only by deaths caused by accidents). In addition, children who enter remission suffer from significant life altering short- and long-term complications due to the side effects of the cytotoxic therapies. Therefore new generations of therapies are required both to enhance survival and improve quality of life in pediatric ALL patients. R. Harvey, C. Willman et el., Blood 2010 have shown that preferential expression of CRLF2 surface markers is associated specific cohorts of pediatric ALL with “poor outcome”.

The delivery of cancer therapeutic agents sequestered in nanoparticles has the potential to bypass many severe problems associated with systemic drug administration. ^{1,2} Encapsulation allows treatment with compounds that are poorly soluble and/or unstable in physiological solutions, as well as those that are rapidly metabolized or cleared when administered as free drugs. Conjugation of the particle with a targeting moiety that recognizes an antigen over-expressed on the surface of a tumor cell results in a series of additional benefits, including the limitation of damage to normal cells and a marked dose escalation that results from the localized release of highly concentrated drugs at the site of a tumor or within a cancer cell.

However, the therapeutic potential of many classes of macromolecules, especially nucleic acids and proteins, is severely limited because of degradation by plasma enzymes or an induction of an immune response following systemic administration. In addition, cellular uptake is typically restricted due to issues with either size or charge. The ability to package these molecules within particles overcomes such impediments and allows evaluation of the therapeutic efficacy of a large number of agents not presently available for clinical applications.

For example, the therapeutic potential of numerous anti-cancer and other therapeutic agents, including small and macromolecules, including traditional small molecule anticancer agents, as well as macromolecular compounds such as small interfering RNAs (siRNAs, which interfere with/silence expression of various cyclins in the cell (e.g., one or more of cyclin A2, cyclin B1, cyclin D1 or cyclin E1, among others) and protein toxins is severely limited by the availability of delivery platforms that prevent degradation and non-specific interactions during circulation but promote uptake and intracellular trafficking in targeted cells.

Despite tremendous advances, two primary challenges remain for the successful treatment of pediatric ALL. With the intensity of therapy now tailored to a child's relapse risk, nearly 80% of children survive. Yet to achieve these levels of cure, children are exposed to very intensive systemic chemotherapeutic regimens which are frequently associated with significant toxicities and serious short and long-term side effects. Thus, more targeted, less toxic treatments for ALL are needed. Secondly, 25% of children still relapse despite receiving intensive therapy and ALL remains the leading cause of cancer death in children; this is particularly true for the 30% of patients with high-risk forms of disease. More effective treatments for high-risk ALL are therefore required.

### SUMMARY OF THE INVENTION

The inventors have identified subtypes of ALL patients who have extremely poor outcomes (<25% EFS). One such group is characterized by genomic rearrangements of CRLF2 leading to markedly elevated (upregulated) levels of CRLF2 (the TSLP receptor) expression on leukemic blasts, making CRLF2 an attractive therapeutic target in high-risk ALL. CRLF2 rearrangements are frequently associated with activating mutations in the JAK kinase, deletion of IKZF1/IKAROS and other genes, Hispanic/American Indian race and ethnicity, and a very poor outcome.6-10 While virtually all ALL cases with CRLF2 genomic rearrangements have an “activated tyrosine kinase” gene expression profile, only half have JAK family mutations. Our ongoing transcriptomic sequencing studies in the NCI TARGET Project have revealed that the remaining CRLF2-expressing cases have other activating novel translocations or genomic rearrangements (PDGFR, EPOR, JAK, and ABL).

The inventors recently described a novel and remarkably versatile nanoparticle, termed a protocell (see FIG. 1), which synergistically combines features of both mesoporous silica particles and liposomes to exhibit many features of an ideal targeted therapeutic delivery platform.

The protocells are formed via fusion of liposomes to porous silica nanoparticles. The high pore volume and surface area of the spherical nanoporous silica core allow high-capacity encapsulation of a spectrum of cargos. The surrounding lipid bilayer, whose composition can be modified for specific biological applications, serves as a modular, reconfigurable scaffold, allowing the attachment of a variety of molecules that provide cell-specific targeting and controlled intracellular trafficking. Generally, our protocells target CRLF-2 and/or CD19 and have a 30- to 100-fold greater capacity for anticancer agents including siRNA than corresponding liposomes and are markedly more stable when incubated under physiological conditions. In certain applications, these protocells are loaded with low molecular weight therapeutic agents and conjugated with a peptide that specifically recognizes hepatocarcinomas induce cytotoxicity with a 10⁶-fold improvement in efficacy compared to corresponding liposomes.

Embodiments of the present invention are directed to protocells for specific targeting of cells, in particular aspects, cancer cells which express high levels of CRLF-2 and/or CD19, especially cancer cells of acute lymphoblastic leukemia, including B-cell ALL.

In certain aspects, the present invention is directed to a cell-targeting porous protocell comprising a nanoporous silica or metal oxide core with a supported lipid bilayer, and at least one further component selected from the group consisting of
a cell targeting species consisting essentially of a CRLF-2 binding peptide as otherwise described herein;
a fusogenic peptide that promotes endosomal escape of protocells and encapsulated DNA,
other cargo comprising at least one cargo component selected from the group consisting of double stranded linear DNA or a plasmid DNA;
a drug;
an imaging agent,
small interfering RNA, small hairpin RNA, microRNA, or a mixture thereof,
wherein one of said cargo components is optionally conjugated further with a nuclear localization sequence.

In certain embodiments, protocells according to embodiments of the invention comprise a nanoporous silica core with a supported lipid bilayer; a cargo comprising at least one therapeutic agent which optionally facilitates cancer cell death such as a traditional small molecule (preferably an anticancer agent which is useful in the treatment of ALL, in particular, B-ALL), a macromolecular cargo (e.g. siRNA such as S565, S7824 and/or s10234, among others, shRNA or other micro RNA or a protein toxin such as a ricin toxin A-chain or diphtheria toxin A-chain) and/or a packaged plasmid DNA (in certain embodiments—histone packaged) disposed within the nanoporous silica core (preferably supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells according to the present invention include a targeting peptide which targets cells for therapy (e.g., cancer cells in tissue to be treated) such that binding of the protocell to the targeted cells is specific and enhanced and a fusogenic peptide that promotes endosomal escape of protocells and encapsulated DNA. Protocells according to the present invention may be used in therapy or diagnostics, more specifically to treat cancer and other diseases, including viral infections, especially including childhood acute lymphoblastic leukemia, especially include B-ALL. In other aspects of the invention, protocells use novel binding peptides (CRLF-2 binding peptides as otherwise described herein) which selectively bind to cancer tissue (including leukemia cells, liver, kidney, bone and non-small cell lung cancer cells,) for therapy and/or diagnosis of cancer, including the monitoring of cancer treatment and drug discovery.

In one aspect, protocells according to embodiments of the present invention comprise a porous nanoparticle protocell which often comprises a nanoporous silica core with a supported lipid bilayer. In this aspect of the invention, the protocell comprises a targeting peptide which is CRLF-2 receptor binding peptide as otherwise described herein, often in combination with a fusogenic peptide on the surface of the protocell. The protocell may be loaded with various therapeutic and/or diagnostic cargo, including for example, small molecules (therapeutic and/or diagnostic, especially including anticancer and/or antiviral agents (for treatment of HBV and/or HCV), macromolecules including polypeptides and nucleotides, including RNA (shRNA, siRNA and other micro RNA) or plasmid DNA which may be supercoiled and histone-packaged including a nuclear localization sequence, which may be therapeutic and/or diagnostic (including a reporter molecule such as a fluorescent peptide, including fluorescent green protein/FGP, fluorescent red protein/FRP, among others).

Additional embodiments of the present invention are directed to Virus-like particles (VLPs) as otherwise described herein which express CRLF-2 binding peptides as heterologous peptides on the surface of the VLP, such as that VLP may be used to target cancer cells and deliver therapeutic cargo in the treatment of cancer, in particular ALL, including B-ALL.

Other aspects of embodiments of the present invention are directed to pharmaceutical compositions. Pharmaceutical compositions according to the present invention comprise a population of protocells which may be the same or different and are formulated in combination with a pharmaceutically acceptable carrier, additive or excipient. The protocells may be formulated alone or in combination with another bioactive agent (such as an additional anti-cancer agent or an antiviral agent) depending upon the disease treated and the route of administration (as otherwise described herein). These compositions comprise protocells as modified for a particular purpose (e.g. therapy, including cancer therapy, or diagnostics, including the monitoring of cancer therapy). Pharmaceutical compositions comprise an effective population of protocells for a particular purpose and route of administration in combination with a pharmaceutically acceptable carrier, additive or excipient.

One aspect of the present invention is directed to the finding that protocells exhibit multiple properties that overcome many of the aforementioned limitations in effectively delivering active ingredients to treat pediatric ALL by targeting CRLF-2 and/or CD 19. Specifically, in certain embodiments of the instant invention, protocells loaded with a cocktail of anticancer agents bind to cells in a manner dependent on the presence of an appropriate targeting peptide for CRLF-2 and/or CD 19, which are expressed on leukemia cells as well as on other cancer cells and, through an endocytic pathway, promote delivery of the traditional chemotherapeutic agents, anticancer agents including siRNAs and protein toxins silencing nucleotides to the cytoplasm.

The discovery of novel ALL subtypes, together with our preliminary studies demonstrating a lack of efficacy of JAK kinase inhibitors as single agents in our xenograft models of human ALL containing CRLF2 and JAK mutations, and the observation that a large percentage of high risk B-precursor ALL samples express measurable levels of CRLF2 mRNA compared to normal B cells and respond to TSLP, leads us to hypothesize that CRLF2 is a superior target for therapy in high-risk ALL.

In order to expand the universe of potential molecular targets with a parallel increase in leukemic subtypes that are amenable to treatment, as well as to allow for simultaneous targeting with multiple classes of particles, we also describe novel protocells engineered to target molecules expressed on a wider class of ALL blasts and B cell malignancies, including CD19 and CD22.

In one embodiment, the invention provides a porous nanoparticle protocell which comprises a nanoporous silica core with a supported lipid bilayer and a peptide as described herein which targets CRLF-2 and/or CD 19. Preferably, the protocell surface comprises a fusogenic peptide. The protocell may be loaded with various therapeutic and/or diagnostic cargo, including for example, small molecules that are useful in the treatment of pediatric ALL, macromolecules including polypeptides and nucleotides, including RNA (shRNA, siRNA or other micro RNA) or plasmid DNA which may be supercoiled and histone-packaged including a nuclear localization sequence, which may be therapeutic and/or diagnostic (including a reporter molecule such as a fluorescent peptide, including fluorescent green protein/FGP, fluorescent red protein/FRP, among others).

The nanoporous silica-particle core of the protocells has a high surface area, a readily variable porosity, and a surface chemistry that is easily modified. These properties make the protocell-core amenable to high-capacity loading of many different types of cargo. The protocell's supported lipid bilayer (SLB) has an inherently low immunogenicity. Additionally, the SLB provides a fluid surface to which peptides, polymers and other molecules can be conjugated in order to facilitate targeted cellular uptake. These biophysical and biochemical properties allow for the protocell to be optimized for a specific environment and enable delivery of disparate types of cargo by a wide variety of routes.

In one embodiment, the invention provides a CRLF-2 and/or CD 19-targeting protocell comprising:

(a) a core comprising a plurality of negatively-charged, nanoporous, nanoparticulate silica cores that are optionally modified with an amine-containing silane such as N-(2-aminoethyl)-3-aminopropyltrimethoxysilane (AEPTMS) and that are interspersed with one or more anticancer agents that are useful in the treatment of ALL; and
(b) a lipid bilayer which encapsulates the core and which comprises one of more lipids selected from the group consisting of 1,2-dioleoyl-sn-glycero-3-phosphocholine (DOPC), 1,2-dipalmitoyl-sn-glycero-3-phosphocholine (DPPC), dioleylglycero triethyleneglycyl iminodiacetic acid (DOIDA), distearylglycerotriethyleneglycyl iminodiacetic acid (DSIDA), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, wherein the lipid bilayer comprises a cationic lipid and one or more zwitterionic phospholipids and contains on its surface at least one peptide as otherwise described herein that targets CRLF-2 and/or CD19 (e.g. H₂N-MTAAPVHGGHHHHHH-COOH SEQ ID NO:1 or numerous 7 mer peptides including MTAAPVH SEQ ID NO:4 as otherwise described herein).

In certain embodiments, the lipid bilayer's surface also contains an R8 peptide (e.g. RRRRRRRR SEQ ID NO:23 or as modified for crosslinking/conjugation with protocells according to the present invention H₂N-RRRRRRRRGGC-COOH SEQ ID NO:2 or equivalents thereof) and/or an endosomolytic peptide (H5WYG) (e.g. GLFHAIAHFIHGGWHGLIHGWY SEQ ID NO:24 or as modified for crosslinking/conjugation with protocells according to the present invention H₂N-GLFHAIAHFIHGGWHGLIHGWYGGGC-COOH SEQ ID NO:3 or equivalents thereof).

In certain embodiments, the one or more anticancer agents that are useful in the treatment of ALL are preferably selected from the group consisting of doxorubicin, 5-fluorouracil, cisplatin, cyclophosphamide, vincristin (oncovin), vinblastine, prednisolone, procarbazine, L-asparaginase, cytarabine, hydroxyurea, 6-mercaptopurine, methotrexate, 6-thioguanine, bleomycin, etoposide, ifosfamide, sirolomus, quercetin and mixtures thereof. Preferably, one or more of doxorubicin, 5-fluoruracil and/or cisplatin are used as anticancer agents in the present invention for the treatment of ALL.

In the embodiment of the preceding paragraph, the lipid is preferably selected from the group consisting of 1,2-dioleoyl-3-trimethylammonium-propane (18:1 DOTAP) or 1,2-dioleoyl-sn-glycero-3-phospho-(1′-rac-glycerol) (DOPG), 1,2-dioleoyl-sn-glycero-3-phosphoethanolamine (DOPE) and mixtures thereof, and the protocell has at least one of the following characteristics: a BET surface area of greater than about 600 m²/g, a pore volume fraction of between about 60% to about 70%, a multimodal pore morphology composed of pores having an average diameter of between about 20 nm to about 30 nm, surface-accessible pores interconnected by pores having an average diameter of between about 5 nm to about 15 nm. Preferably, the protocell encapsulates siRNA wherein the protocell targets CRLF-2 and/or CD19 in an amount of about 0.1 nM to about 10 μM or more, about 1 to about 500 nM, about 5 to about 100 nM, about 5 to about 25 nM, about 10 nM of siRNA per 10¹⁰ nanoparticulate silica cores.

In still another embodiment, the invention provides a CRLF-2 and/or CD 19-targeting protocell comprising:

(a) a core comprising a plurality of negatively-charged, nanoporous, nanoparticulate silica cores that are optionally modified with an amine-containing silane such as N-(2-aminoethyl)-3-aminopropyltrimethoxysilane (AEPTMS) and that are interspersed with one or more anticancer agents that are useful in the treatment of ALL; and
(b) a lipid bilayer which encapsulates the core and which comprises one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,
wherein (1) the lipid bilayer comprises a cationic lipid and one or more zwitterionic phospholipids and contains on its surface at least one peptide that targets CRLF-2 and/or CD19 (“CRLF-2 binding peptide” e.g. MTAAPVH SEQ ID NO: 4 or as modified for complexation, H₂N-MTAAPVHGGHHHHHH-COOH SEQ ID NO:1 or equivalents thereof as otherwise described herein) (2) the lipid bilayer is loaded with SP94 and an endosomolytic peptide, and (3) the protocell selectively binds to a hepatocellular carcinoma cell by targeting CRLF-2 and/or CD 19.

In a preferred embodiment of the preceding paragraph, the lipid bilayer preferably comprises DOPC/DOPE/cholesterol/PEG-2000 in an approximately 55:5:30:10 mass ratio.

In still another embodiment, the invention provides a CRLF-2 and/or CD 19-targeting protocell comprising:

(a) a core comprising a plurality of negatively-charged, nanoporous, nanoparticulate silica cores that are optionally modified with an amine-containing silane such as N-(2-aminoethyl)-3-aminopropyltrimethoxysilane (AEPTMS) and that are interspersed with one or more small hairpin RNA (shRNA) and/or small interfering RNA (siRNA), other micro RNA that are useful in the treatment of ALL; and
(b) a lipid bilayer which encapsulates the core and which comprises one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,
wherein the lipid bilayer comprises a cationic lipid and one or more zwitterionic phospholipids and contains on its surface at least one peptide that targets CRLF-2 and/or CD19 (e.g. MTAAPVH SEQ ID NO:4 or as modified, for complexing with protocells H₂N-MTAAPVHGGHHHHHH-COOH SEQ ID NO:1 or other 7mer peptides or equivalents as described and/or modified).

In certain embodiments of the protocells of the invention, the lipid bilayer comprises 1,2-dioleoyl-sn-glycero-3-phosphocholine (DOPC), 1,2-dioleoyl-sn-glycero-3-phosphoethanolamine (DOPE) a polyethylene glycol (PEG), a targeting peptide, and R8 (SEQ ID NO:23), and the mesoporous, nanoparticulate silica cores each have an average diameter of around 100 nm, an average surface area of greater than 1,000 m²/g and surface-accessible pores having an average diameter of between about 20 nm to about 25 nm, and have a siRNA load of around 1 μM per 10¹⁰ particles or greater.

The targeting peptide preferably is a peptide that binds to CRLF-2 and/or CD 19 as set forth in any of FIGS. 10-14 hereof, and most preferably consists essentially of a 7-mer peptide sequence selected from the group consisting of MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8) or equivalents thereof. More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4) or equivalents thereof. Most preferably, the protocell comprises around 0.01 to around 0.02 wt % of MTAAPVH (SEQ ID NO: 4), around 10 wt % PEG-2000 and around 0.500 wt % of R8, SEQ ID. NO: 23.

In still another aspect, the invention relates to novel viral-like particles (VLPs) that target CRLF-2 and/or CD19. Preferably, the VLPs are comprised of a coat polypeptide of the bacteriophages PP7 or MS2, wherein the coat protein is modified by insertion of heterologous peptides that target CRLF-2 and/or CD 19, and wherein the peptides that target CRLF-2 and/or CD19 are displayed on the VLP and encapsidate PP7 or MS2 mRNA. These peptides include MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8) or equivalents thereof. More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4) or equivalents thereof

In still another aspect, the invention relates to a population of viral-like particles (VLPs), each of the viral-like particles comprising a bacteriophage dimer coat polypeptide on which is displayed (in the A-B loop, or at the carboxy or amino terminus of the coat polypeptide) one or more CRLF-2 targeting peptides or alternatively, single chain, variable fragments of antibodies that target a CRLF-2 and/or CD 19 epitope, wherein the one or more viral-like particles each encapsidate (1) mRNA encoding the bacteriophage, and (2) one or more anticancer agents that are useful in the treatment of ALL, preferably selected from the group consisting of doxorubicin, 5-fluorouracil, cisplatin, cyclophosphamide, vincristin (oncovin), vinblastine, prednisolone, procarbazine, L-asparaginase, cytarabine, hydroxyurea, 6-mercaptopurine, methotrexate, 6-thioguanine, bleomycin, etoposide, ifosfamide and mixtures thereof. Preferably, one or more of doxorubicin, 5-fluoruracil and/or cisplatin is used as the anticancer agent for treatment of ALL. The bacteriophage is preferably selected from the group consisting of MS2, Qb, R17, SP, PP7, GA, M11, MX1, f4, Cb5, Cb12r, Cb23r, 7s and f2 RNA bacteriophages. Preferably, the bacteriophage is a MS2 or PP7 bacteriophage.

Pharmaceutical compositions according to the present invention comprise a population of protocells or VLPs which may be the same or different and are formulated in combination with a pharmaceutically acceptable carrier, additive or excipient. The protocells may be formulated alone or in combination with another bioactive agent (such as an additional anti-cancer agent) depending upon the route of administration (as otherwise described herein). These compositions comprise protocells or VLPs as modified for a particular purpose (e.g. therapy, including cancer therapy, or diagnostics, including the monitoring of cancer therapy). Pharmaceutical compositions comprise an effective population of protocells or VLPs for a particular purpose and route of administration in combination with a pharmaceutically acceptable carrier, additive or excipient.

In further alternative aspects, the present invention relates to methods of diagnosing cancer, including pediatric ALL, the method comprising administering a pharmaceutical composition comprising a population of protocells or VLPs which have been modified to deliver a diagnostic agent or reporter imaging agent selectively to cancer cells to identify cancer, including pediatric ALL in the patient. In this method, protocells or VLPs according to the present invention may be adapted to target cancer cells, including pediatric ALL cancer cells through the inclusion of at least one targeting peptide which binds to CRLF-2 and/or CD 19 and through the inclusion of a reporter component (including an imaging agent) of the protocell targeted to the cancer cell, may be used to identify the existence and size of cancerous tissue in a patient or subject by comparing a signal from the reporter with a standard. The standard may be obtained for example, from a population of healthy patients or patients known to have cancer, including pediatric ALL. Once diagnosed, appropriate therapy with pharmaceutical compositions according to the present invention, or alternative therapy may be implemented.

In still other aspects of the invention, the compositions according to the present invention may be used to monitor the progress of therapy of cancer, including pediatric ALL, including therapy with compositions according to the present invention. In this aspect of the invention, a composition comprising a population of protocells which are specific for cancer, including pediatric ALL cancer cell binding and include a reporter component may be administered to a patient or subject undergoing therapy such that progression of the therapy of cancer, including pediatric ALL can be monitored.

Alternative aspects of the invention relate to novel CRLF-2 and/or CD19 binding peptides as otherwise described herein, which can be used as targeting peptides on protocells of certain embodiments of the present invention, or in pharmaceutical compositions for their benefit in binding CRLF-2 and/or CD 19 protein in cancer cells, including hepatocellular cancer cells, and including pediatric ALL cancerous tissue. One embodiment of the invention relates to different mer peptides (preferably, 7 mer peptides) which show activity as novel binding peptides for CRLF-2 and/or CD19 receptors. These peptides are summarized in FIG. 3 and FIGS. 10-14 and include MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8). More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4).

Each of these peptides may be used alone or in combination with other CRLF-2 and/or CD 19 binding peptides within the above group or with a spectrum of other targeting peptides (e.g., SP94 peptides as described herein) which may assist in binding protocells or VLPs according to an embodiment of the present invention to pediatric ALL cancer cells, including hepatocellular cancer cells, ovarian cancer cells, breast cancer cells and cervical cancer cells, amongst numerous other cancer cells. These peptides may be formulated alone or in combination with other bioactive agents for purposes of providing an intended result. Pharmaceutical compositions can comprise an effective amount of at least one of the CRLF-2 and/or CD 19-binding peptides identified above, in combination with a pharmaceutically acceptable carrier, additive or excipient optionally in combination with an additional bioactive agent, which may include an anticancer agent or other bioactive agent.

Methods of treating subjects suffering from cancer, including pediatric ALL are also described.

These and other aspects of the invention are described further in the Detailed Description of the Invention. In addition, certain aspects of the present invention have been discussed in detail in U.S. patent application Ser. No. 11/895,198 (US Publication 2009/0054246), entitled “A Virus-Like Platform for Rapid Vaccine Discovery”, international patent application PCT/US2012/035529 (Publication WO2012/149376), entitled “Porous Nanoparticle-Supported Lipid Bilayers (Protocells) for Targeted Delivery and Methods of Using Same” and U.S. patent application Ser. No. 12/960,168, filed Dec. 3, 2010, entitled “Virus-Like Particles as Targeted Multifunctional Nanocarriers for Delivery of Drugs, Therapeutics, Sensors and Contrast Agents to Arbitrary Cell Types”, each of which applications is incorporated by reference in its entirety herein.

### BRIEF DESCRIPTION OF THE FIGURES

FIG. 1 illustrates how a protocell is a flexible platform for targeted delivery), as determined in the experiment(s) of Example 1. The TEM image shows that a porous nanoparticle can serve as a support for lipid bilayers, which in turn seal contents within the core.

FIG. 2 illustrates the identification of targeting peptides in accordance with the invention), as determined in the experiment(s) of Example 1.

FIG. 3 shows how flow cytometry is used to evaluate binding of individual phage clones within consensus sequences. In this case, a saturatable binding curve can be constructed, allowing for the determination of the disassociation constant (K_{d}) of phage displaying potential specific peptides that bind cells with high levels of CRLF2 expression but not parental cell lines with minimal expression), as determined in the experiment(s) of Example 1.

FIG. 4 illustrates how protocells bind to target cells with high specificity at low peptide densities due to a fluid supported bilayer), as determined in the experiment(s) of Example 1.

FIG. 5 illustrates that once a targeting peptide has been selected, human cells known to over-express CRLF-2 (MMH CALL 4) are used to evaluate the binding constants of peptide that has been cross-linked to protocell-supported lipid bilayer (SLB)), as determined in the experiment(s) of Example 1. The same targeting peptide can also be displayed on protocell SLBs featuring mixtures of lipids which form segregated domains which serve to increase the local concentration of peptide.

FIG. 6 shows that targeted protocells can become internalized within target cells (MMH CALL 4, Mutz-5 and BaF3/CRLF-2) over time), as determined in the experiment(s) of Example 1. FIG. 6 also shows that targeted peptides displayed on a fluid lipid (DOIDA) domain within a non-fluid SLB (DSPC) show high binding affinity to target cells which over-express CRF-2 (MHH Cell 4, Mutz and BaF3/CRLF-2).

FIG. 7 shows that targeted protocells can become internalized within target cells (MHH CALL4, Mutz-5 and BaF3/CRLF-2) over time), as determined in the experiment(s) of Example 1.

FIG. 8 illustrates that targeted protocells that display the R8 peptide show increased internalization kinetics), as determined in the experiment(s) of Example 1.

FIG. 9 illustrates that CRLF-2 specific protocells loaded with the chemotherapeutic agent doxorubicin (DOX) induce apoptosis of CRLF-2-positive cells (MHH CALL4) but not CRLF-2 negative cells (MOLT4), as determined in the experiment(s) of Example 1.

FIGS. 10 and 11 depict data for selections against BaF3/CRLF-2 (4° C. as determined in the experiment(s) of Example 1.

FIGS. 12 and 13 depict data for selections against BaF3/CRLF-2 (37° C.), as determined in the experiment(s) of Example 1.

FIG. 14 depicts data for selections against BaF3/CRLF-2 (37° C. with trypsin), as determined in the experiment(s) of Example 1.

FIGS. 15(1)-15(8). Flow cytometry data of targeted and non-targeted protocells to Baf3/CRLF2 and BaF3 parental cell lines, as determined in the experiment(s) of Example 1. Particles were labeled with Alexa-fluor-647 and incubated with various cell types for an hour before the samples were washed and immediately measured using a FACS Caliber Flow Cytometer. FIGS. 15(1) to 15(6) show results for MTAAPVH-targeted protocells. FIGS. 15(7) and 15(8) show GE-11 targeted protocells.

FIGS. 15(9-15(10). FIG. 15(9) shows the structure of a plasmid that expresses the MS2 coat protein single-chain dimer with a fusion of a CRLF2 targeting peptide (TDAHASV SEQ ID NO:7) at its N-terminus. FIG. 15(10) shows the results of FACS analysis, which reveals the ability of the targeted VLPs to specifically bind only the cells producing CRLF2.

FIG. 16(a). CD19 IgG1 was partially reduced via reaction with a 60-fold molar excess of TCEP for 20 minutes at room temperature. Reduced antibody was then desalted and incubated with protocells (DOPC with 30 wt % cholesterol and 10 wt % maleimide-PEG-DMPE) overnight at 4 C. Protocells were washed 3× with PBS before being added to cells. Data determined in the experiment of Example 2.

FIG. 16(b) shows that VLPs displaying anti-CD19 bind to CD19-expressing NALM6 cells, but not CD19-negative Jurkat cells (not shown). Data determined in the experiment of Example 2.

FIG. 17(a). Hierarchical Clustering Identifies 8 Cluster Groups in High Risk ALL. Hierarchical clustering using 100 genes (provided in Kang³⁹) was used to identify clusters of patients with shared patterns of gene expression. (Rows: Top 100 Probe Sets; Columns: 207 ALL Patients). Shades of red depict expression levels higher than the median while green indicates levels lower than the median. The 8 cluster groups are outlined. Cases with an MLL translocation are noted in yellow at the bottom of the figure while cases with a t(1;19)(TCF3-PBX1) are noted in bright green. Cases clustered in H2 that lacked a t(1;19)(TCF3-PBX1) are noted in dark green. The red bars note patients who relapsed. Data determined in the experiment of Example 3.

FIG. 17(b). Survival in Gene Expression Cluster Groups. Relapse-free survival is shown for the patients in Cluster 8 (Panel A), or those who express high levels of CRLF2 (Panel B) or C199 (Panel C). Red lines indicate the patients in the cluster or with high gene expression while the black lines represent those either in other cluster or with low levels of expression. Data determined in the experiment of Example 3.

FIG. 18. Binding of M13 phage displaying a CRLF2-specific peptide for BaF3-CRLF2 and BaF3 parental cells. Data determined in the experiment of Example 4.

FIG. 19. CRLF2-targeted protocell binding/internalization by CRLF2-positive cells (MUTZ5, MHHCALL4, BaF3/CRLF2) vs. controls (BaF3 parental or NALM6 cells), as determined in the experiment(s) of Example 5. A. CRLF2 targeting peptide density dependence of dissociation constant K_{d}. B. Confocal images of DOX (fluorescent red) loaded protocells (silica; white) after incubation with BaF3/CRLF2 or parental BaF3. C. Flow cytometric binding of CRLF2-targeted protocells (loaded with DOX) after binding and internalization in BaF3/CRLF2 and parental cells with varying densities of octa-arginine (R8), which promotes internalization.

FIG. 20 illustrates uptake of CRLF2-Targeted Protocells in Established ALL Cell Lines (Mutz-5 and MHH CALL4) with High CRLF2 Cell Surface Expression vs. Controls (NALM-6). Left Panels: Cell lines incubated with non-targeted protocells (top panels), then: 1) CRLF2-targeted protocells after one hour at 4° C., 2) CRLF2-targeted protocells after one hour at 37° C., and 3) CRLF2-targeted protocells after 24 hours at 37° C., imaged using hyperspectral confocal fluorescence microscopy which detects the encapsidated drug cargo (fluorescent doxorubicin (red), DOX in each panel) as well as fluorescent silica cores (white). Right Panels: Flow cytometric assays demonstrating intracellular uptake of both DOX and the CRLF2-targeted protocells in 2-color flow cytometric assays in CRLF2-expressing cell lines (Mutz-5, MHH CALL4), but not control cells (NALM-6). Data determined in the experiment(s) of Example 5.

FIG. 21 depicts fluorescent images of CRLF-2-expressing ALL cells (MHH CALL4) and control cells (NALM6) that were continually exposed to 75 nM of doxorubicin encapsidated within CRLF-2-targeted, R8-modified protocells) for 48 hours at 37° C.), as determined in the experiment(s) of Example 5. MHH CALL4 and not NALM6 cells demonstrate doxorubicin uptake and apoptosis (annexin V) ALL-targeted protocell specificity and toxicity.

FIG. 22 illustrates live animal biophotonic imaging of dye-loaded protocells (red, left panel), from 0 to 8 mg, 8 hours after injection), as determined in the experiment(s) of Example 5. These non-targeted protocells initially distributed widely (protocells in the bladder are seen in the 2 mg mouse) and later concentrated in the liver. Detection of dye-loaded protocells (red, middle panel) in ALL-bearing mice (green, right panel). The CBG ALL cells in the same mice are depicted in the middle and right panels.

FIG. 23 illustrates the creation of CRLF2 (+) and (−) ALL Models. A) Using lentiviral-mediated gene transfer, REH parental cells were modified to express CBG and GFP plus/minus the CRLF2 gene), as determined in the experiment(s) of Example 5. Flow cytometry demonstrates uniform expression of GFP and two separate limiting dilution clones (3 and 4) over-expressing CRLF2. B) Primary human ALL samples (see text) with or without CRLF2 & JAK mutations. CRLF2 expression is detected by flow cytometry. Below each set of histograms is in vivo imaging of the primary or REH sample. Pseudocolor heat maps indicate presence of ALL on Day 20 (JH331, JL491, NL482b) or Day 3 (REH). No peripheral blasts are detectable at these times.

FIG. 24 illustrates the non-specific toxicity of protocells is a function of the charge of lipids employed in the SLB. (A) The degree to which ‘empty’ SP94-targeted protocells and liposomes, as well as nanoporous silica cores induce oxidative stress and subsequent cell death in Hep3B was determined using MitoSOX Red, a mitochondrial superoxide indicator that fluoresces in the presence of superoxide anions, and propidium iodide, respectively. Positively- and negatively-charged polystyrene nanoparticles (amine-PS and carboxyl-PS, respectively) were employed as positive controls, while Hep3B exposed to 10 mM of the antioxidant, N-acetylcysteine (NAC), was used as a negative control. All error bars represent 95% confidence intervals (1.96σ) for n=3. (B) Confocal fluorescence microscopy of Hep3B cells exposed to DOPC or DOTAP protocells for 24 hours at 37° C. prior to being stained with either MitoSOX Red or Alexa Fluor® 488-labeled annexin V (green) and propidium iodide (red). Nuclei are stained with DAPI. Scale bars=20 μm. Data determined in the experiment of Example 5.

FIG. 25 illustrates MS2 SP94 serum dilution versus OD-405, as determined in the experiment(s) of Example 5.

FIG. 26 illustrates that combinations of peptides can be used to direct targeting and internalization for non-internalized receptors, as determined in the experiment(s) of Example 6.

FIGS. 27-29 depict the effect of 4 mg of fluorescently labeled protocells in a murine luminescent leukemia model, as determined in the experiment of Example 7.

FIG. 30 illustrates the MS2 VLP affinity selection process, as described in the experiment of Example 8.

FIG. 31 depicts binding of M13 phage displaying a CRLF2-specific peptide for BaF3-CRLF2 and BaF3 parental cells, as determined in the experiment(s) of Example 9.

FIG. 32 depicts VLPs displaying anti-CD19 binding/CD19-expressing NALM6 cells, but not CD 19-negative Jurkat cells (not shown), as determined in the experiment(s) of Example 9.

FIG. 33 illustrates protocell binding, internalization and delivery, as determined in the experiment(s) of Example 9.

FIG. 34 illustrates that protocells modified with only six SP94 peptides per particle exhibit a 10,000-fold greater affinity for Hep3B than for normal hepatocytes, and other control cells, as determined in the experiment(s) of Example 9.

FIG. 35 illustrates that CRLF2-targeted protocells were demonstrated to possess a 1,000-fold higher affinity for engineered BaF3-CRLF2 cells expressing high levels of CRLF2, as determined in the experiment(s) of Example 9.

FIG. 36 illustrates uptake of CRLF2-targeted protocells in established ALL cell lines (Mutz-5 and MHH CALL4) with high CRLF2 cell surface expression versus CRLF2-negative ALL cells (NALM-6), as determined in the experiment(s) of Example 9.

FIG. 37 illustrates fluorescent images of CRLF2-expressing ALL cells (MHH CALL4) and CRLF2-negative controls (NALM6) that were continually exposed to 75 nM of doxorubicin encapsidated with CRLF2-targeted, R8-modified protocells for 48 hours at 37° C.), as determined in the experiment(s) of Example 9.

FIG. 38 illustrates the impact of mTOR inhibition on four high-risk ALL xenograft models representative of four different CRLF2/JAK genotypes), as determined in the experiment(s) of Example 9.

FIG. 39 illustrates fluorescently tagged NALM6 cells that were transduced with a retrovirus directing the expression of ectopic human CRLF2 and stable clones with a 10-fold increase in surface expression), as determined in the experiment(s) of Example 10.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present invention. Exemplary bioactive agents include the compounds according to the present invention which are used to treat pediatric ALL or a disease state or condition which occurs secondary to pediatric ALL and may include antiviral agents as well as other compounds or agents which are otherwise described herein.

The terms “treat”, “treating”, and “treatment”, are used synonymously to refer to any action providing a benefit to a patient at risk for or afflicted with cancer, preferably pediatric ALL, including improvement in the condition through lessening, inhibition, suppression or elimination of at least one symptom, delay in progression of pediatric ALL, prevention, delay in or inhibition of the likelihood of the onset of pediatric ALL, etc. In the case of viral infections associate with pediatric ALL, these terms also apply to viral infections and preferably include, in certain particularly favorable embodiments the eradication or elimination (as provided by limits of diagnostics) of the virus which is the causative agent of the infection.

Treatment, as used herein, encompasses both prophylactic and therapeutic treatment of cancer, principally including pediatric ALL, but also of other disease states associated with pediatric ALL including viral infections. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present invention is effective to decrease the severity of the disease or lengthen the lifespan of the mammal so afflicted, as in the case of cancer, or inhibit or even eliminate the causative agent of the disease, as in the case of viral co-infections.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The term “protocell” is used to describe a porous nanoparticle which is made of a material comprising, e.g. silica, polystyrene, alumina, titania, zirconia, or generally metal oxides, organometallates, organosilicates or mixtures thereof.

In certain embodiments, the porous particle core may be hydrophilic and can be further treated to provide a more hydrophilic surface in order to influence pharmacological result in a particular treatment modality. For example, mesoporous silica particles according to the present invention can be further treated with, for example, ammonium hydroxide or other bases and hydrogen peroxide to provide significant hydrophilicity. The use of amine containing silanes such as 3-[2-(2-aminoethylamino)ethylamino]propyltrimethoxysilane (AEPTMS), among others, may be used to produce negatively charged cores which can markedly influence the cargo loading of the particles. Other agents may be used to produce positively charged cores to influence in the cargo in other instances, depending upon the physicochemical characteristics of the cargo.

Nanoparticles according to the present invention comprise a lipid bilayer which coats its surface to form a structure referred to as a protocell. While numerous lipids and phospholipids may be used to provide a lipid bilayer for use in the present invention, in certain preferred embodiments, the lipid bilayer comprises a phospholipid selected from the group consisting of phosphatidyl choline, 1,2-Dioleoyl-3-Trimethylammonium-propane (DOTAP), 1,2-Dioleoyl-sn-Glycero-3-Phosphocholine (DOPC), 1,2-dioleoyl-sn-glycero-3-phosphoethanolamine-N-[methoxy(polyethylene glycol)-2000, 1,2-dioleoyl-sn-glycero-3-phosphoethanolamine (DOPE) or mixtures thereof. In addition to a phospholipid, including the specific phospholipids as otherwise described herein, the lipid bilayer may also comprise cholesterol (for structural integrity of the lipid bilayer) as well as polyethylene glycol lubricants/solvents (e.g. PEG 2000, etc.) to provide flexibility to the lipid bilayer. In addition to fusing a single phospholipid bilayer, multiple bilayers with opposite charges may be fused onto the porous particles in order to further influence cargo loading, sealing and release of the particle contents in a biological system.

In certain embodiments, the lipid bilayer can be prepared, for example, by extrusion of hydrated lipid films through a filter of varying pore size (e.g., 50, 100, 200 nm) to provide filtered lipid bilayer films, which can be fused with the porous particle cores, for example, by pipette mixing or other standard method.

In various embodiments, the protocell (nanoparticle to which a lipid bilayer covers or is otherwise fused to the particle) can be loaded with and seal macromolecules (shRNAs, siRNAs, other micro RNA and polypeptide toxins) as otherwise described herein, thus creating a loaded protocell useful for cargo delivery across the cell membrane

In preferred aspects of the present invention, the protocells provide a targeted delivery through conjugation of certain targeting peptides onto the protocell surface, preferably by conjugation to the lipid bilayer surface. These peptides include SP94 and H5WYG peptides which may be synthesized with C-terminal cysteine residues and conjugated to one or more of the phospholipids (especially, DOPE, which contains a phosphoethanolamine group) which comprise the lipid bilayer or conjugated to the phospholipids using one or more conjugating agents.

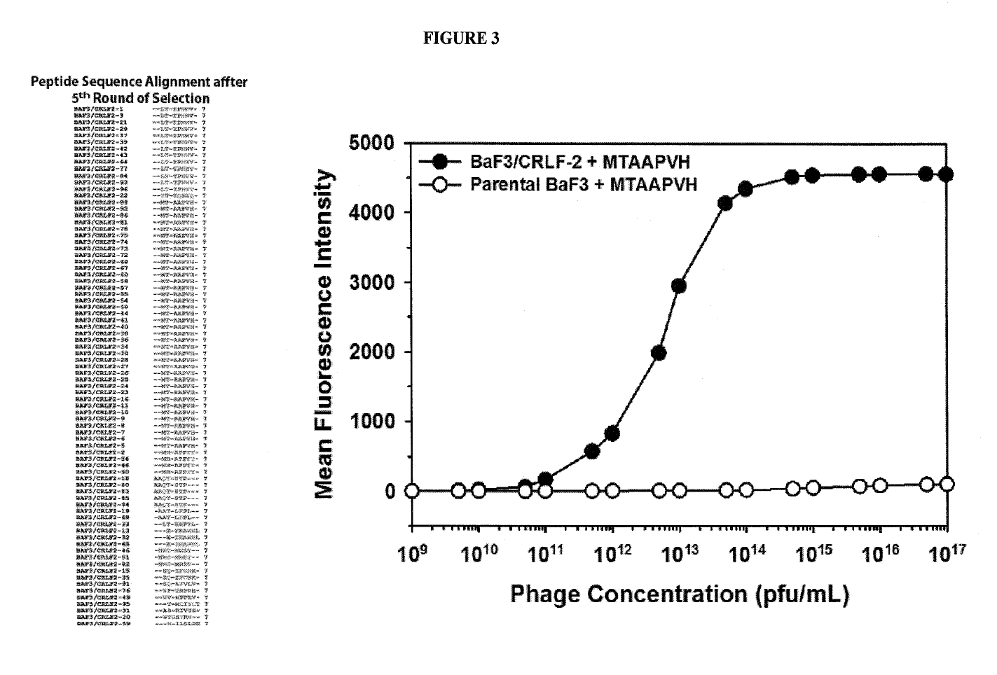

The term “targeting peptide” is used to describe a preferred targeting active species which is a peptide of a particular sequence (preferably a 7mer as otherwise described herein, which binds to a CRLF-2 receptor or other polypeptide in cancer cells and allows the targeting of protocells according to the present invention to particular cells which express a peptide (be it a receptor or other functional polypeptide) to which the targeting peptide binds. In the present invention, exemplary targeting peptides include, for example, those which appear in FIGS. 3, and 10-14 hereof, and preferably include the following targeting peptides: MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8). More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4).

Targeting peptides used herein are generally covalently anchored/complexed to the phospholipic bilayer of VLPs as otherwise described herein by conjugation through a crosslinking agent or by complexing an appropriately modified peptide with an oligopeptide such as hexameric histidine which can bind to copper and/or nickel complexes of the phospholipid bilayer. Conjugation of peptides to phospholipids represents a preferred approach for attaching targeting peptides to protocells according to the present invention. using crosslinking agents as otherwise described herein.

Other targeting peptides are known in the art. Targeting peptides may be complexed or preferably, covalently linked to the lipid bilayer through use of a crosslinking agent as otherwise described herein.

The term “crosslinking agent” is used to describe a bifunctional compound of varying length containing two different functional groups which may be used to covalently link various components according to the present invention to each other. Crosslinking agents according to the present invention may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells. There are a large number of crosslinking agents which may be used in the present invention, many commercially available or available in the literature. Preferred crosslinking agents for use in the present invention include, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol]ester (SM(PEG)₂₄), and succinimidyl 6-[3′-(2-pyridyldithio)-propionamido]hexanoate (LC-SPDP), among others. Other crosslinking agents include, for example, AMAS, BMPS, GMBS, sulfo-GMBS, MBS, sulfo-MBS, SMCC, sulfo-SMCC, EMCS, sulfo-EMCS, SMPB, sulfo-SMPB, SMPH, LC-SMCC, Sulfo-KMUS, SM(PEG)nNHS-PEG-Maleimide Crosslinkers, SPDP, LC-SPDP, sulfo-LC-SPDP, SMPT, sulfo-LC-SMPT, SIA, SBAP, SIAB, or SIAB. These crosslinkers are well known in the art. In some instances, it may be advantageous to use crosslinkers which are cleavable via reduction or at lower pH (selective for cancer cells). Using cleavable crosslinkers helps to liberate cytotoxic agents in the cytosol of target cells, e.g., cancer cells. Exemplary cleavable crosslinking agents for use herein include SPDP, LC-SPDP, sulfo-LC-SPDP, SMPT and sulf-LC-SMPT, among others.

The terms “fusogenic peptide” and “endosomolytic peptide” are used synonymously to describe a peptide which is optionally and preferred crosslinked onto the lipid bilayer surface of the protocells or incorporated into the VLP according to the present invention. Fusogenic peptides are incorporated onto protocells or into VLPS in order to facilitate or assist escape from endosomal bodies and to facilitate the introduction of protocells or VLPs into targeted cells to effect an intended result (therapeutic and/or diagnostic as otherwise described herein). Representative and preferred fusogenic peptides for use in protocells according to the present invention include H5WYG peptide, GLFHAIAHFIHGGWHGLIHGWY (SEQ ID NO:24) modified for conjugation/crosslinking as H₂N-GLFHAIAHFIHGGWHGLIHGWYGGC-COOH (SEQ ID. NO: 3) or an 8 mer polyarginine (H₂N-RRRRRRRR-COOH, SEQ ID NO:23), modified for conjugation/crosslinking as RRRRRRRRGGC, SEQ ID NO:2, among others known in the art.

As used herein, unless otherwise specified, the term “protocell” refers to a nanostructure having a porous particle and a lipid bilayer surrounding the porous particle. The protocell can mimic bioactive cells (or real cells) that have a supported lipid bilayer membrane. For example, the porous particle can be made of a material including polystyrene, silica, alumina, titania, zirconia, etc. In embodiments, the porous particle 110 can have a controllable average pore size ranging from about 2 nm to about 30 nm, and an average porosity ranging from about 10% to about 70%, for example, ranging from about 25% to about 50%. The porous particle can have an average particle size ranging from about 30 nm to about 3000 nm.

The porous particle, such as porous silica particles, can be surface charged. For example, the surface charge of the porous silica particles can switch from negative to positive at neutral pHs by using amine-modified silane precursors and controlling the percentage of amine groups within the porous silica particles. For example, the porous silica particles can have a composition of about 5% to about 50% amine, such as about 10% to about 50% amine, or about 5% to about 30% amine by weight; and the amine-modified silane precursors can include, for example,

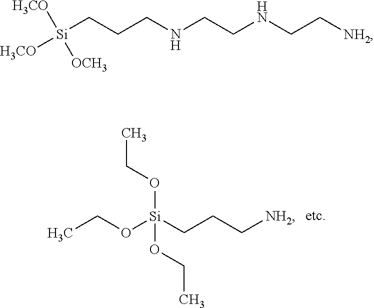

The porous silica particles can be formed by, for example, mixing water, HCl, ethanol, cetyltrimethylammonium bromide (CTAB), and tetraethyl orthosilicate (TEOS), as disclosed in a related International Patent Application No. PCT/US10/20096, entitled “Porous Nanoparticle Supported Lipid Bilayer Nanostructures,” which is hereby incorporated by reference in its entirety.

Porous nanoparticulates used in protocells of the invention include mesoporous silica nanoparticles and core-shell nanoparticles.

The porous nanoparticulates can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle is used for the preferred protocells and is surrounded by a supported lipid or polymer bilayer or multilayer. Various embodiments according to the present invention provide nanostructures and methods for constructing and using the nanostructures and providing protocells according to the present invention. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from Melorium Technologies, Rochester, N.Y. SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells which are useful in the present invention. Protocells according to the present invention may be readily prepared, including protocells comprising lipids which are fused to the surface of the silica nanoparticle. See, for example, Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Preferred protocells for use in the present invention are prepared according to the procedures which are presented in Ashley, et al., Nature Materials, 2011, May; 10(5):389-97, Lu, et al., Nature, 398, 223-226 (1999), Caroll, et al., Langmuir, 25, 13540-13544 (2009), and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall.

In certain embodiments, the porous nanoparticulates are comprised of one or more compositions selected from the group consisting of silica, a biodegradable polymer, a solgel, a metal and a metal oxide.

In an embodiment of the present invention, the nanostructures include a core-shell structure which comprises a porous particle core surrounded by a shell of lipid preferably a bilayer, but possibly a monolayer or multilayer (see Liu, et al., JACS, 2009, Id). The porous particle core can include, for example, a porous nanoparticle made of an inorganic and/or organic material as set forth above surrounded by a lipid bilayer. In the present invention, these lipid bilayer surrounded nanostructures are referred to as “protocells” or “functional protocells,” since they have a supported lipid bilayer membrane structure. In embodiments according to the present invention, the porous particle core of the protocells can be loaded with various desired species (“cargo”), including small molecules (e.g. anticancer agents as otherwise described herein), large molecules (e.g. including macromolecules such as RNA, including small interfering RNA or siRNA or small hairpin RNA or shRNA, or other micro RNA or a polypeptide which may include a polypeptide toxin such as a ricin toxin A-chain or other toxic polypeptide such as diphtheria toxin A-chain DTx, cholera toxin A-chain, among others) or a reporter polypeptide (e.g. fluorescent green protein, among others) or semiconductor quantum dots, or metallic nanoparticles, or metal oxide nanoparticles or combinations thereof. In certain preferred aspects of the invention, the protocells are loaded with super-coiled plasmid DNA, which can be used to deliver a therapeutic and/or diagnostic peptide(s) or a small hairpin RNA/shRNA, small interfering RNA/siRNA or other micro RNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, various cyclins as described hereinabove, among numerous others, and induce growth arrest and apoptosis of cancer cells).

In certain embodiments, the cargo components can include, but are not limited to, chemical small molecules (especially anticancer agents and antiviral agents, nucleic acids (DNA and RNA, including siRNA, shRNA, other micro RNA and plasmids which, after delivery to a cell, express one or more polypeptides or RNA molecules), such as for a particular purpose, such as a therapeutic application or a diagnostic application as otherwise disclosed herein.

In embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

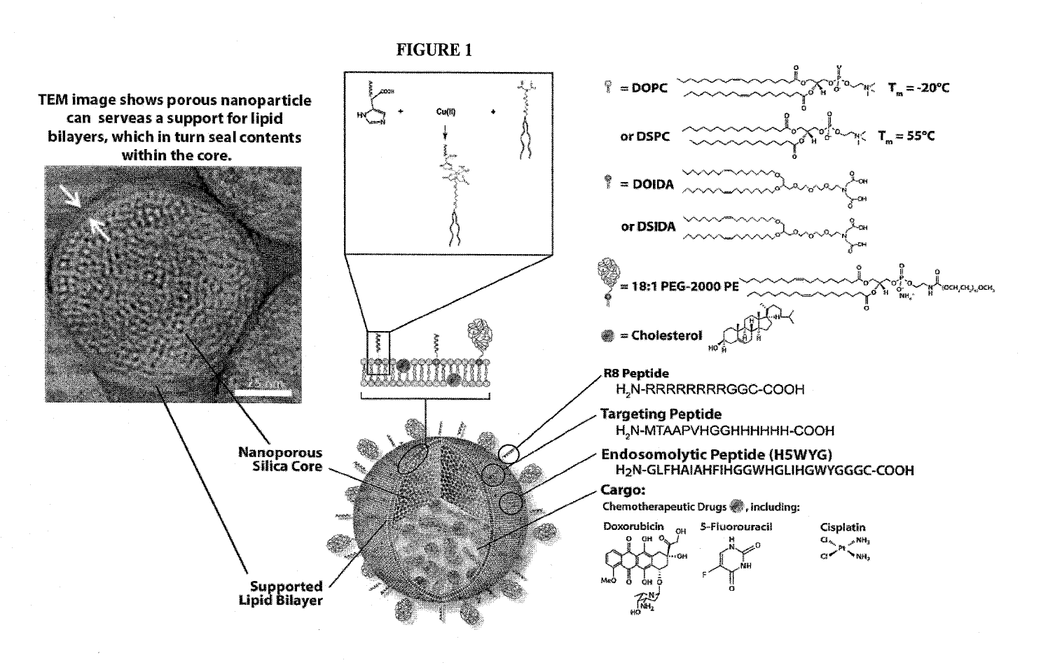

The protocells particle size distribution, according to the present invention, depending on the application, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. See FIG. 1, attached. Polydisperse populations can be sized into monodisperse populations. All of these are suitable for protocell formation. In the present invention, preferred protocells are preferably no more than about 500 nm in diameter, preferably no more than about 200 nm in diameter in order to afford delivery to a patient or subject and produce an intended therapeutic effect.

In one embodiment, the present invention is directed to high surface area (i.e., greater than about 600 m²/g, preferably about 600 to about 1,000-1,250 mg²/g), preferably monodisperse spherical silica or other biocompatible material nanoparticles having diameters falling within the range of about 0.05 to 50 μm, preferably about 1,000 nm or less, more preferably about 100 nm or less, 10-20 nm in diameter, a multimodal pore morphology comprising large (about 1-100 nm, preferably about 2-50 nm, more preferably about 10-35 nm, about 20-30 nm) surface-accessible pores interconnected by smaller internal pores (about 2-20 nm, preferably about 5-15 nm, more preferably about 6-12 nm) volume, each nanoparticle comprising a lipid bilayer (preferably a phospholipid bilayer) supported by said nanoparticles (the phospholipic bilayer and silica nanoparticles together are labeled “protocells”), to which is bound at least one antigen which binds to a targeting polypeptide or protein on a cell to which the protocells are to be targeted, wherein the protocells further comprise (are loaded) with a small molecule anticancer agent and/or a macromolecule selected from the group consisting of a short hairpin RNA (shRNA), a small interfering RNA (siRNA) or a polypeptide toxin (e.g. ricin toxin A-chain or other toxic polypeptide).

The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072. In certain embodiments, protocells according to the present invention utilize nanoparticles to form protocells which are monodisperse.

In certain embodiments, protocells according to the present invention generally range in size from greater than about 8-10 nm to about 5 μm in diameter, preferably about 20-nm-3 μm in diameter, about 10 nm to about 500 nm, more preferably about 20-200-nm (including about 150 nm, which may be a mean or median diameter). As discussed above, the protocell population may be considered monodisperse or polydisperse based upon the mean or median diameter of the population of protocells. Size is very important to therapeutic and diagnostic aspects of the present invention as particles smaller than about 8-nm diameter are excreted through kidneys, and those particles larger than about 200 nm are trapped by the liver and spleen. Thus, an embodiment of the present invention focuses in smaller sized protocells for drug delivery and diagnostics in the patient or subject.

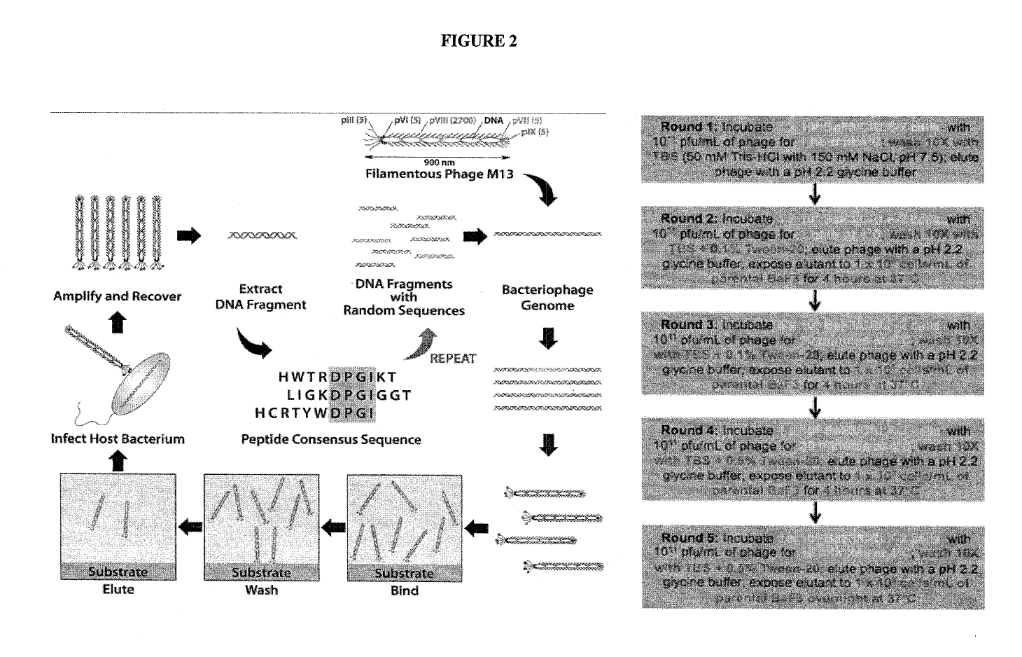

In certain embodiments, protocells according the present invention are characterized by containing mesopores, preferably pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Preferred pore sizes of mesopores range from about 2-30 nm; they can be monosized or bimodal or graded—they can be ordered or disordered (essentially randomly disposed or worm-like). See FIG. 2, attached.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. See FIG. 3, attached. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m2/g to >about 1200 m2/g. In general, the larger the pore size, the smaller the surface area. See table FIG. 2A. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells according to the present invention are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the protocell. This is generally expressed in μM per 10¹⁰ particles where we have values ranging from 2000-100 μM per 10¹⁰ particles. Preferred protocells according to the present invention exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the protocells according to one embodiment of the present invention, the surface area is mainly internal as opposed to the external geometric surface area of the nanoparticle.

The lipid bilayer supported on the porous particle according to one embodiment of the present invention has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bilayer may vary significantly in composition. Ordinarily, any lipid or polymer which is may be used in liposomes may also be used in protocells. Preferred lipids are as otherwise described herein. Particularly preferred lipid bilayers for use in protocells according to the present invention comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment of the present invention are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. Quantitative experimental evidence has shown that targeted protocells illicit only a weak immune response, because they do not support T-Cell help required for higher affinity IgG, a favorable result.

Protocells according to the present invention exhibit at least one or more a number of characteristics (depending upon the embodiment) which distinguish them from prior art protocells:
1) The protocells target CRLF-2 and/or CD 19 and in contrast to the prior art, an embodiment of the present invention specifies nanoparticles whose average size (diameter) is less than about 200-nm—this size is engineered to enable efficient cellular uptake by receptor mediated endocytosis;
2) An embodiment of the present invention targets CRLF-2 and/or CD 19 and can specify both monodisperse and/or polydisperse sizes to enable control of biodistribution.
3) An embodiment of the present invention is directed to nanoparticles that target CRLF-2 and/or CD19 and that induce receptor mediated endocytosis.
4) An embodiment of the present invention targets CRLF-2 and/or CD 19 and induces dispersion of cargo into cytoplasm through the inclusion of fusogenic or endosomolytic peptides.
5) An embodiment of the present invention targets CRLF-2 and/or CD19 and provides particles with pH triggered release of cargo.
6) An embodiment of the present invention targets CRLF-2 and/or CD19 and exhibits controlled time dependent release of cargo (via extent of thermally induced crosslinking of silica nanoparticle matrix).
7) An embodiment of the present invention targets CRLF-2 and/or CD19 and can exhibit time dependent pH triggered release.
8) An embodiment of the present invention targets CRLF-2 and/or CD19 and can contain and provide cellular delivery of complex multiple cargoes.
9) An embodiment of the present invention shows the killing of CRLF-2 and/or CD19-expressing cancer cells.
10) An embodiment of the present invention shows diagnosis of CRLF-2 and/or CD19-expressing cancer cells.
11) An embodiment of the present invention shows selective entry of target cells.
12) An embodiment of the present invention shows selective exclusion from off-target cells (selectivity).
13) An embodiment of the present invention targets CRLF-2 and/or CD 19-expressing cancer cells and shows enhanced fluidity of the supported lipid bilayer.
14) An embodiment of the present invention targets CRLF-2 and/or CD19-expressing cancer cells and exhibits sub-nanomolar and controlled binding affinity to target cells.
15) An embodiment of the present invention exhibits sub-nanomolar binding affinity to CRLF-2 and/or CD19-expressing cancer cells and also exhibits targeting ligand densities below concentrations found in the prior art.
16) An embodiment of the present invention can further distinguish the prior art with with finer levels of detail unavailable in the prior art.

The term “lipid” is used to describe the components which are used to form lipid bilayers on the surface of the nanoparticles which are used in the present invention. Various embodiments provide nanostructures which are constructed from nanoparticles which support a lipid bilayer(s). In embodiments according to the present invention, the nanostructures preferably include, for example, a core-shell structure including a porous particle core surrounded by a shell of lipid bilayer(s). The nanostructure, preferably a porous silica nanostructure as described above, supports the lipid bilayer membrane structure. In embodiments according to the invention, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20%, preferably about 5% to about 15%, about 10% by weight of the lipids which are included in the lipid bilayer.

Numerous lipids which are used in liposome delivery systems may be used to form the lipid bilayer on nanoparticles to provide protocells according to the present invention. Virtually any lipid which is used to form a liposome may be used in the lipid bilayer which surrounds the nanoparticles to form protocells according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the present invention given the fact that cholesterol may be an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly(lactic acid) (PLA), poly(glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the present invention can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence.

Mesoporous silica nanoparticles can be e.g. from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙSiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536, 569, the disclosure of such processes therein are incorporated herein by reference.

“Amine-containing silanes” include, but are not limited to, a primary amine, a secondary amine or a tertiary amine functionalized with a silicon atom, and may be a monoamine or a polyamine such as diamine. Preferably, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used.

In certain embodiments of a protocell of the invention, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments:

(a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (A9-Cis)], POPC [16:0-18:1] and DOTAP [18:1]; and wherein
(b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PT-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

In one illustrative embodiment of a protocell of the invention:

(a) include at least one anticancer agent that targets CRLF-2 and/or CD 19-expressing cancer cells and that is effective in the treatment of pediatric ALL, including B-Cell ALL;
(b) less than around 10% to around 20% of the anticancer agent is released from the porous nanoparticulates in the absence of a reactive oxygen species; and
(c) upon disruption of the lipid bilayer as a result of contact with a reactive oxygen species, the porous nanoparticulates release an amount of anticancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anticancer agent that would have been released had the lipid bilayer been lysed with 5% (w/v) Triton X-100.

One illustrative embodiment of a protocell of the invention includes at least one anticancer agent that targets CRLF-2 and/or CD 19-expressing cancer cells, is effective in the treatment of ALL and that comprises a plurality of negatively-charged, nanoporous, nanoparticulate silica cores that:

(a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof;
(b) are loaded with a shRNA, siRNA or ricin toxin A-chain or mixtures thereof; and
(c) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof; and wherein the lipid bilayer comprises a cationic lipid and one or more zwitterionic phospholipids.

Protocells of the invention can comprise a wide variety of pharmaceutically-active ingredients in addition to anticancer agents that target CRLF-2 and/or CD19-expressing cancer cells and that are effective in the treatment of cancer, including pediatric ALL.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell.

Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, “epitope” refers to an antigenic determinant of a polypeptide. An epitope could comprise 3 amino acids in a spatial conformation which is unique to the epitope. Generally an epitope consists of at least 5 such amino acids, and more usually, consists of at least 8-10 such amino acids. Methods of determining the spatial conformation of amino acids are known in the art, and include, for example, x-ray crystallography and 2-dimensional nuclear magnetic resonance.

As used herein, a “mimotope” is a peptide that mimics an authentic antigenic epitope.

As used herein, the term “coat protein(s)” refers to the protein(s) of a bacteriophage or a RNA-phage capable of being incorporated within the capsid assembly of the bacteriophage or the RNA-phage.

As used herein, a “coat polypeptide” as defined herein is a polypeptide fragment of the coat protein that possesses coat protein function and additionally encompasses the full length coat protein as well or single-chain variants thereof.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

The term “stringent hybridization conditions” are known to those skilled in the art and can be found in Current Protocols in Molecular Biology, John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2.×SSC, 0.1% SDS at 50° C., preferably at 55° C., and more preferably at 60° C. or 65° C.

### Production of Virus-Like Particles

A more detailed description of the production of viral-like particles is presented hereinafter (e.g. Example 1). The general principles applicable to the production of viral-like particles can be summarized as follows.

Phage display is one of several technologies that make possible the presentation of large libraries of random amino acid sequences with the purpose of selecting from them peptides with certain specific functions. The basic idea is to create recombinant bacteriophage genomes containing a library of randomized sequences genetically fused to one of the structural proteins of the virion.

When such recombinants are transfected into bacteria each produces virus particles that display a particular peptide on their surface and which package the same recombinant genome that encodes that peptide, thus establishing the linkage of genotype and phenotype essential to the method. Arbitrary functions (e.g. the binding of a receptor, immunogenicity) can be selected from such libraries by the use of biopanning and other techniques. Because of constraints imposed by the need to transform and subsequently cultivate bacteria, the practical upper limit on peptide library complexity in phage display is said to be around 10¹⁰-10¹¹ [Smothers et al., 2002, Science 298:621-622]. This requirement for passage through E. coli is the result of the relatively complex makeup of the virions of the phages used for phage display, and the consequent necessity that their components be synthesized and assembled in vivo. For example, display of certain peptides is restricted when filamentous phage is used, or not possible, since the fused peptide has to be secreted through the E. coli membranes as part of the phage assembly apparatus.

### Bacteriophages

Properties of single-strand RNA bacteriophages are disclosed, e.g. in The Bacteriophages, Calendar, R L, ed. Oxford University Press. 2005. The known viruses of this group attack bacteria as diverse as E. coli, Pseudomonas and Acinetobacter. Each possesses a highly similar genome organization, replication strategy, and virion structure. In particular, the bacteriophages contain a single-stranded (+)-sense RNA genome, contain maturase, coat and replicase genes, and have small (<300 angstrom) icosahedral capsids. These include but are not limited to MS2, Qb, R17, SP, PP7, GA, M11, MX1, f4, Cb5, Cb12r, Cb23r, 7s and f2 RNA bacteriophages.

For purposes of illustration, the genome of a particularly well-characterized member of the group, called MS2, comprises a single strand of (+)-sense RNA 3569 nucleotides long, encoding only four proteins, two of which are structural components of the virion. The viral particle is comprised of an icosahedral capsid made of 180 copies of coat protein and one molecule of maturase protein together with one molecule of the RNA genome. Coat protein is also a specific RNA binding protein. Assembly may possibly be initiated when coat protein associates with its specific recognition target an RNA hairpin near the 5′-end of the replicase cistron. The virus particle is then liberated into the medium when the cell bursts under the influence of the viral lysis protein. The formation of an infectious virus requires at least three components, namely coat protein, maturase and viral genome RNA, but experiments show that the information required for assembly of the icosahedral capsid shell is contained entirely within coat protein itself. For example, purified coat protein can form capsids in vitro in a process stimulated by the presence of RNA [Beckett et al., 1988, J. Mol Biol 204: 939-47]. Moreover, coat protein expressed in cells from a plasmid assembles into a virus-like particle in vivo [Peabody, D. S., 1990, J Biol Chem 265: 5684-5689].

### Coat Polypeptide

The coat polypeptide encoded by the coding region is typically at least 120, preferably, at least 125 amino acids in length, and no greater than 135 amino acids in length, preferably, no greater than 130 amino acids in length. It is expected that a coat polypeptide from essentially any single-stranded RNA bacteriophage can be used. Examples of coat polypeptides include but are not limited to the MS2 coat polypeptide, R17 coat polypeptide (see, for example, Genbank Accession No P03612), PRR1 coat polypeptide (see, for example, Genbank Accesssion No. ABH03627), fr phage coat polypeptide (see, for example, Genbank Accession No. NP_{—}039624), GA coat polypeptide (see, for example, Genbank Accession No. P07234), Qb coat polypeptide (see, for example, Genbank Accession No. P03615), SP coat polypeptide (see, for example, Genbank Accession No P09673), f4 coat polypeptide (see, for example, Genbank accession no. M37979.1 and PP7 coat polypeptide (see, for example, Genbank Accession No PO363 0).

Examples of PP7 coat polypeptides include but are not limited to the various chains of PP7 Coat Protein Dimer in Complex With Rna Hairpin (e.g. Genbank Accession Nos. 2QUXR; 2QUXO; 2QUX_L; 2QUX_I; 2QUX_F; and 2QUX_C). See also Example 1 herein and Peabody, et al., RNA recognition site of PP7 coat protein, Nucleic Acids Research, 2002, Vol. 30, No. 19 4138-4144.

The coat polypeptides useful in the present invention also include those having similarity with one or more of the coat polypeptide sequences disclosed above. The similarity is referred to as structural similarity. Structural similarity may be determined by aligning the residues of the two amino acid sequences (i.e., a candidate amino acid sequence and the amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from a single stranded RNA virus, or can be produced using recombinant techniques, or chemically or enzymatically synthesized. Preferably, two amino acid sequences are compared using the BESTFIT algorithm in the GCG package (version 10.2, Madison Wis.), or the Blastp program of the BLAST 2 search algorithm, as described by Tatusova, et al. (FEMS Microbial Lett 1999, 174:247-250), and available at http://www.ncbi.nlm.nih.gov/blast/b12seq/b12.html. Preferably, the default values for all BLAST 2 search parameters are used, including matrix=BLOSUM62; open gap penalty=11, extension gap penalty=1, gap xdropoff=50, expect=10, wordsize=3, and optionally, filter on. In the comparison of two amino acid sequences using the BLAST search algorithm, structural similarity is referred to as “identities.”

Preferably, a coat polypeptide also includes polypeptides with an amino acid sequence having at least 80% amino acid identity, at least 85% amino acid identity, at least 90% amino acid identity, or at least 95% amino acid identity to one or more of the amino acid sequences disclosed above. Preferably, a coat polypeptide is active. Whether a coat polypeptide is active can be determined by evaluating the ability of the polypeptide to form a capsid and package a single stranded RNA molecule. Such an evaluation can be done using an in vivo or in vitro system, and such methods are known in the art and routine. Alternatively, a polypeptide may be considered to be structurally similar if it has similar three dimensional structure as the recited coat polypeptide and/or functional activity.

Heterologous peptide sequences (in the present invention, at least one CRLF-2 peptide as otherwise disclosed herein) inserted into the coat polypeptide or polypeptide may be a random peptide sequence. In a particular embodiment, the random sequence has the sequence Xaaₙ wherein n is at least 4, at least 6, or at least 8 and no greater than 20, no greater than 18, or no greater than 16, and each Xaa is independently a random amino acid. Alternatively, the peptide fragment may possess a known functionality (e.g., antigenicity, immunogenicity). The heterologous sequence may be present at the amino-terminal end of a coat polypeptide, at the carboxy-terminal end of a coat polypeptide, or present elsewhere within the coat polypeptide, preferably in the AB loop. Preferably, the heterologous sequence is present at a location in the coat polypeptide such that the inserted sequence is expressed on the outer surface of the capsid. In a particular embodiment, the peptide sequence may be inserted into the AB loop regions the above-mentioned coat polypeptides. Examples of such locations include, for instance, insertion of the heterologous peptide sequence into a coat polypeptide immediately following amino acids 11-17, or amino acids 13-17 of the coat polypeptide. In a most particular embodiment, the heterologous peptide is inserted at a site corresponding to amino acids 11-17 or particularly 13-17 of MS-2.

Alternatively, the heterologous peptide may be inserted at the N-terminus or C-terminus of the coat polypeptide. Any one or more of the CRLF-2 peptides as described herein may be used as the heterologous peptides for insertion into the coat polypeptide which produces a VLP expressing the inserted CRLF-2 peptide on its surface.

The heterologous peptide may be selected from the group consisting of a peptide that targets CRLF-2 and/or CD 19, a receptor for CRLF-2 and/or CD 19, a ligand which binds to a CRLF-2 and/or CD 19 cell surface receptor, a peptide with affinity for either end of a filamentous phage particle specific to CRLF-2 and/or CD 19, a metal binding peptide that binds to CRLF-2 and/or CD 19 or a CRLF-2 and/or CD 19 peptide with affinity for the surface of MS2. In preferred aspects, the heterologous peptide consists essentially of or is specifically MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO: 12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8). More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4).

In order to determine a corresponding position in a structurally similar coat polypeptide, the amino acid sequence of this structurally similar coat polypeptide is aligned with the sequence of the named coat polypeptide as specified above.

In a particular embodiment, the coat polypeptide is a single-chain dimer containing an upstream and downstream subunit. Each subunit contains a functional coat polypeptide sequence. The heterologous peptide may be inserted ton the upstream and/or downstream subunit at the sites mentioned herein above, e.g., A-B loop region of downstream subunit. In a particular embodiment, the coat polypeptide is a single chain dimer of an MS2 or PP7 coat polypeptide.

### Preparation of Transcription Unit

The transcription unit of the present invention comprises an expression regulatory region, (e.g., a promoter), a sequence encoding a coat polypeptide and transcription terminator. The RNA polynucleotide may optionally include a coat recognition site (also referred to a “packaging signal”, “translational operator sequence”, “coat recognition site”). Alternatively, the transcription unit may be free of the translational operator sequence.

The promoter, coding region, transcription terminator, and, when present, the coat recognition site, are generally operably linked. “Operably linked” or “operably associated with” refer to a juxtaposition wherein the components so described are in a relationship permitting them to function in their intended manner. A regulatory sequence is “operably linked” to, or “operably associated with”, a coding region when it is joined in such a way that expression of the coding region is achieved under conditions compatible with the regulatory sequence. The coat recognition site, when present, may be at any location within the RNA polynucleotide provided it functions in the intended manner.

The invention is not limited by the use of any particular promoter, and a wide variety of promoters are known. The promoter used in the invention can be a constitutive or an inducible promoter. Preferred promoters are able to drive high levels of RNA encoded by me coding region encoding the coat polypeptide Examples of such promoters are known in the art and include, for instance, T7, T3, and SP6 promoters.

The nucleotide sequences of the coding regions encoding coat polypeptides described herein are readily determined. These classes of nucleotide sequences are large but finite, and the nucleotide sequence of each member of the class can be readily determined by one skilled in the art by reference to the standard genetic code.

Furthermore, the coding sequence of an RNA bacteriophage single chain coat polypeptide comprises a site for insertion of a heterologous peptide as well as a coding sequence for the heterologous peptide itself. In a particular embodiment, the site for insertion of the heterologous peptide is a restriction enzyme site.

In a particular embodiment, the coding region encodes a single-chain dimer of the coat polypeptide. In a most particular embodiment, the coding region encodes a modified single chain coat polypeptide dimer, where the modification comprises an insertion of a coding sequence at least four amino acids at the insertion site. The transcription unit may contain a bacterial promoter, such as a lac promoter or it may contain a bacteriophage promoter, such as a T7 promoter and optionally a T7 transcription terminator.

In addition to containing a promoter and a coding region encoding a fusion polypeptide, the RNA polynucleotide typically includes a transcription terminator, and optionally, a coat recognition site. A coat recognition site is a nucleotide sequence that forms a hairpin when present as RNA. This is also referred to in the art as a translational operator, a packaging signal, and an RNA binding site. Without intending to be limiting, this structure is believed to act as the binding site recognized by the translational repressor (e.g., the coat polypeptide), and initiate RNA packaging. The nucleotide sequences of coat recognition sites are known in the art. Other coat recognition sequences have been characterized in the single stranded RNA bacteriophages R17, GA, Qβ, SP, and PP7, and are readily available to the skilled person. Essentially any transcriptional terminator can be used in the RNA polynucleotide, provided it functions with the promoter. Transcriptional terminators are known to the skilled person, readily available, and routinely used.

### Synthesis

The VLPs of the present invention may be synthesized in vitro in a coupled cell-free transcription/translation system. Alternatively VLPs could be produced in vivo by introducing transcription units into bacteria, especially if transcription units contain a bacterial promoter.

### Assembly of VLPs Encapsidating Heterologous Substances

As noted above, the VLPs of the present invention may encapsidate one or more peptides that target CRLF-2 and/or CD19. These VLPs play be assembled by performing an in vitro VLP assembly reaction in the presence of the heterologous substance. Specifically, purified coat protein subunits are obtained from VLPs that have been disaggregated with a denaturant (usually acetic acid). The protein subunits are mixed with the heterologous substance. In a particular embodiment, the substance has some affinity for the interior of the VLP and is preferably negatively charged.

Another method involves attaching the heterologous substance to a synthetic RNA version of the translational operator. During an in vitro assembly reaction the RNA will tightly bind to its recognition site and be efficiently incorporated into the resulting VLP, carrying with it the foreign substance.

In another embodiment, the substance is passively diffused into the VLP through pores that naturally exist in the VLP surface. In a particular embodiment, the substance is small enough to pass through these pores (in MS2 they are about 10 angstroms diameter) and has a high affinity for the interior of the VLP.

### VLP Populations

In the term “VLP populations or libraries”, “population” and “libraries” are used interchangeably and are thus deemed to be synonymous. In one particular embodiment, the library may be a random library; in another embodiment, the library is an CRLF-2 and/or CD 19-targeting peptide fragment library or a library of CRLF-2 and/or CD 19-targeting peptide fragments derived from CRLF-2 and/or CD19 polypeptides.

### Random Libraries (Populations)

Oligonucleotides encoding peptides may be prepared. In one particular embodiment, the triplets encoding a particular amino acid have the composition NNS where N is A, G, C or T and S is G or T or alternatively NNY where N is A, G, C, or T and Y is C or T. In order to minimize the presence of stop codons, peptide libraries can be constructed using oligonucleotides synthesized from custom trinucleotide phosphoramidite mixtures (available from Glen Research, Inc.) designed to more accurately reflect natural amino acid compositions and completely lacking stop codons.

### Antigen Fragment Libraries

An alternative strategy takes advantage of the existence of a cloned gene or genome to create random fragment libraries. The idea is to randomly fragment the gene (e.g. with DNasel) to an appropriate average size (e.g. −30 bp), and to blunt-end ligate the fragments to an appropriate site in coat polypeptide. In a particular embodiment, a restriction site may be inserted into the AB-loop or N-terminus of the coat polypeptide). Only a minority of clones will carry productive inserts, because they shift reading frame, introduce a stop codon, or receive an insert in antisense orientation, Any expression vector may in one embodiment contain a marker to pre-select clones with intact coat coding sequences. For example, GalE-strains of E. coli are defective for galactose kinase and accumulate a toxic metabolite when b-galactosidase is expressed in the presence of the galactose analogue, phenyl-b,D-galactoside (PGaI). Subjecting a random antigen-fragment library to selection for translational repressor function in the GalE-strain CSH41 F-containing pRZ5, a plasmid that fuses the MS2 replicase cistron's translational operator to lacZ will eliminate most undesired insertions by enriching the library for those that at least maintain the coat reading-frame.

### Synthesis

In a particular embodiment, VLP populations may be synthesized in a coupled in vitro transcription/translation system using procedures known in the art (see, for example, U.S. Pat. No. 7,008,651 Kramer et al., 1999, Cell-free coupled transcription-translation systems from E. coli, In. Protein Expression. A Practical Approach, Higgins and Hames (eds.), Oxford University Press). In a particular embodiment, bacteriophage T7 (or a related) RNA polymerase is used to direct the high-level transcription of genes cloned under control of a T7 promoter in systems optimized to efficiently translate the large amounts of RNA thus produced [for examples, see Kim et al., 1996, Eur J Biochem 239: 88 1-886; Jewett et al., 2004, Biotech and Bioeng 86: 19-26].

It is possible in a mixture of templates, particularly in the population of the present invention, different individual coat polypeptides, distinguished by their fusion to different peptides, could presumably package each other's mRNAs, thus destroying the genotype/phenotype linkage needed for effective phage display. Moreover, because each capsid is assembled from multiple subunits, formation of hybrid capsids may occur. Thus, in one preferred embodiment, when preparing the populations or libraries of the present invention, one or more cycles of the transcription/translation reactions be performed in water/oil emulsions (Tawfik et al., 1998, Nat Biotechnol 16: 652-6). In this now well-established method, individual templates are segregated into the aqueous compartments of a water/oil emulsion. Under appropriate conditions huge numbers of aqueous microdroplets can be formed, each containing on average a single DNA template molecule and the machinery of transcription/translation. Because they are surrounded by oil, these compartments do not communicate with one another. The coat polypeptides synthesized in such droplets should associate specifically with the same mRNAs which encode them, and ought to assemble into capsids displaying only one peptide. After synthesis, the emulsion can be broken and the capsids recovered and subjected to selection. In one particular embodiment, all of the transcription/translation reactions are performed in the water/oil emulsion. In one particular embodiment, only droplets containing only one template per droplet (capsids displaying only one peptide) is isolated. In another embodiment, droplets containing mixed capsids may be isolated (plurality of templates per droplet) in one or more cycles of transcription/translation reactions and subsequently capsids displaying only one peptide (one template per droplet) are isolated.

Selection of CRLF-2 and/or CD19-Targeting Candidates

The VLP populations or libraries of the present invention may be used to select CRLF-2 and/or CD19-targeting candidates. The libraries may be random or antigenic libraries. Libraries of random or alternatively antigen-derived peptide sequences are displayed on the surface of VLPs, and specific target epitopes, or perhaps mimotopes are then isolated by affinity-selection using antibodies. Since the VLPs encapsidate their own mRNAs, sequences encoding them (and their guest peptides) can be recovered by reverse transcription and PCR. Individual affinity-selected VLPs are subsequently cloned, over-expressed and purified.

Techniques for affinity selection in phage display are well developed and are directly applicable to the VLP display system of the present invention. Briefly, an antibody (or antiserum) is allowed to form complexes with the peptides on VLPs in a random sequence or antigen fragment display library. Typically the antibodies will have been labeled with biotin so that the complexes can be captured by binding to a streptavidin-coated surface, magnetic beads, or other suitable immobilizing medium.

After washing, bound VLPs are eluted, and RNAs are extracted from the affinity-selected population and subjected to reverse transcription and PCR to recover the coat-encoding sequences, which are then recloned and subjected to further rounds of expression and affinity selection until the best-binding variants are obtained. A number of schemes for retrieval of RNA from VLPs are readily imagined. One attractive possibility is to simply capture biotin-mAb-VLP complexes in streptavidin coated PCR tubes, then thermally denature the VLPs and subject their RNA contents directly to RT-PCR. Many obvious alternatives exist and adjustments may be required depending on considerations such as the binding capacities of the various immobilizing media. Once the selected sequences are recovered by RT-PCR it is a simple matter to clone and reintroduce them into E coli, taking care at each stage to preserve the requisite library diversity, which, of course, diminishes with each round of selection. When selection is complete, each clone can be over-expressed to produce a CRLF-2 and/or CD19-targeting VLP.

VLPs according to the present invention, as described above, express at least one CRLF-2 peptide on the surface of the VLP and preferably, include cargo, for example at least one anticancer drug (especially, for example, a chemotherapeutic agent as otherwise described herein which and optionally additional cargo such as one or more of a fusogenic peptide that promotes endosomal escape of VLPs and encapsulated DNA, other cargo comprising at least one cargo component selected from the group consisting of double stranded linear DNA or a plasmid DNA, an imaging agent, small interfering RNA, small hairpin RNA, microRNA, or a mixture thereof, wherein one of said cargo components is optionally conjugated further with a nuclear localization sequence. Any one or more of these components may be incorporated into VLPs readily using methods well-known in the art, including modifying the pac site of the bacteriophage RNA using crosslinking agents and conjugating the various components onto the crosslinking agents within the dimer coat polypeptide without impacting the ability of the coat polypeptide to spontaneously reassemble into VLPs as described in U.S. patent application Ser. No. 12/960,168, filed Dec. 3, 2010, entitled “Virus-Like Particles as Targeted Multifunctional Nanocarriers for Delivery of Drugs, Therapeutics, Sensors and Contrast Agents to Arbitrary Cell Types”, which is incorporated by reference in its entirety herein.

Bacteriophage VLPs such as MS2 and/or Qβ bacteriophages, also self-assemble into complete capsides in the presence of nucleic acids and thus, can be used to specifically encapsidate therapeutic RNA (e.g., shRNA, siRNA, antisense oligonucleotides, other microRNAs, ribozymes, RNA decoys, aptamers) and other RNA-modified cargos, including one or more RNA-modified cytotoxic agents (e.g., chemotherapeutic drugs or toxins) or one or more RNA-modified imaging agents (e.g. quantum dots). Typically, the nucleic acid is conjugated to one or more cytotoxic agents or one or more imaging agents using an appropriate crosslinking molecule as described herein.

For example, a chemotherapeutic agent, such as doxorubicin, can be conjugated to the pac site of MS2 using a heterobifunctional crosslinker molecule (e.g., NHA ester-maleimide agent, among others) to link a primary amine moiety present in doxorubicin or other chemotherapeutic agent to a nucleic acid molecule including, for example, the pac site) that is modified with a 3′ or 5′ sulfhydryl group. In exemplary approaches, cargo components, including, for example, drugs, therapeutic RNA as otherwise described herein, quantum dots, gold nanoparticles, iron oxide nanoparticles, etc. and other cargo, etc. can be linked to the thiolated pac site and incorporated with the capsids of the VLPs. Approaches for incorporating the various components into VLPs (preferably by conjugation through a crosslinking agent at the pac site) pursuant to the present invention are well-known in the art.

The efficacy and rate of capside assembly are maximized in the presence of the MS2 translational operator, a 19-nucleotide RNA stem-loop (SEQ ID NO:32, SEQ ID NO:33), that via its interaction with coat protein, mediates exclusive encapsidation of the MS2 genome during bacteriophage replication. See, Wu, et al., Bioconjugate Chemistry, 6(5):587-595 (1995); Pickett & Peabody, Nucl Acids. Res., 21 (19):4621-4626 (1993) and Uhlenback, Nature Structure Biology, 5(3):174-174 (1998). The MS2 operator, or pac site, can promote efficient encapsidation of non-genomic materials, such as the polypeptide toxins, including the A-chain of ricin toxin, among others, within the interior volume of MS2 VLPs upon conjugation of the pac site to the cargo of interest. MS2 VLPs will also encapsidate RNA hairpins with sequences that differ from that of the native operator, as well as heterologous nucleic acids, including singe- and double-stranded RNA and DNA less than 3 bkp in length. Accordingly, the sequence of the pac site can be modified as long as the modification does not prevent the RNA molecule from inducing VLP self assembly. For example, the pac site can further comprise a spacer molecule, such as a polyU nucleotide (e.g. (U)₃₋₉)).

Using known methods, a polyethylene glycol moiety may be attached to the VLPs or protocells as otherwise described herein. PEGylation sometimes assists in minimizing proteolytic degradation, reducing the humoral immune response against the capside protein and reducing non-specific interactions with non-target cells and thus, can help to increase the circulation half-life and enhance the bioavailability of the encapsidated cargo without appreciably affecting the specific affinity of the nanoparticle for their target cells.

The term “reporter” is used to describe an imaging agent or moiety which is incorporated into the phospholipid bilayer or cargo of protocells according to an embodiment of the present invention and provides a signal which can be measured. The moiety may provide a fluorescent signal or may be a radioisotope which allows radiation detection, among others. Exemplary fluorescent labels for use in protocells (preferably via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

The term “histone-packaged supercoiled plasmid DNA” is used to describe a preferred component of protocells according to the present invention which utilize a preferred plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the invention, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include GNQSSNFGPMKGGNFGGRSSGPYGGGGQYFAKPRNQGGY SEQ I.D NO: 28, RRMKWKK (SEQ ID NO:29), PKKKRKV (SEQ ID NO: 30), and KR[PAATKKAGQA]KKKK (SEQ ID NO:31), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA- or RNA-binding domains in nucleic acid-binding proteins. Nucl. Acids Res., 23, 1647-16561995); Weis, K. Importins and exportins: how to get in and out of the nucleus [published erratum appears in Trends Biochem Sci 1998 July; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html#tab2.

“Tyrosine kinase inhibitors” include, but are not limited to imatinib, axitinib, bosutinib, cediranib, dasatinib, erlotinib, gefitinib, lapatinib, lestaurtinib, nilotinib, semaxanib, sunitinib, toceranib, vandetanib, vatalanib, sorafenib (Nexavar®), lapatinib, motesanib, vandetanib (Zactima®), MP-412, lestaurtinib, XL647, XL999, tandutinib, PKC412, AEE788, OSI-930, OSI-817, sunitinib maleate (Sutent®)) and N-(4-(4-aminothieno[2,3-d]pyrimidin-5-yl)phenyl)-N-(2-fluoro-5-(trifluor-omethyl)phenyl)urea, the preparation of which is described in United States Patent Application Document No. 2007/0155758.

The term “tyrosine kinase inhibitors” is intended to encompass the hydrates, solvates (such as alcoholates), polymorphs, N-oxides, and pharmaceutically acceptable acid or base addition salts of tyrosine kinase inhibiting compounds.

The term “effective” is used herein, unless otherwise indicated, to describe an amount of a compound or composition which, in context, is used to produce or affect an intended result, whether that result relates to treating a subject who suffers from cancer and symptoms and conditions associated with cancer. This term subsumes all other effective amount or effective concentration terms which are otherwise described in the present application.

The term “inhibitory effective concentration” or “inhibitory effective amount” describes concentrations or amounts of compounds that, when administered according to the present invention, substantially or significantly inhibit aspects or symptoms of cancer or conditions associated with cancer.

The term “preventing effective amount” describes concentrations or amounts of compounds which, when administered according to the present invention, are prophylactically effective in preventing or reducing the likelihood of the onset of cancer or a condition associated with cancer or in ameliorating the symptoms of such disorders or symptoms. The terms inhibitory effective amount or preventive effective amount also generally fall under the rubric “effective amount”.

In certain embodiments, acute lymophblastic leukemia (ALL), including B-precursor acute lymphoblastic leukemia (B-ALL) is predicted to be either responsive or non-responsive to tyrosine kinase inhibitor mono or co-therapy based on a determination of whether it is likely to result in one or more of the clinical outcomes outlined in the following excerpts from the National Cancer Institute Childhood Acute Lymphoblastic Leukemia Treatment (PDQ®) (http://www.cancer.gov/cancertopics/pdq/treatment/childALL/HealthProfessional/Page2#Section_{—}526). (These clinical assessments and prognosis indicia are purely exemplary and are not limiting. Other clinical analyses may be employed in the determination of whether ALL, including B-precursor acute lymphoblastic leukemia (B-ALL) will respond to tyrosine kinase inhibitor mono or co-therapy.)

The rapidity with which leukemia cells are eliminated following onset of treatment and the level of residual disease at the end of induction are associated with long-term outcome. Because treatment response is influenced by the drug sensitivity of leukemic cells and host pharmacodynamics and pharmacogenomics, early response has strong prognostic significance. Various ways of evaluating the leukemia cell response to treatment have been utilized, including the following:

1. MRD determination.

2. Day 7 and day 14 bone marrow responses.

3. Peripheral blood response to steroid prophase.

4. Peripheral blood response to multiagent induction therapy.

5. Induction failure.

### MRD Determination.

Morphologic assessment of residual leukemia in blood or bone marrow is often difficult and is relatively insensitive. Traditionally, a cutoff of 5% blasts in the bone marrow (detected by light microscopy) has been used to determine remission status. This corresponds to a level of 1 in 20 malignant cells. If one wishes to detect lower levels of leukemic cells in either blood or marrow, specialized techniques such as PCR assays, which determine unique Ig/T-cell receptor gene rearrangements, fusion transcripts produced by chromosome translocations, or flow cytometric assays, which detect leukemia-specific immunophenotypes, are required. With these techniques, detection of as few as 1 leukemia cell in 100,000 normal cells is possible, and MRD at the level of 1 in 10,000 cells can be detected routinely.

Multiple studies have demonstrated that end-induction MRD is an important, independent predictor of outcome in children and adolescents with B-lineage ALL. MRD response discriminates outcome in subsets of patients defined by age, leukocyte count, and cytogenetic abnormalities. Patients with higher levels of end-induction MRD have a poorer prognosis than those with lower or undetectable levels. End-induction MRD is used by almost all groups as a factor determining the intensity of postinduction treatment, with patients found to have higher levels allocated to more intensive therapies. MRD levels at earlier (e.g., day 8 and day 15 of induction) and later time points (e.g., week 12 of therapy) also predict outcome.

MRD measurements, in conjunction with other presenting features, have also been used to identify subsets of patients with an extremely low risk of relapse. The COG reported a very favorable prognosis (5-year EFS of 97%±1%) for patients with B-precursor phenotype, NCI standard risk age/leukocyte count, CNS 1 status, and favorable cytogenetic abnormalities (either high hyperdiploidy with favorable trisomies or the ETV6-RUNX1 fusion) who had less than 0.01% MRD levels at both day 8 (from peripheral blood) and end-induction (from bone marrow).

There are fewer studies documenting the prognostic significance of MRD in T-cell ALL. In the AIEOP-BFM ALL 2000 trial, MRD status at day 78 (week 12) was the most important predictor for relapse in patients with T-cell ALL. Patients with detectable MRD at end-induction who had negative MRD by day 78 did just as well as patients who achieved MRD-negativity at the earlier end-induction time point. Thus, unlike in B-cell precursor ALL, end-induction MRD levels were irrelevant in those patients whose MRD was negative at day 78. A high MRD level at day 78 was associated with a significantly higher risk of relapse.

There are few studies of MRD in the CSF. In one study, MRD was documented in about one-half of children at diagnosis. In this study, CSF MRD was not found to be prognostic when intensive chemotherapy was given.

Although MRD is the most important prognostic factor in determining outcome, there are no data to conclusively show that modifying therapy based on MRD determination significantly improves outcome in newly diagnosed ALL.

### Day 7 and Day 14 Bone Marrow Responses.

Patients who have a rapid reduction in leukemia cells to less than 5% in their bone marrow within 7 or 14 days following initiation of multiagent chemotherapy have a more favorable prognosis than do patients who have slower clearance of leukemia cells from the bone marrow.

### Peripheral Blood Response to Steroid Prophase.

Patients with a reduction in peripheral blast count to less than 1,000/4 after a 7-day induction prophase with prednisone and one dose of intrathecal methotrexate (a good prednisone response) have a more favorable prognosis than do patients whose peripheral blast counts remain above 1,000/μL (a poor prednisone response). Poor prednisone response is observed in fewer than 10% of patients. Treatment stratification for protocols of the Berlin-Frankfurt-Münster (BFM) clinical trials group is partially based on early response to the 7-day prednisone prophase (administered immediately prior to the initiation of multiagent remission induction).

Patients with no circulating blasts on day 7 have a better outcome than those patients whose circulating blast level is between 1 and 999/μL.

### Peripheral Blood Response to Multiagent Induction Therapy.

Patients with persistent circulating leukemia cells at 7 to 10 days after the initiation of multiagent chemotherapy are at increased risk of relapse compared with patients who have clearance of peripheral blasts within 1 week of therapy initiation. Rate of clearance of peripheral blasts has been found to be of prognostic significance in both T-cell and B-lineage ALL.

### Induction Failure.

The vast majority of children with ALL achieve complete morphologic remission by the end of the first month of treatment. The presence of greater than 5% lymphoblasts at the end of the induction phase is observed in up to 5% of children with ALL. Patients at highest risk of induction failure have one or more of the following features:

T-cell phenotype (especially without a mediastinal mass).

B-precursor ALL with very high presenting leukocyte counts.

11q23 rearrangement.

Older age.

Philadelphia chromosome.

In a large retrospective study, the OS of patients with induction failure was only 32%. However, there was significant clinical and biological heterogeneity. A relatively favorable outcome was observed in patients with B-precursor ALL between the ages of 1 and 5 years without adverse cytogenetics (MLL translocation or BCR-ABL). This group had a 10-year survival exceeding 50%, and SCT in first remission was not associated with a survival advantage compared with chemotherapy alone for this subset. Patients with the poorest outcomes (<20% 10-year survival) included those who were aged 14 to 18 years, or who had the Philadelphia chromosome or MLL rearrangement. B-cell ALL patients younger than 6 years and T-cell ALL patients (regardless of age) appeared to have better outcomes if treated with allogeneic SCT after achieving complete remission than those who received further treatment with chemotherapy alone.”

The term “patient” or “subject” is used throughout the specification within context to describe an animal, generally a mammal and preferably a human, to whom treatment, including prophylactic treatment, according to the present invention is provided. For treatment of symptoms which are specific for a specific animal such as a human patient, the term patient refers to that specific animal.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the term “neoplasia” is used to describe all cancerous disease states and embraces or encompasses the pathological process associated with malignant hematogenous, ascitic and solid tumors. Representative cancers include, for example, stomach, colon, rectal, liver, pancreatic, lung (especially non-small cell lunger cancer), breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, bone cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Cancer which may be treated preferentially using compositions and/or methods which employ targeting peptides as otherwise disclosed herein include those cancers which express CRLF-2 receptors in an upregulated manner, including overexpression or hyperexpression of CRLF-2. Although the principal focus of the present application is on acute lymophoblastic leukemia (ALL) and in particular, B-cell ALL (B-ALL), any cancer which expresses CRLF-2 in an upregulated manner (including overexpression/hyperexpression) may be treated pursuant to the present invention.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be a tyrosine kinase inhibitor that is different from a tyrosine kinase inhibitor which has been previously administered to a subject. In many instances, the co-administration of another anti-cancer compound results in a synergistic anti-cancer effect.

Exemplary anti-cancer compounds for co-administration according to the present invention include anti-metabolites agents which are broadly characterized as antimetabolites, inhibitors of topoisomerase I and II, alkylating agents and microtubule inhibitors (e.g., taxol), as well as, EGF kinase inhibitors (e.g., tarceva or erlotinib) or ABL kinase inhibitors (e.g. imatinib). Anti-cancer compounds for co-administration also include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

The term “coadminister” “co-administration” or “combination therapy” is used to describe a therapy in which at least two active compounds in effective amounts are used to treat cancer or another disease state or condition as otherwise described herein at the same time. Although the term co-administration preferably includes the administration of two active compounds to the patient at the same time, it is not necessary that the compounds be administered to the patient at the same time, although effective amounts of the individual compounds will be present in the patient at the same time.

Co-administration of two or more anticancer agents will often result in a synergistic enhancement of the anticancer activity of the other anticancer agent, an unexpected result. One or more of the present formulations may also be co-administered with another bioactive agent (e.g., antiviral agent, antihyperproliferative disease agent, agents which treat chronic inflammatory disease, among others as otherwise described herein).

In one embodiment, the present invention is directed to high surface area (i.e., greater than about 600 m²/g, preferably about 600 to about 1,000-1250 mg²/g), preferably monodisperse spherical silica or other biocompatible material nanoparticles having diameters falling within the range of about 0.05 to 50 μm, preferably about 1,000 nm or less, more preferably about 100 nm or less, 10-20 nm in diameter, a multimodal pore morphology comprising large (about 1-100 nm, preferably about 2-50 nm, more preferably about 10-35 nm, about 20-30 nm) surface-accessible pores interconnected by smaller internal pores (about 2-20 nm, preferably about 5-15 nm, more preferably about 6-12 nm) volume, each nanoparticle comprising a lipid bilayer (preferably a phospholipid bilayer) supported by said nanoparticles (the phospholipic bilayer and silica nanoparticles together are labeled “protocells”), to which is bound at least one antigen which binds to a CRLF-2 and/or CD19 targeting polypeptide or protein on a cell to which the protocells are to be targeted, wherein the protocells further comprise (are loaded) with a small molecule anticancer agent and/or a macromolecule selected from the group consisting of a short hairpin RNA (shRNA), small interfering RNA (siRNA) or a polypeptide toxin (e.g. ricin toxin A-chain or other toxic polypeptide).

Small molecule anticancer agents and macromolecules (shRNAs, siRNAs other micro RNAs and polypeptide/proteins toxins) as otherwise described herein may be loaded by adsorption and/or capillary filling of the pores of the particle core. While the nanoparticles according to the present invention are preferably comprised of silica, they may be comprised of other materials organic or inorganic including (in addition to the preferred silica), alumina, titania, zirconia, polymers (e.g., polystyrene, polycaprolactone, polylactic and/or polyglycolic acid, etc.) or combinations thereof. In addition, the porous particles according to the present invention may also include inorganic particles, hydrogel particles or other suitable particles which may be added to influence the loading of the particle and/or the release of actives from the particle upon delivery in a biological system. In preferred embodiments, the porous particle core includes mesoporous silica particles which provide biocompatibility and nanoporosity. Nanoparticles pursuant to the present invention are otherwise described in PCT/US2010/020096, published as WO 21010/078569 on Jul. 8, 2011, which is incorporated by reference in its entirety herein. Mesoporous silica particles for use in the present invention may be preferred.

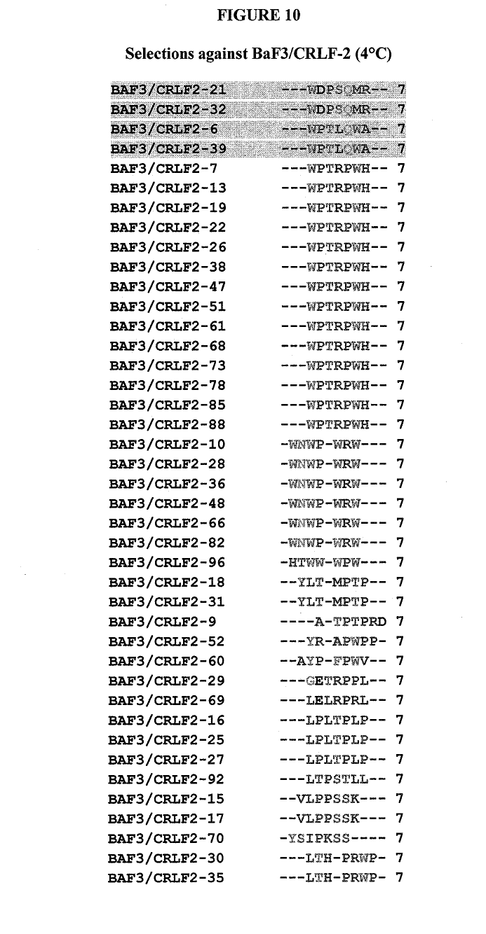

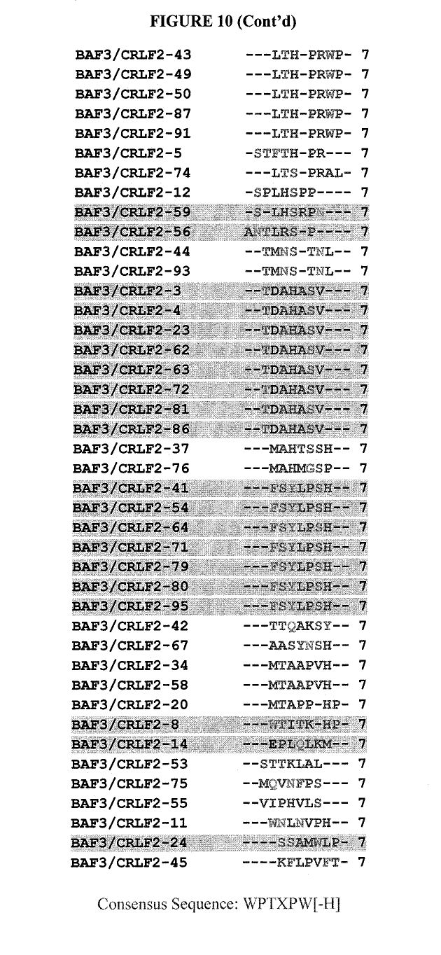

The term “CRLF-2 binding peptide” is used to describe any one or more of the peptides which are set forth in 3 or FIGS. 10-14 or equivalents thereof or as otherwise described herein. The term CD19 binding peptide is used to describe any one or more of the peptides which bind to CD19. The term CRLF-2 binding peptide is directed to peptides which consist essentially of/include the following specific peptides: MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13), HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) and NILSLSM (SEQ ID NO:22). Preferred CRLF-2 binding peptides include MTAAPVH (SEQ ID NO: 4), LTTPNWV (SEQ ID NO:5), AAQTSTP (SEQ ID NO:6), MHAPPFY (SEQ ID NO:10), ETKAWWL (SEQ ID NO:13), SQIFGNK (SEQ ID NO:15), AATLFPL (SEQ ID NO:11), TDAHASV (SEQ ID NO:7) and FSYLPSH (SEQ ID NO: 8). More preferably, the CRLF-2 binding peptide is MTAAPVH (SEQ ID NO: 4), LTTPNWV (SEQ ID NO:5), AAQTSTP (SEQ ID NO:6) or MHAPPFY (SEQ ID NO:10). Often, the CRLF-2 binding peptide used in embodiments according to the present invention includes MTAAPVH (SEQ ID NO: 4) and LTTPNWV (SEQ ID NO:5). Most often, the CRLF-2 binding peptide is MTAAPVH (SEQ ID NO: 4). Each of these polypeptides can be conjugated or otherwise covalently linked/complexed to protocells (for example, by modification of the peptide through insertion of a cysteinyl residue which can be reacted with a crosslinking agent as otherwise described herein or a hexameric histidine oligopeptide which can be complexed with an appropriately modified phospholipid which can complex copper and/or nickel to which the oligopeptide will bind).

As discussed above, each of these peptides may be conjugated/crosslinked to a protocell as otherwise described herein (preferably, to the phospholipid bilayer of the protocell) or inserted as a heterologous peptide into the peptide sequence of a bacteriophage coat polypeptide (which forms VLP's hereunder). In certain embodiments, as otherwise described herein, a hexameric histidine oligopeptide, a cysteinyl residue or is complexed/covalently linked through a spacer to the binding peptide. Generally, the spacer is between one and three amino acid residues (such as glycine, alanine that is non-functional but can provide spacing between the binding peptide and the group which assists in covalently linking/complexing the binding peptide to the protocell) in length inserted onto the carboxylic acid end of the peptide. The spacer allows the insertion of a functional group such as a cysteinyl residue or hexameric histidine oligopeptide which can assist in anchoring the binding peptide to the protocell.

Additional CRLF-2 binding sequences include consensus binding sequences which appear in an FIGS. 3 and 10-14 hereof, and include consensus peptide sequence WPTXPW[-H] (SEQ ID NO:25), ---S[FW][ST]XWXX--WX------ (SEQ ID NO:26), ------XSPXXWXXXXX-------- (SEQ ID No:27), FS--YLP[-S][-H] (SEQ ID NO: 34) and MT-AAP[VFW]H (SEQ ID NO:35),

It is noted that in certain instances the peptide contains unidentified amino acids, indicated as a dash (-) or an X in the peptide. In each instance, the unidentified amino acid may be substituted with any amino acid without affecting binding, preferably a small, neutral amino acid such as an alanine, glycine, etc., among others. A consequence sequence was generated for each group of binding peptides. A consensus sequence is a way of representing the results of a multiple sequence alignment, where related sequences are compared to each other, and similar functional sequence motifs are found. The consensus sequence shows which residues are conserved (are always the same), and which residues are variable.

In certain embodiments, the porous particle core may be hydrophilic and can be further treated to provide a more hydrophilic surface in order to influence pharmacological result in a particular treatment modality. For example, mesoporous silica particles according to the present invention can be further treated with, for example, ammonium hydroxide or other bases and hydrogen peroxide to provide significant hydrophilicity. The use of amine containing silanes such as 3-[2-(2-aminoethylamino)ethylamino]propyltrimethoxysilane (AEPTMS), among others, may be used to produce negatively charged cores which can markedly influence the cargo loading of the particles. Other agents may be used to produce positively charged cores to influence in the cargo in other instances, depending upon the physicochemical characteristics of the cargo.

In certain preferred embodiments, the lipid bilayer comprises a phospholipid selected from the group consisting of phosphatidyl choline, 1,2-Dioleoyl-3-Trimethylammonium-propane (DOTAP), 1,2-Dioleoyl-sn-Glycero-3-Phosphocholine (DOPC), 1,2-dioleoyl-sn-glycero-3-phosphoethanolamine-N-[methoxy(polyethylene glycol)-2000, 1,2-dioleoyl-sn-glycero-3-phosphoethanolamine (DOPE) or mixtures thereof. In addition to a phospholipid, including the specific phospholipids as otherwise described herein, the lipid bilayer may also comprise cholesterol (for structural integrity of the lipid bilayer) as well as polyethylene glycol lubricants/solvents (e.g. PEG 2000, etc.) to provide flexibility to the lipid bilayer. In addition to fusing a single phospholipid bilayer, multiple bilayers with opposite charges may be fused onto the porous particles in order to further influence cargo loading, sealing and release of the particle contents in a biological system.

In certain embodiments, the lipid bilayer can be prepared, for example, by extrusion of hydrated lipid films through a filter of varying pore size (e.g., 50, 100, 200 nm) to provide filtered lipid bilayer films, which can be fused with the porous particle cores, for example, by pipette mixing or other standard method.

In various embodiments, the protocell (nanoparticle to which a lipid bilayer covers or is otherwise fused to the particle) can be loaded with and seal macromolecules (shRNAs, siRNAs and polypeptide toxins) as otherwise described herein, thus creating a loaded protocell useful for cargo delivery across the cell membrane

In preferred aspects of the present invention, the protocells provide a targeted delivery through conjugation of certain CRLF-2 and/or CD19 targeting peptides onto the protocell surface, preferably by conjugation to the lipid bilayer surface. These peptides may be synthesized with C-terminal cysteine residues and conjugated to one or more of the phospholipids (especially, DOPE, which contains a phosphoethanolamine group) which comprise the lipid bilayer.

The invention is illustrated further in the following non-limiting examples.

### Example 1

### Protocells and VLPs as a Potential Treatment for ALL

Here we report the use of protocells and VLPs as a potential treatment for ALL, as well as other cancers. We have identified peptides that effectively bind CRLF-2, a receptor that has been found to be over-expressed in ALL, allowing for effective preferential targeting of the protocell to leukemic cells. This has been accomplished by affinity selection against either recombinant CRLF-2 or BaF3 cells that were transfected to over express CRLF-2. A variety of drugs has also been delivered to the cells, including (high potency, high toxicity) or AG490 (low toxicity, low potency). The in vitro results suggest that protocells are several order of magnitude more effective at combating this leukemia than free drugs alone. In-vivo experiments using SCID mice injected intravenously with BaF3/CRLF2 cells are currently underway.

### Protocell—Flexible Platform for Targeted Delivery

TEM image shows porous nanoparticle can serve as a support for lipid bilayers, which in turn seal contents within the core.

Protocells combine a high capacity for disparate cargos with a modifiable, biocompatible surface.

### Identification of Targeting Peptides

Phage display biopanning can be used to identify peptides with high specific binding to target cells and minimal non-specific binding.

After DNA sequencing is performed on 40 different clones identified via biopanning to selectively bind to target leukemia cells, Mimox software is used to align and determine consensus sequences. Clones from these groups are then used to evaluate binding constants.

Flow cytometry is used to evaluate binding of individual phage clones within consensus sequences. In this case, a saturable binding curve can be constructed, allowing for the determination of the dissociation constant (Kd) of phage displaying potentially specific peptides that bind cells with high levels of CRLF2 expression but not parental cell lines with minimal expression.

Following biopanning, sets of peptides can be grouped using alignment and determination of consensus sequences.

### Targeted Internalization of Protocells by Leukemia

1. Protocells bind to target cells with high specificity at low peptide densities due to fluid supported bilayer.
2. Become internalized via receptor mediated endocytosis utilizing internalization sequences.
3. Endosomal conditions facilitate dissociation of the supported lipid bilayer while endosomolytic
peptide promotes endosomal escape and subsequent cargo release into cytosol.
4. Cargo can be delivered to the nucleus by modification with a Nuclear Localization Signal (NLS).

Once a targeting peptide has been selected, human cells known to overexpress CRLF-2 (MHH CALL 4) are used to evaluate the binding constants of peptide that has been cross-linked to protocell-supported lipid bilayer (SLB). We find that we can obtain high specific binding (low Kd values) with low numbers of peptides when peptides are displayed on fluid lipids (DOIDA) within fluid bilayers (DOPC) as a result of the ability for peptides to be recruited to the binding site. When the targeting peptide is displayed on a non-fluid lipid (DSIDA) within a non-fluid bilayer (DSPC), an increased concentration of targeting peptide is required to obtain similar specific binding. Both of these combinations result in peptides being evenly distributed over the surface of the protocell SLB. DOIDA in DOPC DSIDA in DSPC SLB.

The same targeting peptide can also be displayed on protocell SLBs featuring mixtures of lipids which will form segregated domains which serve to increase the local concentration of peptide. When peptide is displayed on fluid lipids (DOIDA) within a non-fluid bilayer (DSPC), we see increased binding affinity (lower Kd) and lower peptide concentrations. This is due to the ability for peptides displayed on fluid lipid domains to be specifically be recruited to binding sites following concentration within the non-fluid bulk domain. Slightly decreased binding affinities are observed when peptides are displayed on non-fluid lipid domains (DSIDA) within fluid SLBs (DOPC), although overall affinities are quite high due to the increased local concentration of peptides resulting from domain formation. This further demonstrates the importance of local fluidity for optimal peptide-target interactions.

Targeted protocells can become internalized within target cells (MHH CALL 4, Mutz-5 and BaF3/CRLF-2) over time. However, overall internalization efficiency is only ˜20%, which could lead to non-specific cytotoxicity.

Targeted protocells featuring peptides displayed on a fluid lipid (DOIDA) domain within a non-fluid SLB (DSPC) show high binding affinity to target cells which over-express CRLF-2 (MHH Call 4, Mutz-5, and BaF3/CRLF-2). Minimal non-specific binding to control cells is seen. Non-specific binding of non-targeted protocells is also minimal.

Targeted protocells can become internalized within target cells (MHH CALL 4, Mutz-5 and BaF3/CRLF-2) over time. However, overall internalization efficiency is only ˜20%, which could lead to non-specific cytotoxicity.

Targeted protocells that are modified with R8 peptides, known to facilitate macropinocytosis, become internalized within target cells (MHH CALL 4, Mutz-5 and BaF3/CRLF-2) over time at over 90% efficiency.

Additionally, targeted protocells also displaying the R8 peptides show increased internalization kinetics, exhibiting internalization half-lives to targeted cells over 5× shorter than targeted protocells without R8 peptide. When coupled with the increased internalization efficiency conferred by the addition of the R8 peptide, non-specific cytotoxity should be minimized.

### Targeted Protocells Selectively Kill Cancer

CRLF2-specific protocells loaded with the chemotherapeutic agent, doxorubicin (DOX) induce apoptosis of CRLF2-positive cells (MHH CALL 4) but not CRLF-2 negative cells (MOLT 4); apoptotic cells are labeled with Alexa Fluor 647-labeled annexin V and the cell impermeant nuclear stain, Sytox Green in the conflocal fluorescence microscopy images. Protocells modified with the CRLF2-specific targeting peptide and the R8 peptide result in a low-level of non-specific cytotoxicity to CRLF2-negative cells (NALM-6, MOLT 4, Jurkat, and parental BaF3), however, as evidenced by LC90 values.

### Minimizing Non-Specific Cytotoxicity

The CRLF2-specific peptide, MTAAPVH, does not promote rapid internalization of protocells by CRLF2-positive cells. Therefore, we further modified protocells with the R8 peptide, which promotes non-specific macropinocytosis in a density-dependent fashion. The following strategies mitigate the undesired toxicity of DOX-loaded protocells modified with both the CRLF2-specific peptide and the R8 peptide to CRLF2-negative cells: (1) alter R8 density to decrease the amount of time required for CRLF2-positive cells to internalize surface-bound protocells and (2) attempt to select for CRLF2-specific peptides that promote internalization by CRLF2-positive cells upon protocell binding.

### Experimental Details

FIG. 1 illustrates how a protocell is a flexible platform for targeted delivery. The TEM image shows that a porous nanoparticle can serve as a support for lipid bilayers, which in turn seal contents within the core. The TEM image shows that a porous nanoparticle can serve as a support for lipid bilayers, which in turn seal contents within the core.

The original peptides used in these experiments were identified using the process of phage display. In this process, complex library of phage displaying random peptide sequences is allowed to bind to a cell-based selection target. Phage display biopanning was used to identify peptides with high specific binding to target cells and minimal non-specific binding. Unbound phage are washed away and bound phage are eluted and used to infect bacteria for amplification. This process can be carried out iteratively until a population of phages that tightly bind the target is obtained. Affinity selection via phage display is shown in FIG. 2. The left side of FIG. 2 is a schematic depicting affinity selection using a filamentous phage library (7-mer peptide sequences). Conditions used in affinity selection are described in the right side of FIG. 2.

A library of filamentous phage displaying a complex random library of peptides is created and allowed to bind to cells displaying the target surface marker. The sample is washed and the bound phage are eluted and subjected to negative selection against a parental cell line lacking the target surface marker. Bacterial cells are then infected with the reduced library for amplification and the process is repeated iteratively until an enriched population of binding phage is acquired.

The selections were carried out on BaF3 cells that had been induced to create the cell receptor CRLF2 and express it on their surface. Negative selections were carried out on the parental cell line (no CRLF2) in order to assure that the ligand identified was in fact binding to the desired target and not to other features on the surface of the cells.

Three peptide sequences were pulled from the enriched population of peptides that exhibited binding to CRLF2: TDAHASV, FSYLPSH, and MTAAPVH (best binder).

### Virus-Like Particles and Virus-Like Particles as a Display Platform

A recently developed alternative to phage display involves the use of Virus-Like Particles or VLPS. VLPs of the bacteriophage MS2 are constructed of 90 fused dimers That self-assemble into an icosahedral shell 23 nm in diameter. These perfectly monodisperse particles can be engineered to display a complex random library of peptides on their surfaces in either constrained (inserted into the A-B loop) or non-constrained (inserted at the N-terminus) conformation. Unlike the phage these particles are modeled after, VLPs are non-infectious. When they self-assemble, they encapsulate their own RNA. This not only allows for a mechanism of introducing cargo, but allows for particles with specific binding affinity to be isolated, the RNA extracted, reverse transcribed and amplified to allow for the production of more particles displaying that specific peptide (see FIG. 3). VLPs can be made using the techniques described in Hooker J M, Kovacs E W, Francis M B. Interior surface modification of bacteriophage MS2. J Am Chem Soc. 2004; 126:3718-9, or they can be made using techniques that are either well-known to those of ordinary skill in the art or as otherwise described herein.

More specifically, the MS2 viral shell is built from 90 coat protein dimers that, when expressed from a plasmid in E. coli, spontaneously self-assemble into an icosahedral shell of, e.g. 27.5 nm diameter. Two modes of display are possible. Foreign peptides are displayed by inserting them into coat protein's AB-loop. However, folding of the wild type coat protein does not generally tolerate such insertions and we found it necessary to engineer a more stable molecule. Taking advantage of the physical proximity in the dimer of the N-terminus of one subunit to the C terminus of the other, we duplicated the coat protein coding sequence and fused the two copies into a single reading frame, so that both halves of the dimer are produced as a single polypeptide. Covalently tethering the two halves monomers to one another greatly increased the protein's stability and dramatically improved its tolerance of foreign peptides inserted into the AB-loop of the downstream half of this “single-chain dimer”.

We find that in excess of 90% of clones with 6-mer, 8-mer, or 10-mer random peptide insertions yield properly assembled VLPs, each displaying 90 copies of a foreign peptide on its surface. Protein display is accomplished by genetically fusing a foreign sequence to one of coat protein's termini (usually the C-terminus). However, the presence of a fusion on every copy of coat protein may interfere with capsid assembly. Therefore, our strategy is to fuse the foreign protein to the C-terminus of coat protein with a stop codon between. A nonsense-suppressing tRNA causes occasional read through of the stop codon, and production of the fusion protein. Suppression is relatively inefficient, so that only a few percent of coat protein molecules actually contain the C terminal extension. Co-assembly of the wild-type and fusion proteins produces a VLP with an average of a few foreign molecules per particle. It is also possible to fuse the foreign protein directly to coat, without an intervening stop codon. In that case the fusion and non-fusion versions of the protein are separately produced and co-assembled into VLPs in vitro. In this proposal we describe display of single-chain antibodies (scFv's) for the B-cell specific surface antigens, CD19 and CD22, and of the CRLF2-specific ligand, thymic stromal lymphopoietin (TSLP) by this method.

It is important to note that each VLP encapsidates its own mRNA. This means that the nucleotide sequences encoding any particular VLP and its guest peptide or protein are contained within the particle itself, and can be recovered by reverse transcription and polymerase chain reaction, making possible the affinity selection scheme illustrated in FIG. 2. Random sequence peptide libraries, for example, can be subjected to bio-panning on any arbitrarily chosen target. Amplification and re-cloning of the selected sequences leads to the identification of peptide ligands specific for the target. To facilitate library construction and screening, we constructed a plasmid vector (pDSP62) that expresses coat protein at high levels from the bacteriophage T7 promoter. It confers resistance to kanamycin and normally replicates using a ColE1 origin. It also contains a M13 replication origin so that a single-stranded version of the plasmid can be produced after super-infection with an M13 helper phage. This allows the straight-forward production of complex random sequence peptide libraries by extension in vitro of mutagenic primers on circular single stranded templates using the efficient mutagenesis procedure of Kunkel et al., Rapid and efficient site-specific mutagenesis without phenotypic selection. Proc Natl Acad Sci USA 1985, 82:488-492.

To restrict the insertion of peptides to the AB-loop of the downstream half of the single-chain dimer, the upstream copy is a synthetic “codon-juggled” coat sequence containing the maximum possible number of silent mutations. Thus, mutagenic primers can be targeted to anneal specifically to the downstream site. Using this vector, random sequence timer, 7mer, 8mer and 10 mer libraries containing more than 1010 individual members have been produced. The high density of MS2 VLP display (90 peptides per particle) can make it difficult during affinity selection to discriminate peptides with high intrinsic binding affinities from those that have low affinity, but bind with high avidity by virtue of multiple weak interactions. To introduce valency control in the MS2 system we made an alternate version of the sc-dimer with a stop codon separating its two halves (in pDSP62(am)). This mutant normally produces only wild-type coat protein from its upstream half, but in the presence of a nonsense suppressor tRNA, a small percentage of ribosomes read through the stop codon to produce the entire sc-dimer with its guest peptide (remembering that the peptide is present only in the downstream half). Both the wild-type and sc-dimer proteins are synthesized from a single mRNA, which they encapsidate when they co-assemble into a mosaic VLP that displays about three peptides per particle on average. Using the MS2 VLP system three or four rounds of affinity selection against antibodies with known epitopes (e.g. the anti-Flag antibody, M2) yield peptides that closely mimic those epitopes.

### Cell-Based Targets for Affinity Selection of CRLF2-Binding Peptides.

Affinity selections on cellular targets are complicated by the heterogeneity of surface protein expression on mammalian cells, which frequently results in a large number of peptides that bind unsuitable targets. A major goal of the work we propose is to create an efficient selection/counter-selection scheme. To illustrate our strategy, the gene encoding human CRLF2 was introduced into BaF3 cells (a murine IL3-dependent pro-B cell line) and the protein's abundant surface expression was confirmed by FACS (as shown in Project 2). The resulting BaF3-CRLF2 cells, together with the BaF3 parental line (which specifically lacks CRLF2), served as a matched selection/counter-selection pair and provided a convenient means of discarding affinity selectants that bind the many non-CRLF2 receptors inevitably encountered on the BaF3 surface. Affinity selections conducted in our laboratories using commercial M13 display libraries have identified a peptide ligand to CRLF2 (TDAHASV), which is shown in FIG. 3 to mediate the binding of M13 phage selectants to BaF3-CRLF2 with an apparent Kd of about 3 nM, while not showing significant binding to the BaF3 parent. As shown in Project 2, this CRLF2 targeting peptide has already been conjugated to protocells and we have demonstrated selective binding and toxicity in CRLF2-expressing ALL cell lines.

Targeting VLPs to Specific Cell Types with scFv's.

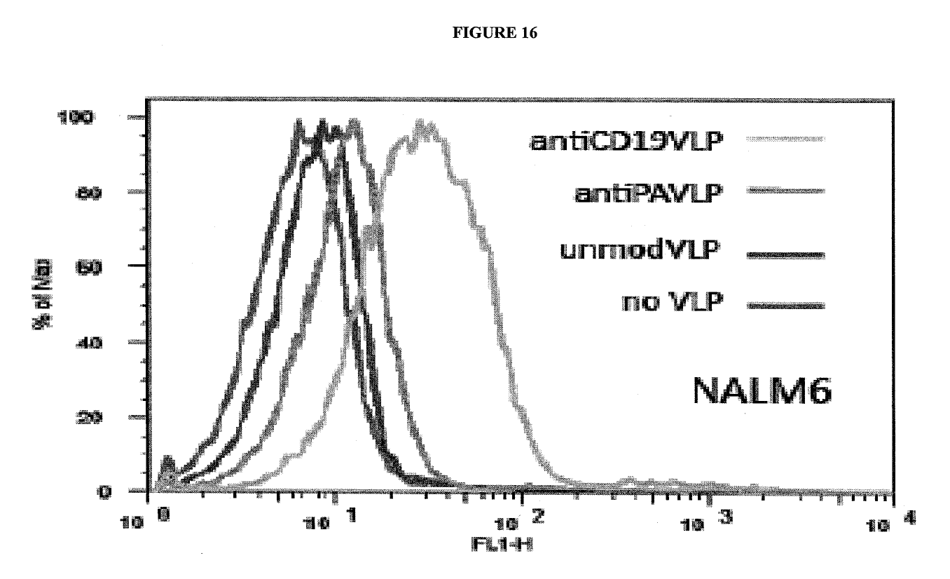

Monoclonal antibodies specific for a wide range of cell surface receptors represent a rich source of potential targeting molecules. We described above a system that enables the fusion a scFv to the C-terminus of coat protein, and which through nonsense suppression of a stop codon separating the two sequences, permits the simultaneous production from a single mRNA of coat protein and the scFv fusion. Co-assembly of the two proteins produces a VLP with an average of a few scFv molecules per particle. We have so far fused several different scFv's to coat protein and demonstrated the ability of the VLP-scFv to bind its target. Based on published amino acid sequences Cheng W W, Das D, Suresh M, Allen T M: Expression and purification of two anti-CD 19 single chain Fv fragments for targeting of liposomes to CD19-expressing cells. Biochim Biophys Acta 2007, 1768:21-29; Stemmer W P, Crameri A, Ha K D, Brennan T M, Heyneker H L: Single-step assembly of a gene and entire plasmid from large numbers of oligodeoxyribonucleotides. Gene 1995, 164:49-53, we synthesized (using assembly PCR Peabody, D. S. (2003) A viral platform for chemical modification and multivalent display. J. Nanobiotech. 1: 5.) an E. coli codon-optimized DNA sequence that encodes the anti-CD19 protein and fused it to the C-terminus of the MS2 coat protein sequence with an amber codon at the fusion junction. When this gene is expressed in bacteria with a suppressor tRNA, it produces large amounts of single-chain coat protein, and small amounts (a few percent) of the coat-scFv fusion, which co-assemble to yield a VLP displaying a few antibodies per particle, on average. Precise quantitation of the relative amounts of the two proteins has not yet been carried out, but this is one of the variables we seek to optimize with respect to particle yield, and cell binding and internalization. FACS analysis shows that the CD19-specific scFv directs VLPs to bind CD19+ cells (FIG. 16(b)). Future studies will characterize the affinity of the interaction and more carefully document its specificity.

FIG. 3 shows how flow cytometry is used to evaluate binding of individual phage clones within consensus sequences. In this case, a saturable binding curve can be constructed, allowing for the determination of the disassociation constant (K_{d}) of phage displaying potential specific peptides that bind cells with high levels of CRLF2 expression but not parental cell lines with minimal expression. Data shown for the MTAAPVH phage clone on BaF3/CRLF2 (A) and parental BaF3 cells. (B) Binding curves were constructed by titrating the amount of phage with constant cell concentrations in order to quantitatively describe binding. Non-specific binding was determined by incubating wild-type phage with the same cell lines. Specific binding to CRLF2 positive cells is significant; Specific binding to the parental cells is extremely minimal.

As shown in FIG. 3, following biopanning, sets of peptides can be grouped using alignment and determination of consensus sequences. After DNA sequencing is performed on 40 different clones identified via biopanning to selectively bind to target leukemia cells, Mimox software is used to align and determine consensus sequences (LEFT SIDE). Clones from these groups are then used to evaluate specific binding constants.

Flow cytometry is used to evaluate binding of individual phage clones within consensus sequences. In this case, a saturable binding curve can be constructed, allowing for determination of the dissociation constant (Kd) of phage displaying potentially specific peptides that bind cells with high levels of CRLF2 expression but not parental cell lines with minimal expression. (RIGHT SIDE)

VLPs are thought to be comparable to phage in their ability to conduct selections, and have successfully identified peptides that mapped directly onto the variable regions of specific antibodies. In addition, they are a suitable vehicle for delivery of cargo. Small molecule therapeutics and labels can be tagged with the RNA pac site that triggers self-assembly and thereby encapsulated within VLPs with relative ease.

CRLF2-targeting protocells were prepared using the techniques described herein and included the targeting and endosomolytic peptides and anticancer drugs (“cargo”) shown in FIG. 2. As depicted in FIGS. 1 and 2, porous nanoparticles can serve as a support for lipid bilayers, which in turn encapsulate CRLF2 and/or CD 19-targeting active ingredient(s) within the core.

The progression of this experiment aimed to identify binding peptides via phage display and to quantify their interactions with CRLF2 expressing cells while displayed on filamentous phage, genetically display these original peptides on the surface of VLPs, quantify the VLP interactions with CRLF2 expressing cells, and then identify peptides via the VLP based affinity selection process illustrated herein.

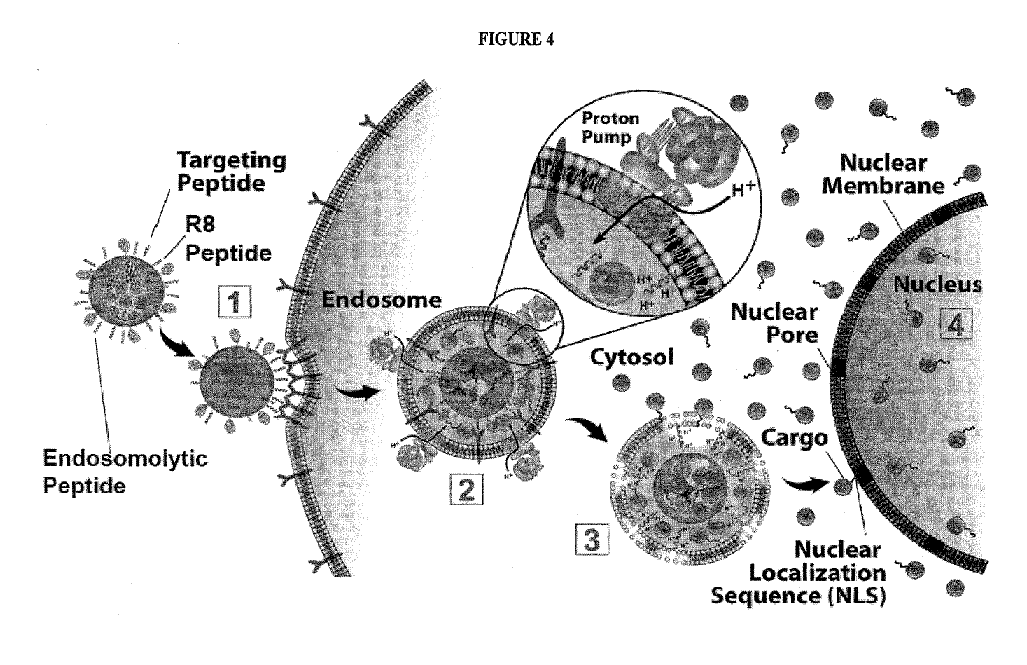

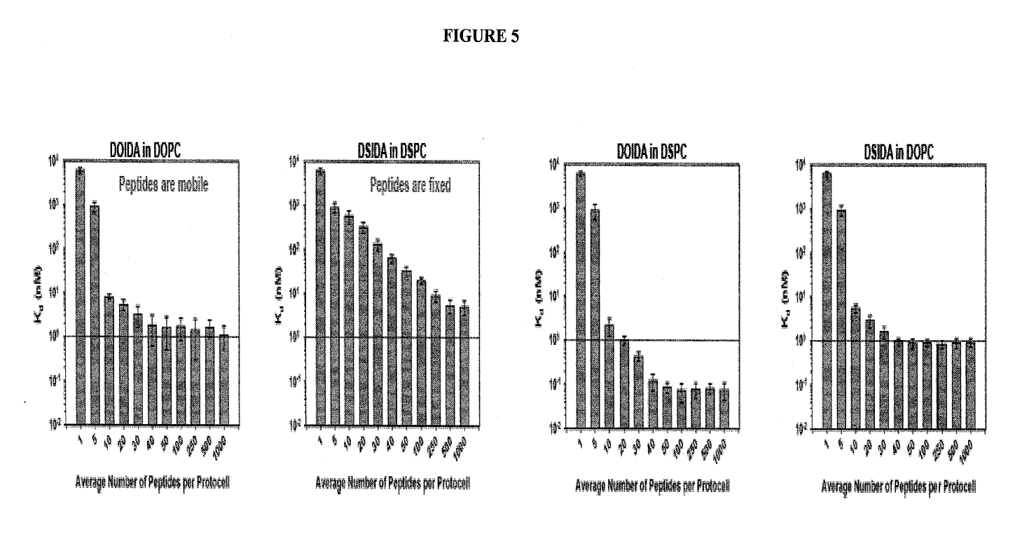

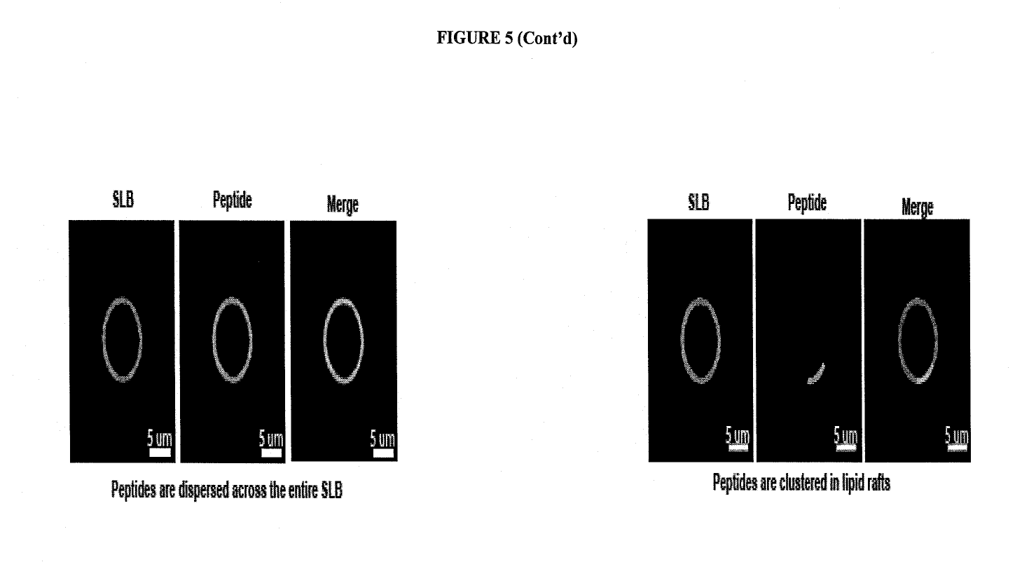

As shown in FIG. 4, it was determined that the protocells bind to target cells with high specificity at low peptide densities due to a fluid supported bilayer. FIG. 5 illustrates how once a targeting peptide has been selected, human cells known to over-express CRLF-2 (MMH CALL 4) were used to evaluate the binding constants of peptide that has been cross-linked to protocell-supported lipid bilayer (SLB). The same targeting peptide can also be displayed on protocell SLBs featuring mixtures of lipids which will form segregated domains which serve to increase the local concentration of peptide. When peptide is displayed on fluid peptides (DOIDA) within a non-fluid bilayer (DSPC), we see increased binding affinity (lower Kd) and lower peptide concentrations. This is due to the ability for peptides displayed on fluid lipid domains to be specifically be recruited to binding sites following concentration within the non-fluid bulk domain. Slightly decreased binding affinities are observed when peptides are displayed on non-fluid lipid domains (DSIDA) within fluid SLBs (DOPC), although overall affinities are quite high due to the increased local concentration of peptides resulting from domain formation. This further demonstrates the importance of local fluidity for optimal peptide-target interactions.

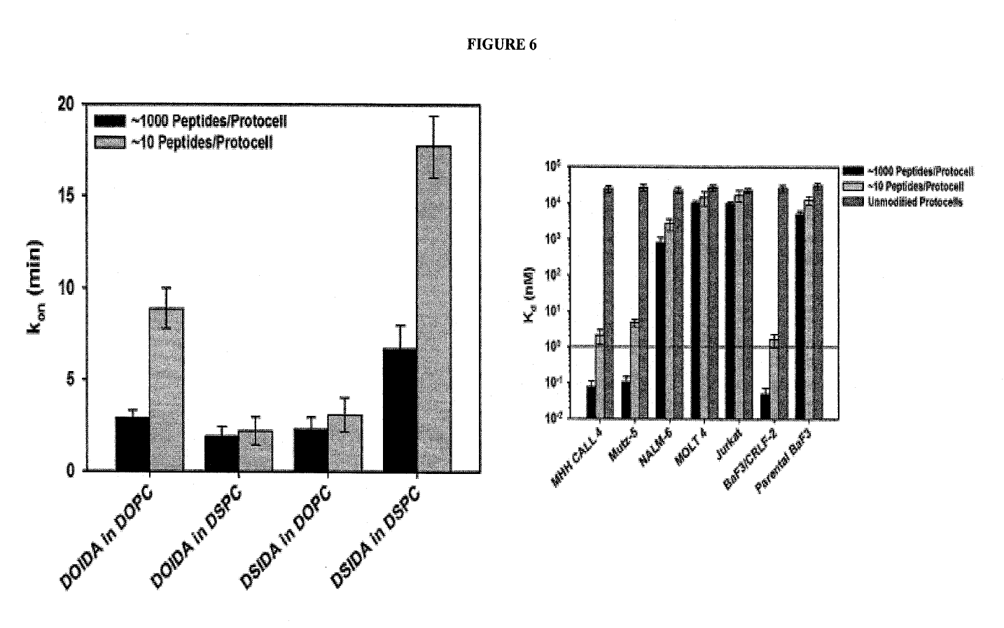

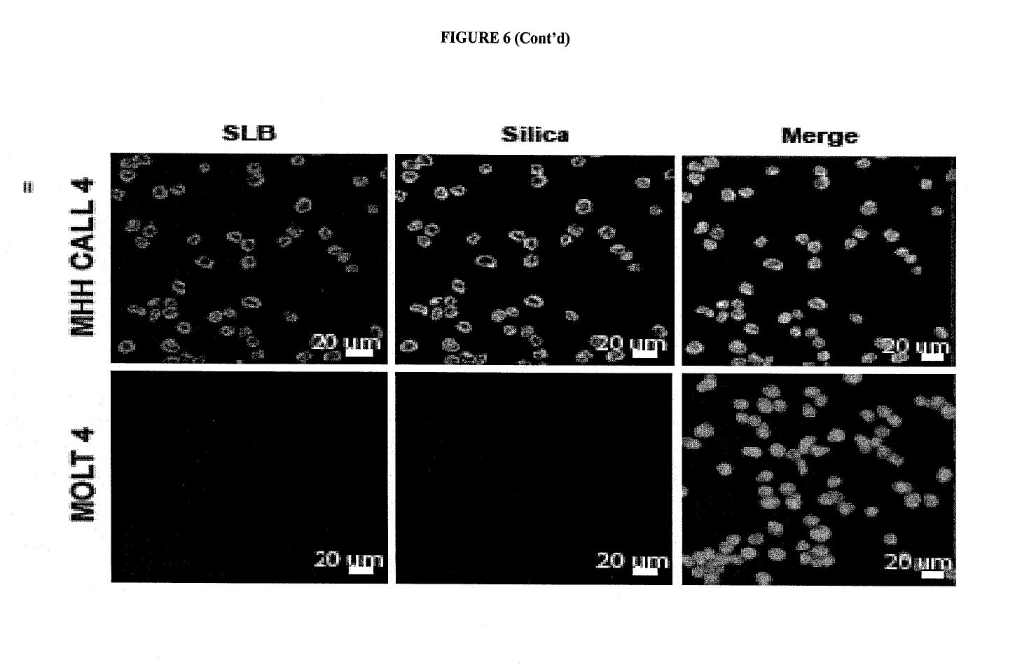

As illustrated in FIG. 6, targeted protocells can became internalized within target cells (MMH CALL 4, Mutz-5 and BaF3/CRLF-2) over time. The upper-right part of FIG. 6 depicts disassociation constants for CRLF-2 targeted protocells for various CRLF-2 positive and CRLF-2-negative cell lines. The lower portion of FIG. 6 shows images of CRLF-2-positive (MHH CALL 4) and CRLF-2-negative (MOLT 4) cells that were exposed to targeted protocells at 37° C. for two hours. Cells were pre-treated with cytochalasin D to inhibit internalization of surface-bound protocells. Bilayer composition=DOIDA in DSPC; all K_{d} and k(on) measurements were conducted at 37° C. using cells that had been exposed to cytochalasin D, which inhibits actin polymerization and, therefore, inhibits clathrin- and caveolae-dependent endocytosis, as well as macropinocytosis. Cells are labeled with CellTracker Green and DAPI in the microscopy images.

As shown in FIG. 6, targeted protocells featuring peptides displayed on a fluid lipid (DOIDA) domain within a non-fluid SLB (DSPC) show high binding affinity to target cells which over-express CRLF-2 (MHH Call 4, Mutz-5, and BaF3/CRLF-2). Minimal non-specific binding to control cells is seen. Non-specific binding of non-targeted protocells is also minimal.

The on-rates of different concentrations of targeting peptides displayed on fluid and non-fluid lipids within fluid and non-fluid lipid bilayers are also dependent on fluidity and local concentration. When peptides are displayed on lipids which form domains within the SLB, targeted protocells will more quickly reach a half-maximal level of saturated binding to target cells. This is a consequence of increased local concentration of targeting peptides on domains. Furthermore, if this domain remains fluid, half-maximal saturation is reached at an even faster rate.

FIG. 6 also shows that targeted peptides displayed on a fluid lipid (DOIDA) domain within a non-fluid SLB (DSPC) showed high binding affinity to target cells which over-express CRF-2 (MHH Cell 4, Mutz and BaF3/CRLF-2).

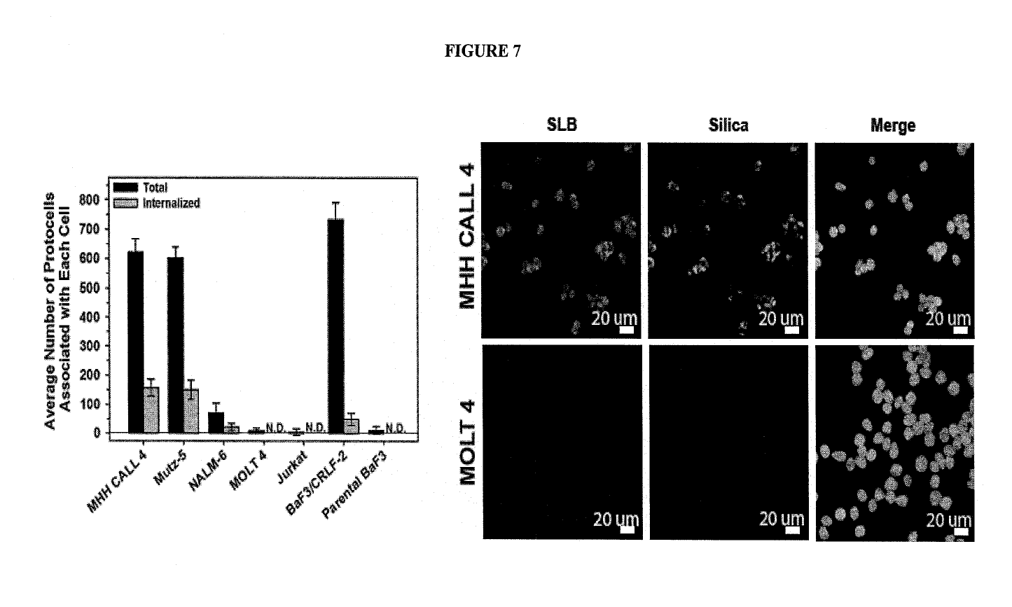

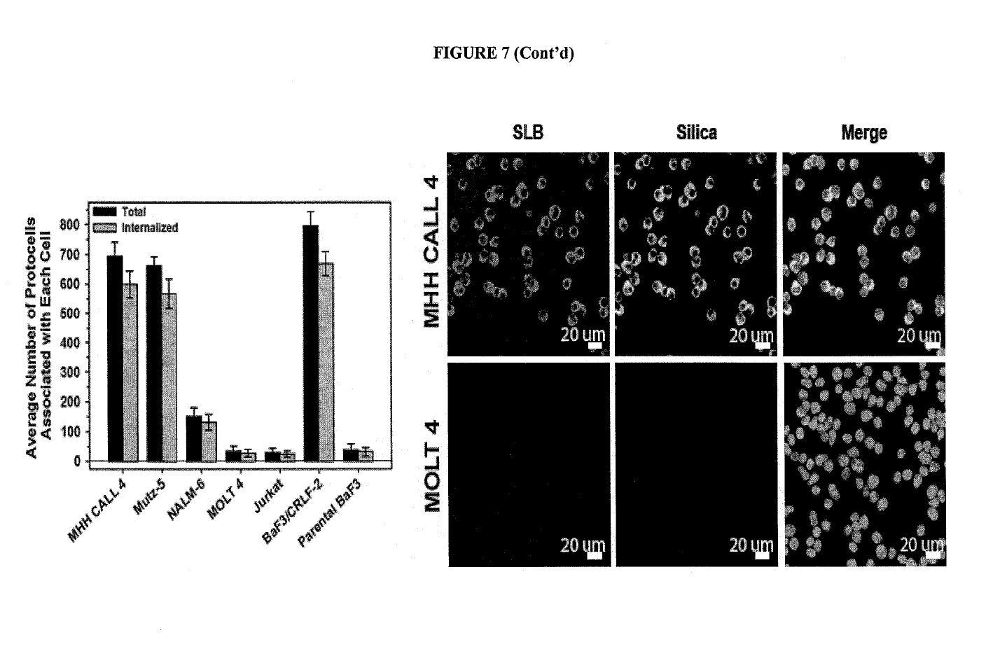

FIG. 7 also shows that targeted protocells became internalized within target cells (MHH CALL4, Mutz-5 and BaF3/CRLF-2) over time. More specifically, FIG. 7 illustrates that targeted protocells that are modified with R8 peptides, known to facilitate internalization, become internalized within target cells (MHH CALL 4, Mutz-5 and BaF3/CRLF-2) over time at over 90% efficiency.

The upper-left portion of FIG. 7 depicts the internalization efficacy of CRLF-2-targeted protocells in the absence of the R8 peptide. The upper right portion of FIG. 7 presents images of CRLF-2-positive (MHH CALL 4) and CRLF-2-negative (MOLT 4) cells exposed to targeted protocells for 37° C. for two hours. N.D.=not detectable; bilayer composition=DOIDA in DSPC with 5 wt % DSPE; approximate peptide density=10 targeting peptides/protocell and ˜500 H5WYG peptides/protocell. Targeted protocells can become internalized within target cells (MHH CALL 4, Mutz-5 and BaF3/CRLF-2) over time. However, overall internalization efficiency is only ˜20%, which could lead to non-specific cytotoxicity. The lower-left portion of FIG. 7 depicts the internalization efficacy of CRLF-2-targeted protocells in the presence of the R8 peptide. The lower right portion of FIG. 7 presents images of CRLF-2-positive (MHH CALL 4) and CRLF-2-negative (MOLT 4) cells exposed to targeted protocells for 37° C. for two hours. N.D.=not detectable; bilayer composition=DOIDA in DSPC with 5 wt % DSPE; approximate peptide density=10 targeting peptides/protocell and −500 H5WYG peptides/protocell.

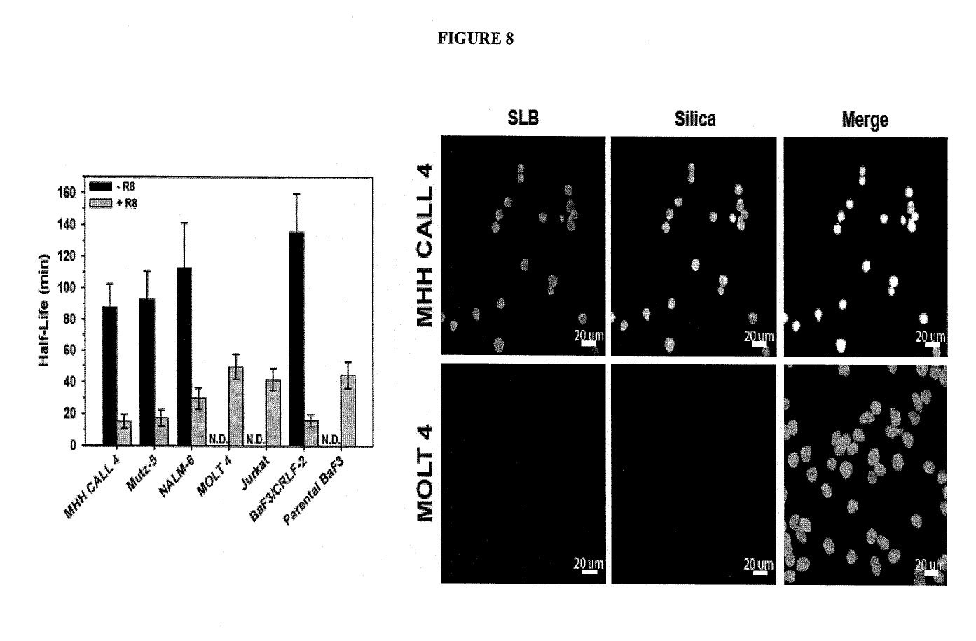

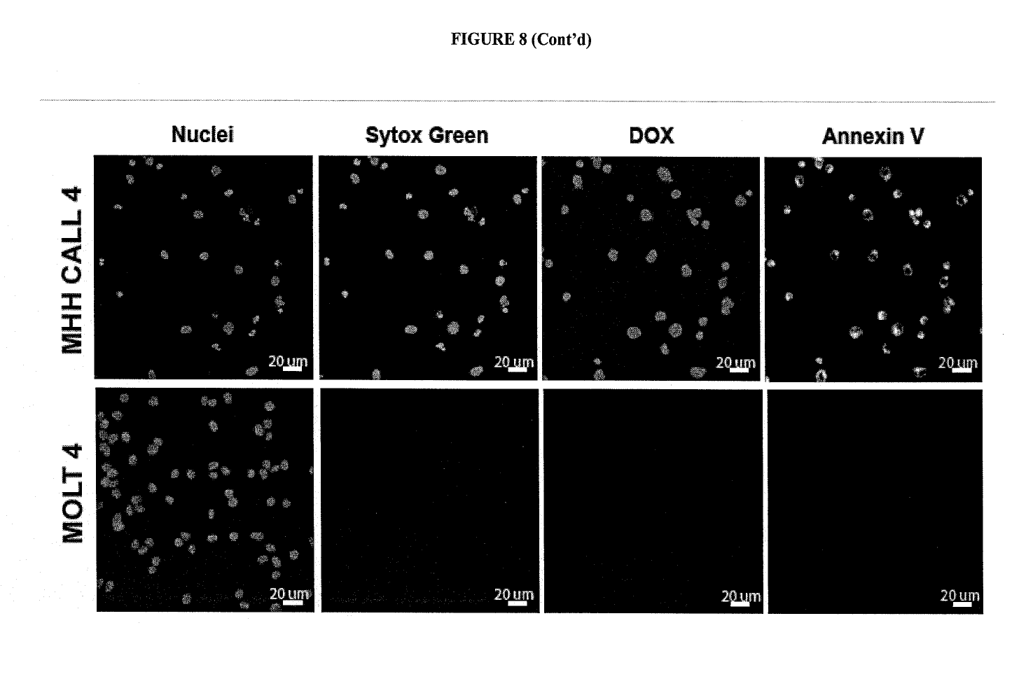

FIG. 8 illustrates that targeted protocells that display the R8 peptide showed increased internalization kinetics. The upper-left portion of FIG. 8 depicts internalization kinetics for CRLF-2-targeted protocells in the absence and presence of the R8 peptide. The upper-right portion of FIG. 8 depicts images of CRLF-2-positive (MHH CALL 4) cells exposed to targeted protocells at 37° C. for twenty-four hours. The bottom of FIG. 8 presents images of CRLF-2 positive (MHH CALL 4) and CRLF-2 negative (MOLT) that were continually exposed to 75 nM of DOX (encapsulated within CRLF-2-targeted, R8-modified protocells for 48 hours at 37° C. Bilayer composition=DOIDA in DSPC with 5 wt % DSPE; approximate peptide density=10 targeting peptides/protocell, ˜500 H5WYG peptides/protocell, and ˜500 R8 peptides/protocell.

Additionally, targeted protocells also displaying the R8 peptides show increased internalization kinetics, exhibiting internalization half-lives to targeted cells over 5× shorter than targeted protocells without R8 peptide. When coupled with the increased internalization efficiency conferred by the addition of the R8 peptide, non-specific cytotoxity should be minimized.

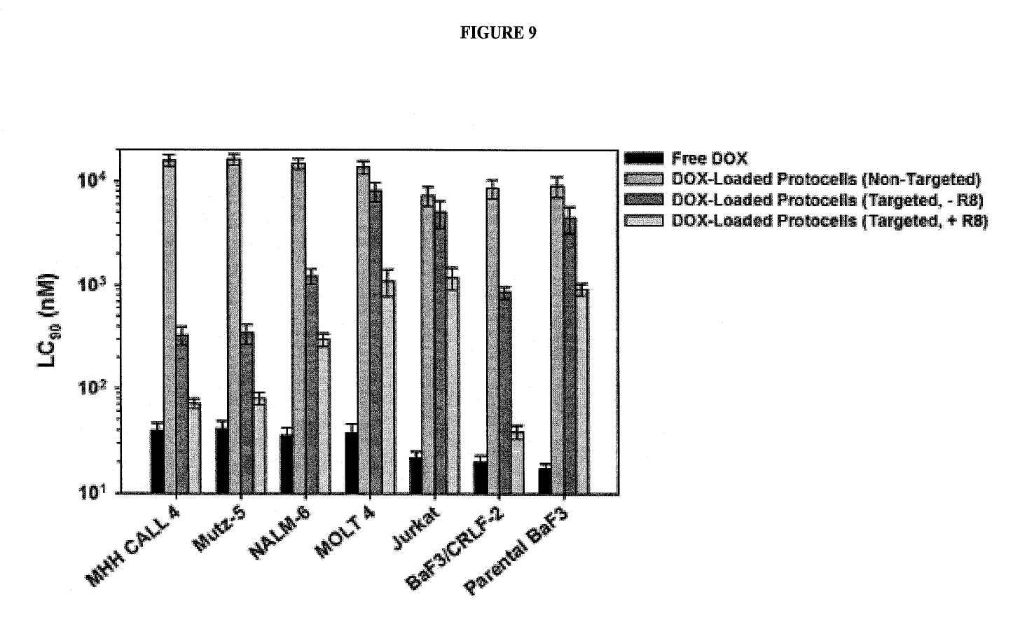

FIG. 9 illustrates that CRLF-2 specific protocells loaded with the chemotherapeutic agent doxorubicin (DOX) induced apoptosis of CRLF-2-positive cells (MHH CALL4) but not CRLF-2 negative cells (MOLT4).

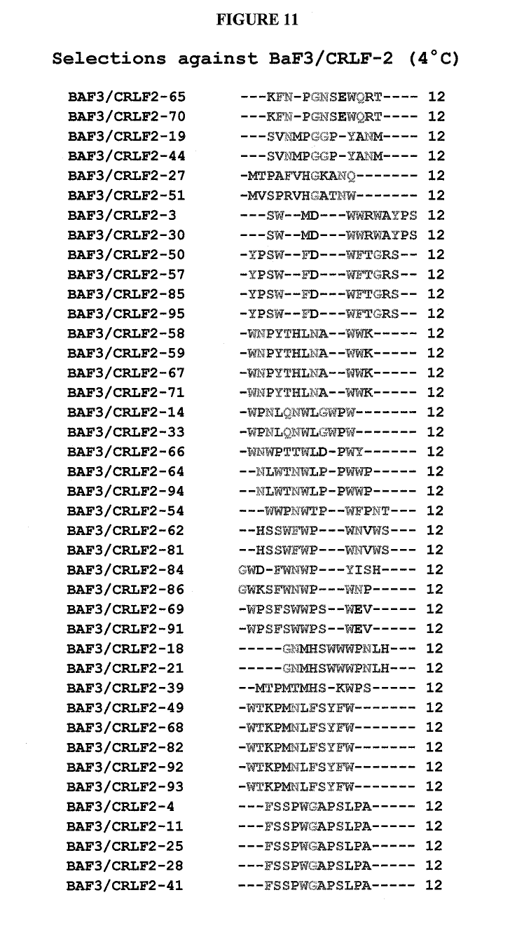

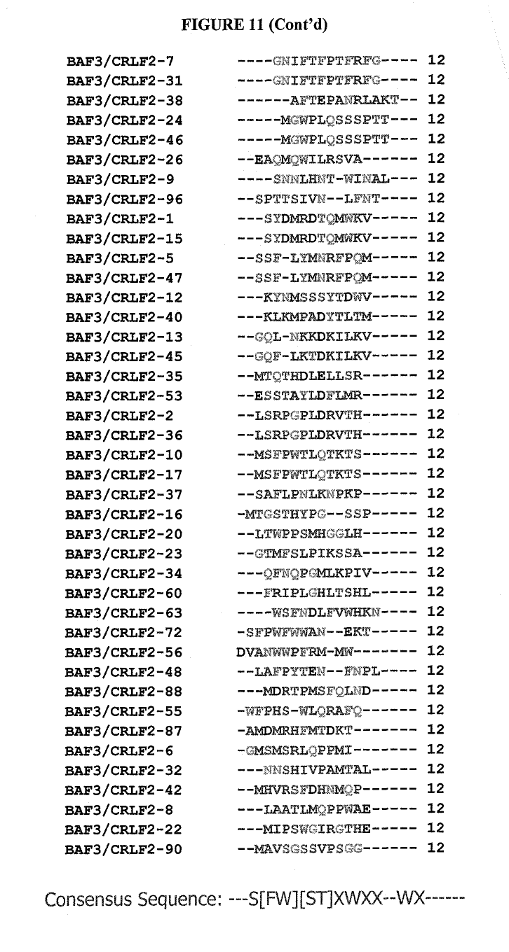

FIGS. 10 and 11 depict data for selections against BaF3/CRLF-2 (4° C.).

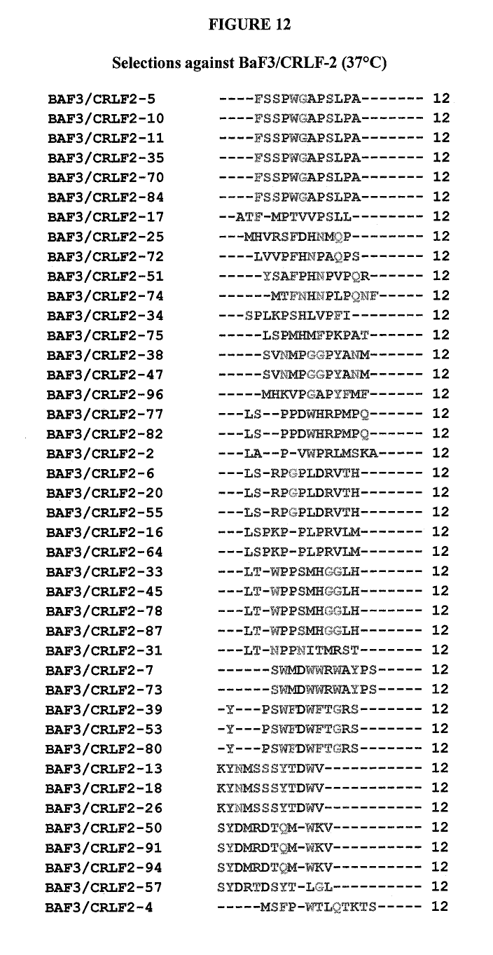

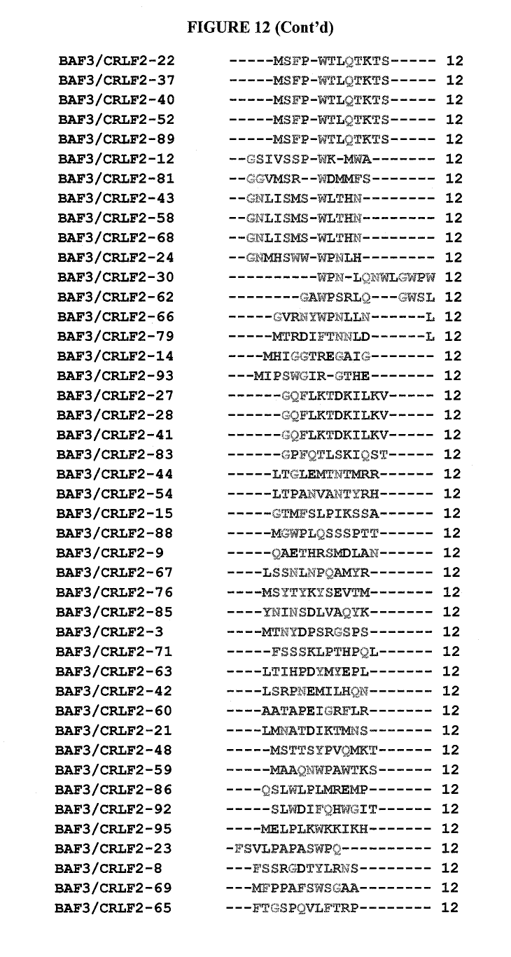

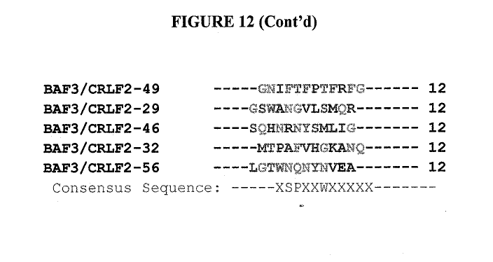

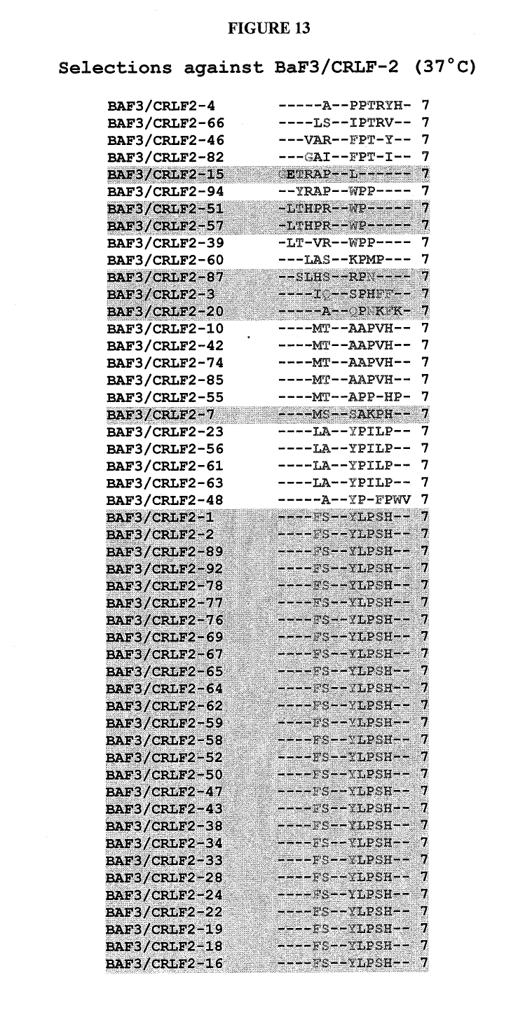

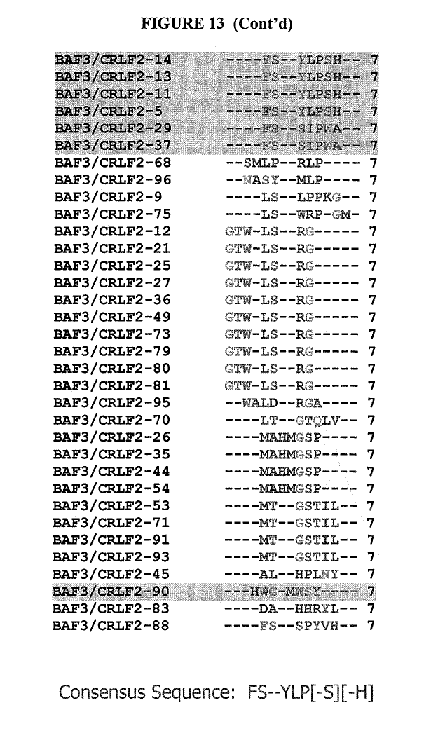

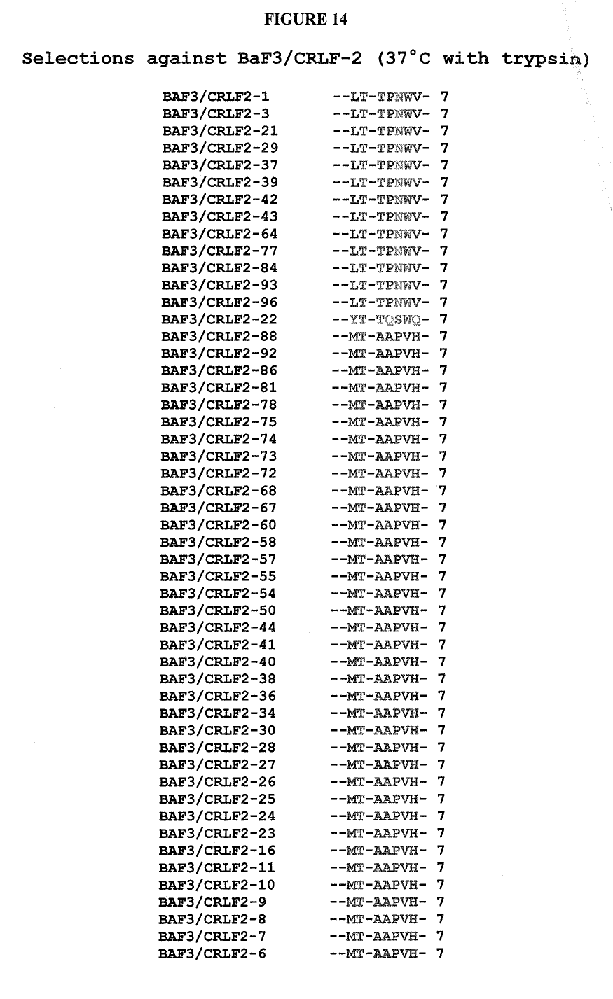

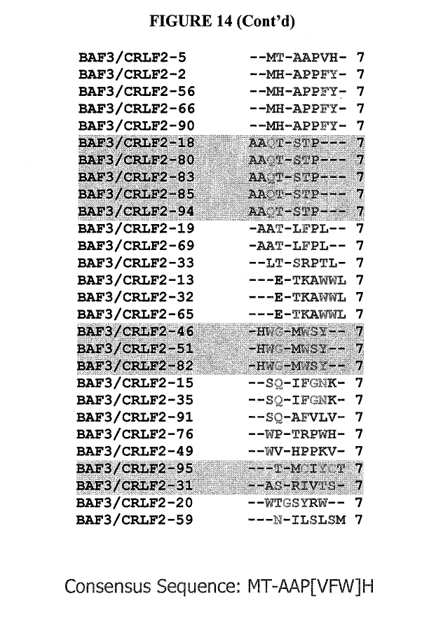

FIGS. 12 and 13 depict data for selections against BaF3/CRLF-2 (37° C.). FIG. 14 depicts data for selections against BaF3/CRLF-2 (37° C. with trypsin), as determined in the experiment(s) of Example(s).

### Quantification of Identified Peptides on MS2 VLPs

In order to quantify the original peptides on MS2 VLPs, the peptides first had to be genetically inserted in the coat protein dimer. This is done by designing primers that anneal to the DNA in a desired location and contain an insert coding for the peptide sequence to be displayed. Through PCR, restriction digests, and ligations, a new DNA strand, or plasmid, is produced. This plasmid can then be transformed into E. coli and induced to produce coat protein at a large scale. These proteins self-assemble into VLPs that can then be isolated and used to conduct experiments.

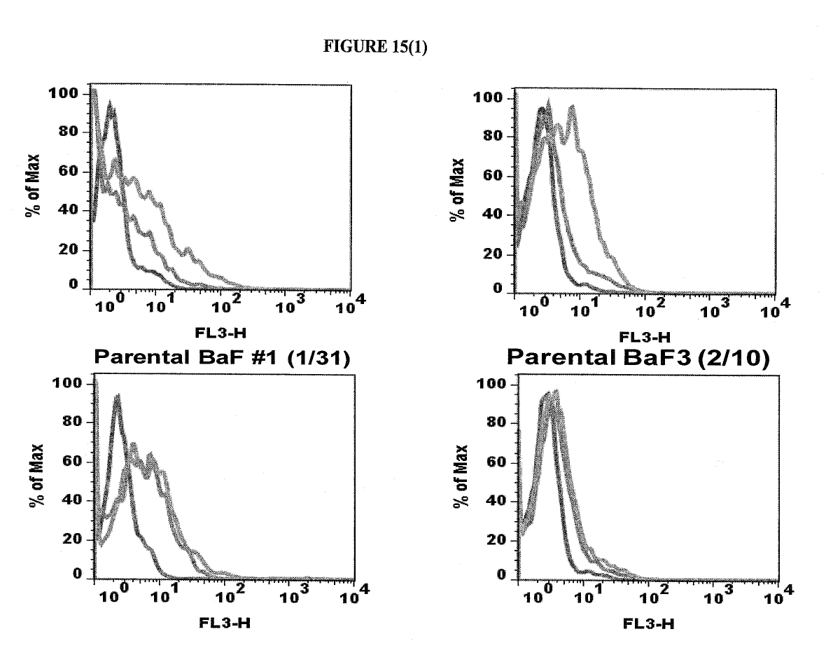

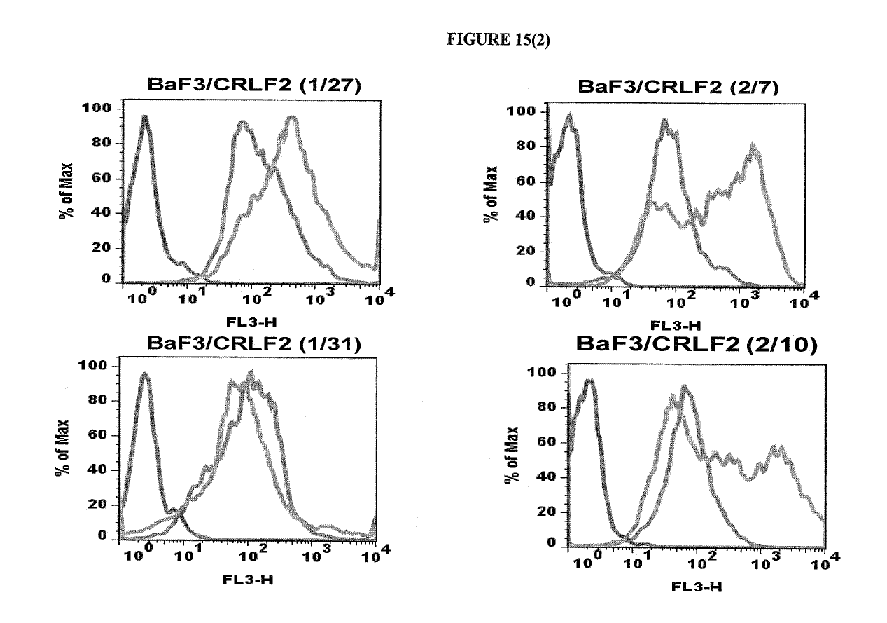

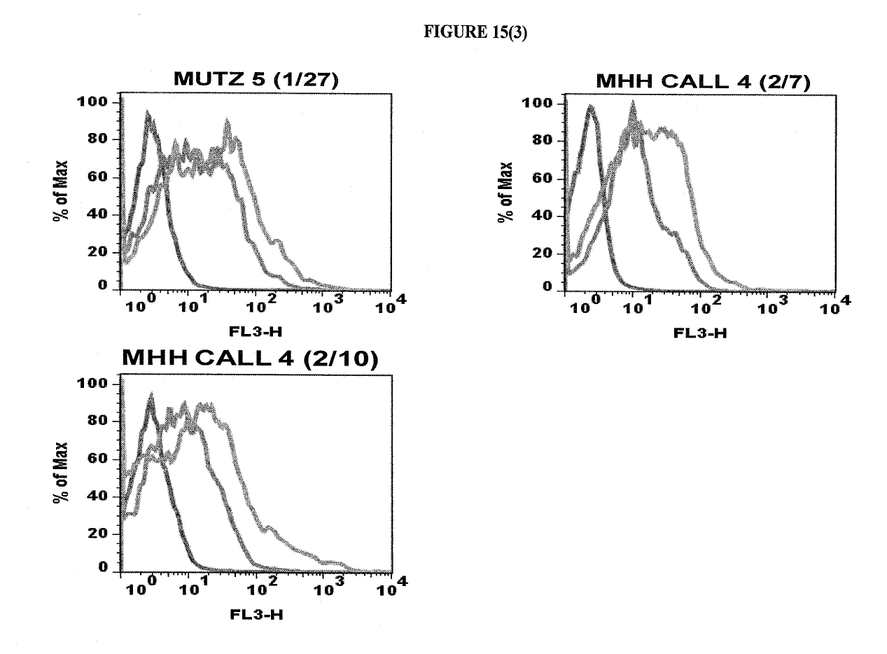

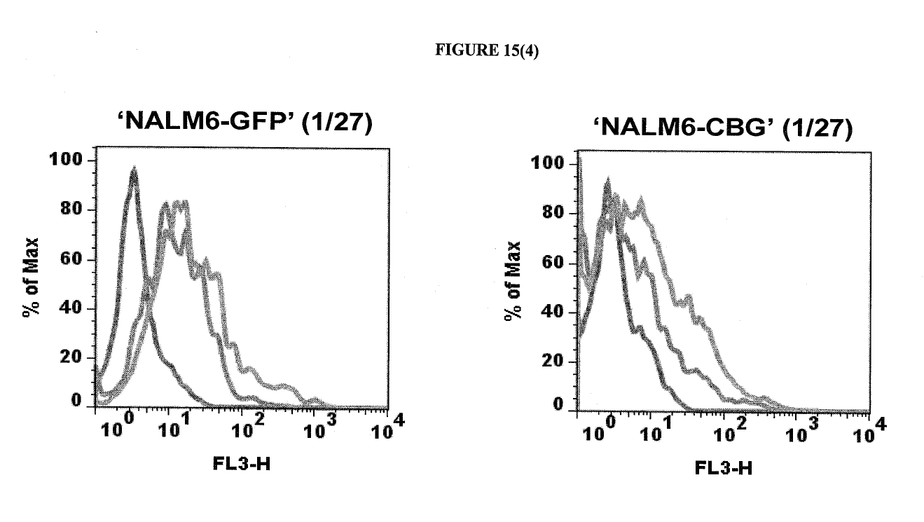

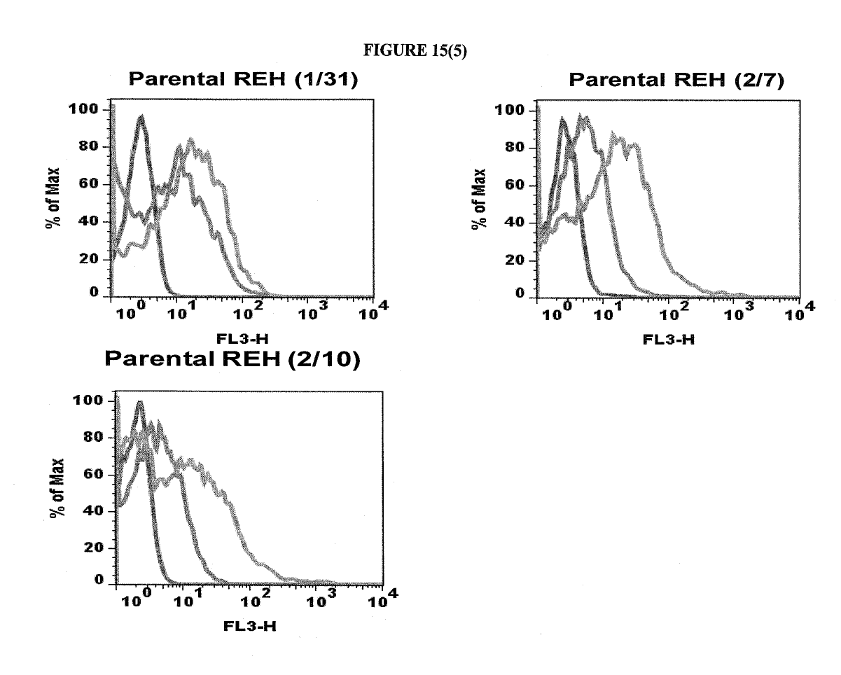

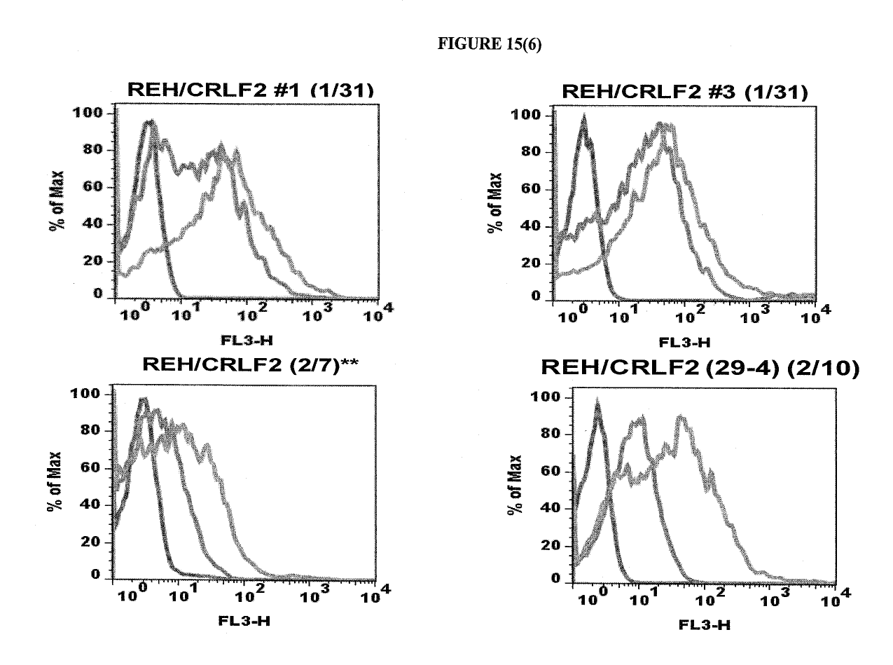

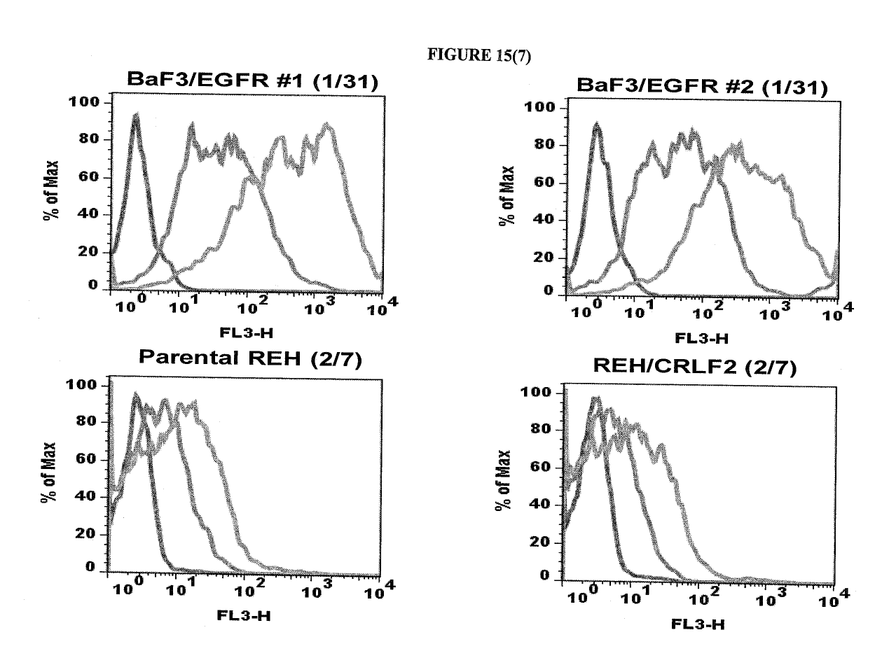

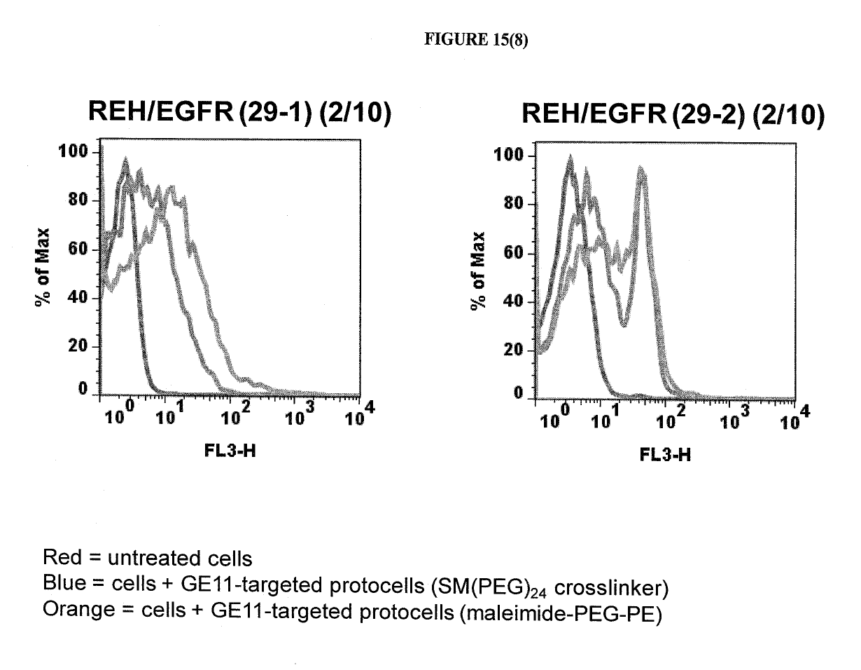

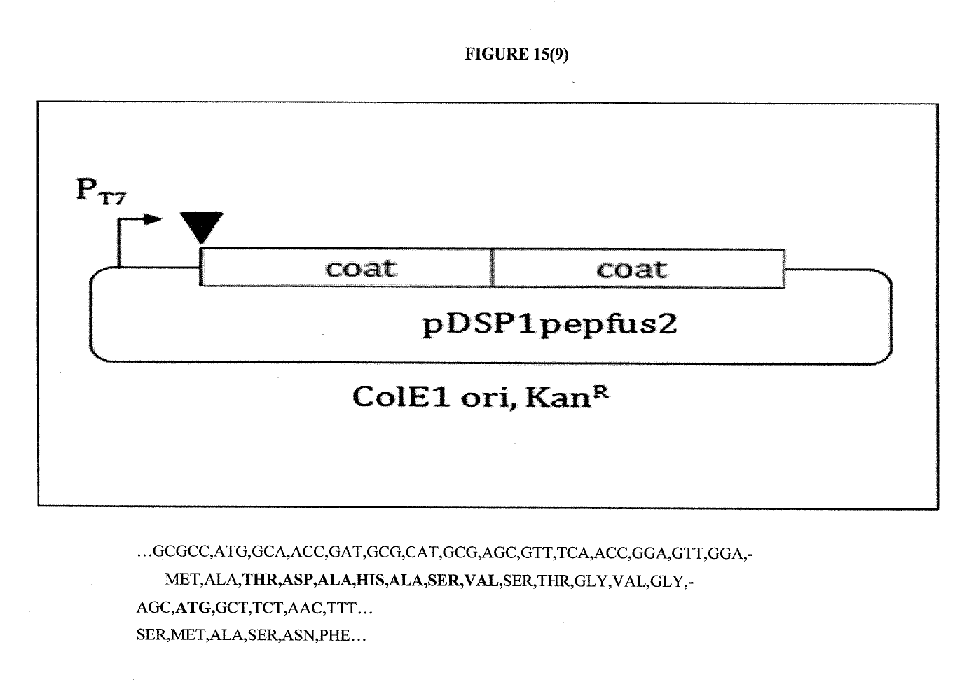

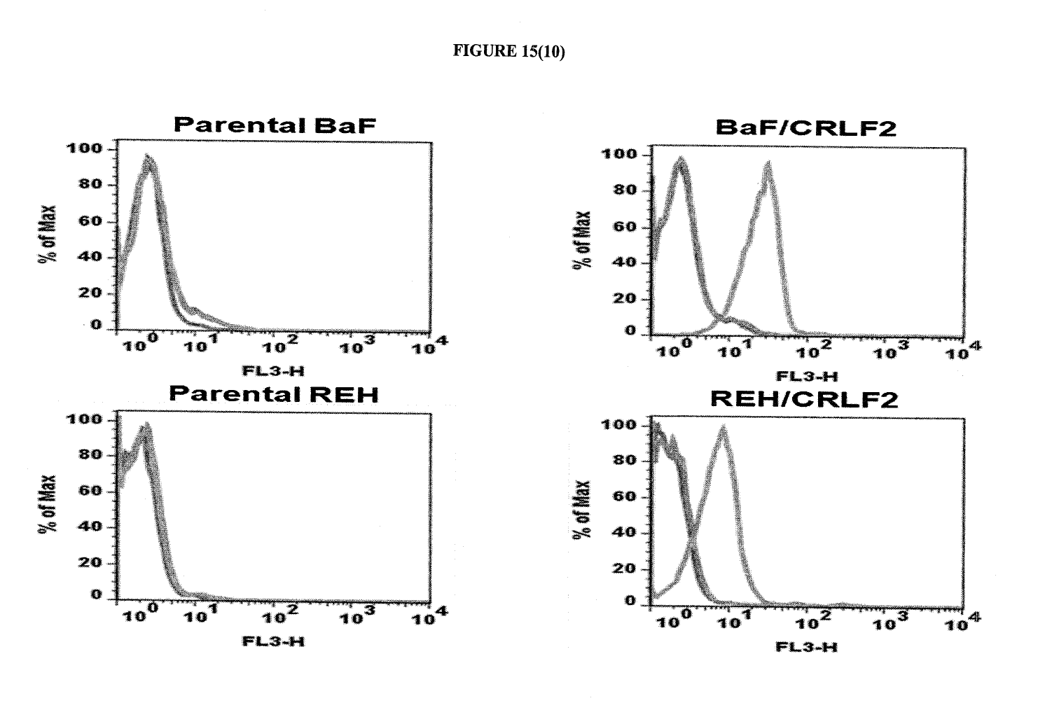

For flow cytometry experiments, particles were labeled with Alexa-fluor-647 and incubated with various cell types for an hour before the samples were washed and immediately measured using a FACSCaliber flow cytometer (data shown in FIGS. 15(1)-(8)). Samples included both targeted protocells (displaying the targeting peptides identified via phage display) and nontargeted protocells (displaying a non-relevant peptide and particles not displaying any additional peptides). These particles were screened against both BaF3/CRLF-2 and parental BaF3 cells. As expected, none of the samples demonstrated significant binding other than the targeted protocells incubated with target-expressing cells (lower right panel). To confirm binding, confocal microscopy images were taken of these samples as well.

Virus-like particle based affinity selection can be conducted using techniques similar to the phage display described above and depicted in FIG. 3, although VLPs are non-infectious. The RNA must be isolated from eluted VLPs, reverse transcribed into DNA, amplified, re-inserted into a plasmid encoding for coat protein which was then transformed into bacteria for production of particles.

### Validation of Display Platform by Targeting EGFR

Issues arose with trying to conduct VLP-based affinity selection on the cell expressing CRLF-2. Due to differences between the way that CRLF2 is displayed on the surface of naturally expressing cells and the way it is presented on the BaF3/CRLF2 cell line, as well as an incomplete knowledge of the receptor, it was difficult to determine if obstacles in the course of the research were due to flaws in the protocol itself, or in the presentation of the target. To this end, it was decided to proceed with VLP-based affinity selection on a better understood target: Epidermal Growth Factor Receptor (EGFR). Not only is EGFR well understood, it is clinically relevant. Anti-EGFR antibodies are currently being used to treat several varieties of cancer. Also, a new approach is required because treatments with these anti-bodies are beginning to lose effectiveness, and some studies suggest might activate the receptor leading to increased tumor motility. Selections are conducted against EGFR protein using a mixed library of VLPs displaying peptides of 6, 7, 8, and 10 amino acids in length. Prior to selection, the EGFR is affinity captured onto the surface of a microcell plate via a GST-tag. This increases not only the amount of protein adsorbed to the well, but also orients the proteins in such a manner as to increase the statistical likelihood of selecting for peptides that bind in the receptor binding pocket. Selections are currently in the middle of the third iteration of positive selection (selection against EGFR) and have undergone one round of negative selection (to reduce the number of VLPs in the propagated library that are binding to the glutathione on the surface of the wells). Comparison run during the negative selections confirm that the enriched library does include VLPs that selectively bind to EGFR.

A further experiment assessed the ability of a targeting peptide to direct the binding of a virus-like particle to CRLF2-producing cells, one was fused to the N-terminus of bacteriophage MS2 coat protein. The virus-like particle (VLP) thus produced was assayed for its ability to bind cells producing the targeted receptor. FIG. 15(9) shows the structure of a plasmid that expresses the MS2 coat protein single-chain dimer with a fusion of a CRLF2 targeting peptide (TDAHASV SEQ ID NO:7) at its N-terminus. This protein was produced from the plasmid in bacteria, where it spontaneously assembled into a VLP displaying 90 copies of the targeting peptide on its surface. To assess the particle's ability to bind cells with surface expressed CRLF2, two different cell lines (REH and BaF3) were stably transformed with the CRLF2 gene, thus producing REH-CRLF2 and BaF-CRLF2. Each of the parental cell lines expresses no CRLF2, but the derivatives express it abundantly. Purified VLPs were incubated with the various cell types, which were then treated with a fluorescently labeled antibody specific for MS2 coat protein. FACS analysis reveals the ability of the targeted VLPs to specifically bind only the cells producing CRLF2 (FIG. 15(10)).

### Example 2

### CD19 Protocol

CD19 IgG1 was partially reduced via reaction with a 60-fold molar excess of TCEP for 20 minutes at room temperature. Reduced antibody was then desalted and incubated with protocells (DOPC with 30 wt % cholesterol and 10 wt % maleimide-PEG-DMPE) overnight at 4 C. Protocells were washed 3× with PBS before being added to cells.

For flow cytometry experiments, particles were labeled with Alexa-fluor-647 and incubated with various cell types for an hour before the samples were washed and immediately measured using a FACSCaliber flow cytometer (data shown in FIG. 16). Samples included both targeted VLPs (displaying the targeting peptides identified via phage display) and nontargeted VLPs (displaying a non-relevant peptide and particles not displaying any additional peptides). These particles were screened against both BaF3/CRLF-2 and parental BaF3 cells. As expected, none of the samples demonstrated significant binding other than the targeted VLPs incubated with target-expressing cells (lower right panel).

### Example 3

### N2.v.1 ALL—a Model System to Understand and Perfect Targeted Delivery

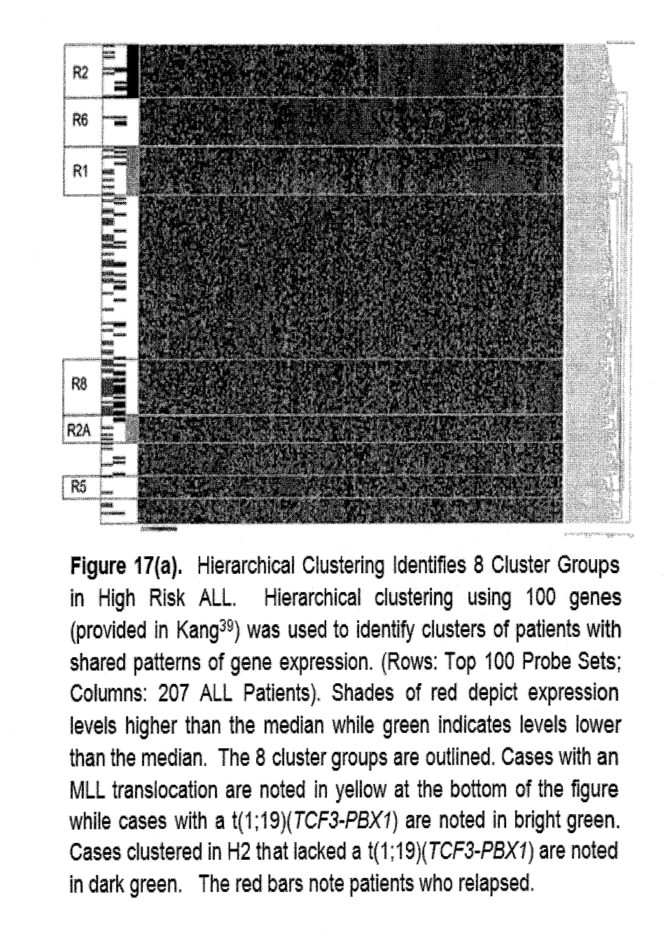

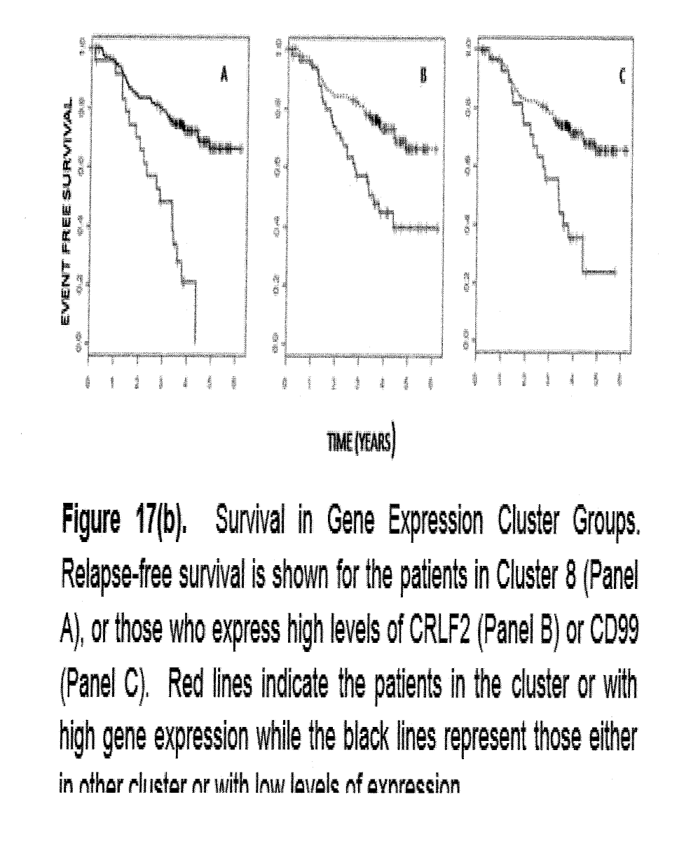

Diagnostic leukemic blast samples were obtained from 207 ALL patients enrolled in Children's Oncology Group (COG) trial 9906. These children had characteristics (older age and higher white blood count) that suggested that they were at an elevated risk of relapse (44% event free survival in earlier trials). RNA was extracted. Biotinylated cRNA was synthesized and hybridized to HG_U133A_Plus2.0 oligonucleotide microarrays, and fluorescent intensity signals were obtained for 54,688 probes sets corresponding to named genes and uncharacterized transcript). Final intensities were obtained after a standard masking and normalization procedure. “Outlier” genes, defined as transcripts expressed several logs above or below the mean in a subset of samples were identified by a variation of a COPA analysis and unsupervised hierarchical clustering was performed (FIG. 17(a)) Even in the absence of information concerning patient characteristics (including outcome) several clear clusters were obtained. Remarkably, cluster 8 identified a group of children with a markedly poor outcome (Kaplan Meier plot in FIG. 17(b)).

This genetic analysis is important for several reasons. A current protocol under consideration by COG involves the up front testing of diagnostic leukemic blasts using the gene signature described above to identify a cohort of patients that are almost certain to be unresponsive to current therapies. The long term goal of this COG effort is to identify a new generation of treatment specifically tailored to this extremely high risk group. Approximately 50% of these children would be predicted to have an activating mutation in JAK, and the use of well characterized JAK inhibitors is also in the planning stages. However, all of these patients express a subset of the genes used to identify the cohort, and a targeting mechanism dependent only on the presence of the gene products independent of any function would be an ideal approach to new therapies.

We chose CD99 and CRLF from the list of potential cluster 8 targets as initial VLP/protocell targets for multiple reasons. The expression of, either gene alone is predictive of a markedly poor outcome as shown in FIG. 17(b) B, C. Both genes have a well-characterized extracellular domain of 100-125 residues, representing an ideal bait for the identification of binding peptides. In addition, there are suggestions that targeting cells that express either gene will both be tolerated by animals and have applications in diseases other than pediatric ALL.

CD99 is expressed at high levels in multiple tumor types, including Ewing's sarcoma. Antibody-based targeting of CD99 has developed in animals and has been well tolerated, suggesting that the depletion of nonmalignant cells that are CD99 positive is not a major issue. Expression of CD99 is also elevated in cells infiltrating atherosclerotic plaques, and vaccination of mice against CD99 provided protection against plaque formation without major side effects despite the long term loss of CD99 positive lymphocytes and monocytes.

Although CRLF2 (also termed TSLPR) appears to play a minor role in embryonic hematopoiesis, a genetic knockout does not display a phenotype, suggesting that CRLF2 positive cells are not required for critical functions after birth. Alterations in CRLF2 signaling have been postulated to play a major role in aberrant inflammatory responses such as acute dermatitis and asthma, and a significant effort is underway to find small molecule inhibitors of CRLF2 function or compounds to deplete CRLF2 positive cells in patients with severe allergic disorders.

We have cloned both CD99 and CRLF2 in retroviral based expression systems, infected cultured cells that lack endogenous expression, and selected stable transfectants. Cells infected with the CRLF2 virus express very high levels of the protein that is properly trafficked to the membrane, since it is accessible to extracellular antibodies. Similar results have been obtained with CD99. These cells will allow us to take a novel approach to the identification of targeting peptides. Rather than performing differential screens with normal and malignant cells as is often done, we can use cells that differ only by the expression of a single gene product that we have shown to be differentially expressed on the surface of the cell that is to be targeted. We have also made constructs containing the extracellular domain of both CRLF2 and CD99 fused to GST, allowing for a bead based selection strategy.

Taken together these observations suggest that pediatric ALL is an optimal model system for the nano-based targeting experiments. We have used gene expression arrays to characterize a cohort of pediatric ALL patients with a dismal outcome despite intensification of state of the art therapies. We have identified a specific set of proteins with extracellular domains expressed in the blasts of these patients, and propose that a novel approach in which cytotoxic reagents are delivered to cells based on the differential expression of these proteins may markedly improve their survival. We also argue that based on prior studies, targeting of cells expressing either CD99 or CRFL2 will have a minimum of side effects, and may well have important implications for other diseases.

### Example 4

### Development of Targeting Ligands

CRLF2.

CRLF2 may be targeted using its natural ligand TSLP or with peptides that are specifically directed towards CRLF2; both approaches are being developed. To identify CRLF2-specific targeting peptides, we have used a commercial M13 filamentous phage library and our new nanotechnology platform method (MS2 virus-like peptide (VLP) displays) to screen for peptides against Ba-F3 cells (a murine IL-3-dependent pro-B cell ALL cell line) engineered to stably express human CRLF2. Peptides selected by affinity for Ba-F3-CRLF2 cells were counter-selected against parental Ba-F3 cells to eliminate any phage binding receptors common to both cell types. We find that a matched selection/counter-selection pair greatly increases the specificity of the affinity selection process. It is important to note that in the VLP screening method, each VLP encapsidates its own mRNA. This means that the nucleotide sequences encoding any particular VLP and its guest peptide or protein are contained within the particle itself and can be recovered by reverse transcription and polymerase chain reaction. Amplification and re-cloning of the selected sequences leads to the identification of peptide ligands specific for the target.²³ Through this approach, 12 CLRF2 targeting peptides were identified which caused the filamentous phage selectants to bind cells at nanomolar affinities; their specificity for CRLF2 was further demonstrated by their ability to bind the purified protein in vitro (data not shown). Affinity selections conducted in our laboratories have identified a peptide ligand to CRLF2 (TDAHASV) (FIG. 18), demonstrating a Kd of 27.9 nM with no significant binding to the BaF3 parental line (Kd of <3 μM)). This targeting peptide has already been conjugated to protocells and we have demonstrated selective binding and toxicity in CRLF2-expressing ALL cell lines

Targeting CD19 with Single, Chain Variable Region Antibody Fragments (scFV).

Monoclonal antibodies directed towards B cell-specific cell surface antigens (such as CD 19, CD20, or CD22) represent an additional source of targeting agents that can be exploited for nanotherapeutic approaches against a broad range of B cell malignances. Compared to peptides, antibodies offer the prospect of high-affinity binding even when presented at low valency on nanoparticles. We will use our established methods to develop CD19-targeted protocells by conjugating to protocells the single chain variable fragment (scFv) derived from FMC63 anti-CD 19 which has already been successfully used to target CD 19+ cells in murine xenograft models and in human immunotherapy clinical trials for CLL, and more recently, ALL. CD19 is a type I transmembrane glycoprotein of the immunoglobulin Ig superfamily with B cell-restricted expression. As CD 19 is expressed in the earliest (early pre-B cells) to the latest (plasma cells) stages of B cell development, it is an attractive target for therapy of a broad range of B cell malignancies. Numerous B cell-specific anti-CD 19 biologics, including immunoconjugates, have demonstrated efficacy in xenograft models and in human clinical trials for various B cell malignancies. Although CD 19 is efficiently internalized in B cells and is more consistently expressed as a target in pre-B ALL, some investigators believe that CD22 may be a better therapeutic target due to its more rapid internalization. Should CD19-targeted protocells be less than optimally internalized, we will consider the development of CD22-targeted protocells using scFv such as those derived from RFB4.

### Example 5

### CRLF2-Targeted Protocells

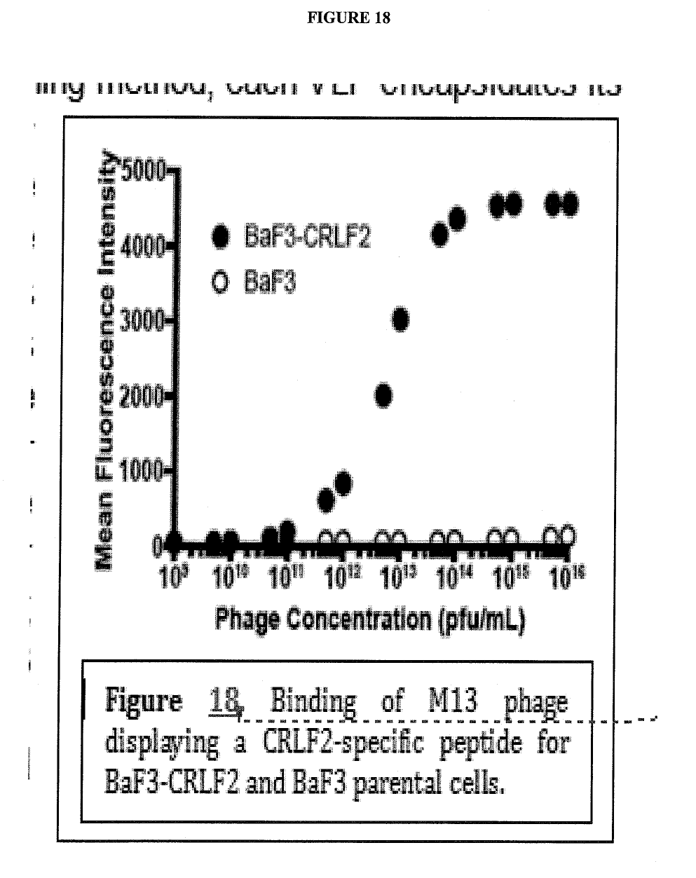

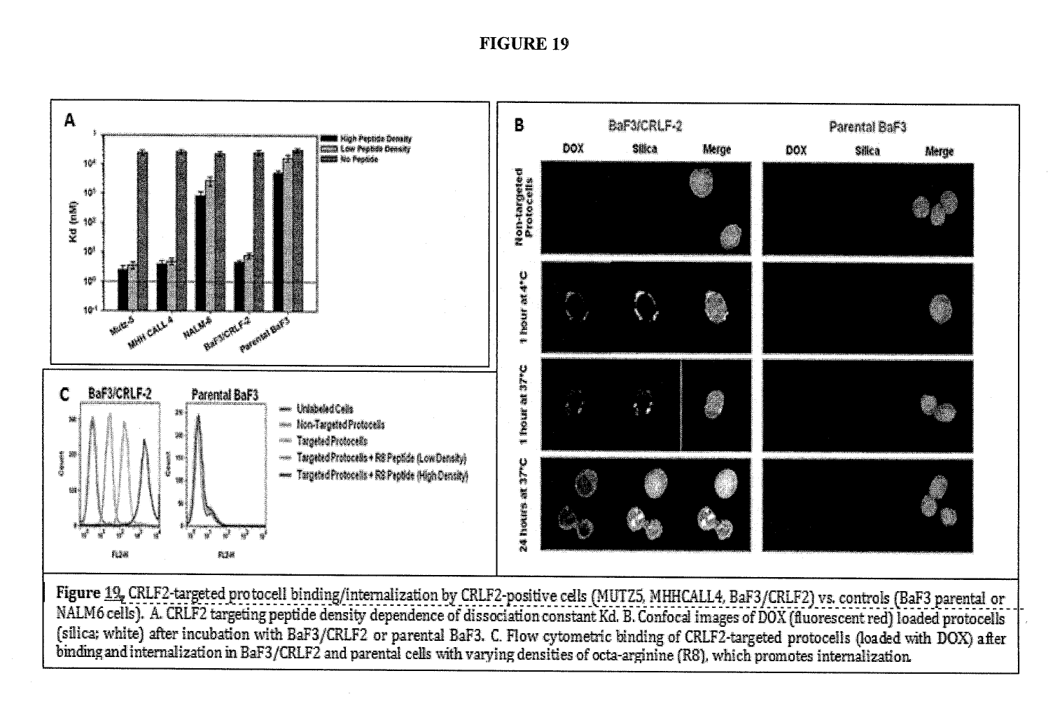

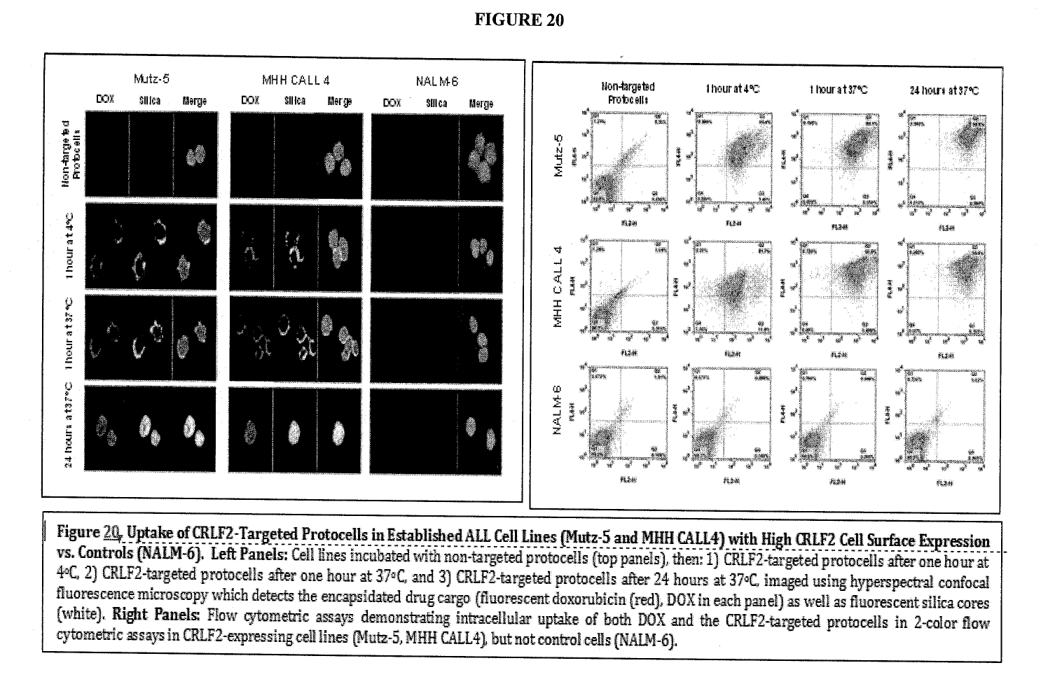

We synthesized CRLF2-targeted protocells by conjugation of the CRLF2-targeting peptide TDAHASV to protocells. CRLF2-targeted protocells were demonstrated to possess a 1000-fold higher affinity for engineered BaF3-CRLF2 cells expressing high levels of CRLF2 (FIG. 19) and for the MUTZ5 or MHHCALL4 cells (FIG. 19, 20) (established human ALL cells lines with CRLF2 genomic rearrangements producing high levels of cell surface CRLF2 proteins and JAK tyrosine kinase mutations), when compared to untargeted protocells, the parental BAF3 cell line, or the CRLF2(−) CD19-positive NALM6 B-precursor ALL cell line, which served as controls. This affinity was also achievable at very low peptide densities (FIG. 19A) due to the fluid protocell surface, potentially minimizing non-specific binding and/or immune responses. Targeted protocells loaded with DOX (which is intrinsically fluorescent) were able to selectively bind to cells expressing CRLF2, and after incubation at 37° C., to become internalized and deliver drug to the cytoplasm of the cells within 24 hours, while showing no non-specific interactions with control cells (FIGS. 19B, 20). Further, modification of the protocell surface with an octa-arginine (R8) peptide promoted this selective internalization in a density-dependent manner (FIG. 19C), proving that protocells support complex synergistic interactions enabling targeting and internalization for cancers whose targeting peptides might be poorly internalized. These preliminary studies demonstrate that we can selectively target CRLF2-expressing ALL cells with CRLF2-targeted nanocarriers in vitro, and, that the protocell and its cargo are internalized and taken up by the cytoplasm.

### Defining Optimal Therapeutic Cargos.

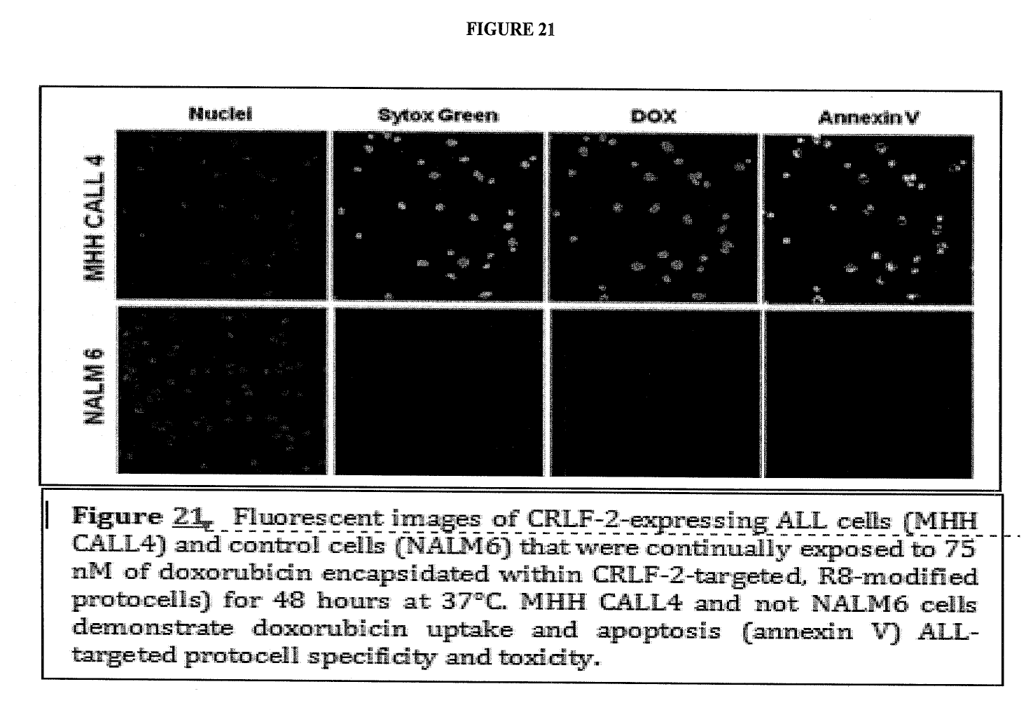

The ability of protocells to protect their therapeutic cargo until released within the target cell and to deliver multiple cargoes is being exploited initially in vitro to determine the most efficacious drug combinations for packaging into ALL-targeted protocells. As shown in FIG. 20, when CRLF2-targeted protocells with encapsidated DOX were incubated with the established MHHCALL4 ALL cell line (with CRLF2 genomic rearrangements and high CRLF2 expression on the cell surface), binding, protocell and drug uptake, and DOX release into the cytoplasm could be demonstrated in CRLF2-expressing cells but not in controls. Although high-risk ALL patients tend to be resistant to intensive therapeutic regimens, we have shown in preliminary studies that after uptake and drug delivery, CRLF2-targeted protocells with encapsidated DOX promoted rapid apoptosis and cell death in MHH CALL4 cells (FIG. 21). Using the established and engineered ALL cell lines described above and in Aim 2, we are testing traditional ALL therapeutic drug combinations as well as novel compounds that we have demonstrated are effective against high-risk ALL (such as the signal transduction inhibitor rapamycin which we have shown is synergistic with DOX (see FIG. 19 in Core D and associated discussion). The therapeutic efficacy of drugs encapsidated in targeted protocells is being compared to exposure of the cell lines to targeted protocells lacking therapeutic cargos, non-targeted protocells (with and without cargos) and free drug(s) using cell biologic, flow cytometric, and phosphoflow cytometric assays (in Core C), allowing us to test and model pharmacodynamic assessments of target inhibition in ALL cells in vitro. The amount of drug carried per protocell is tunable (ranging from 0-50% by weight) and can be modified by changing the concentration of drug in the loading solution. The optimal concentrations of therapeutic cargos will be defined for each drug and drug combination through iterative interactions between in vitro and in vivo.

In our initial studies with DOX, we wish to deliver an equivalent dose via targeted protocells as we will for free drug, in an appropriate therapeutic dose range, in vitro and in the ALL xenograft models in vivo. Thus, the initial loading dose for DOX in protocells will be at 10% protocell particle weight in order to be equivalent to the planned injected dose of free drug (0.2 mg per mouse at 2 mg particles). These studies will include comprehensive dose-response curves using the agents alone and in combination with drugs used in standard protocols, as we have previously published and as detailed further in Core D. The end points will be growth assays as well as biochemical and flow cytometric measurements of apoptosis/necrosis measured at a number time points to determine both early and late effects. Preliminary experiments have validated the cytotoxic efficacy of some of these compounds, although their potency appears low in some cases.

### In Vivo Imaging.

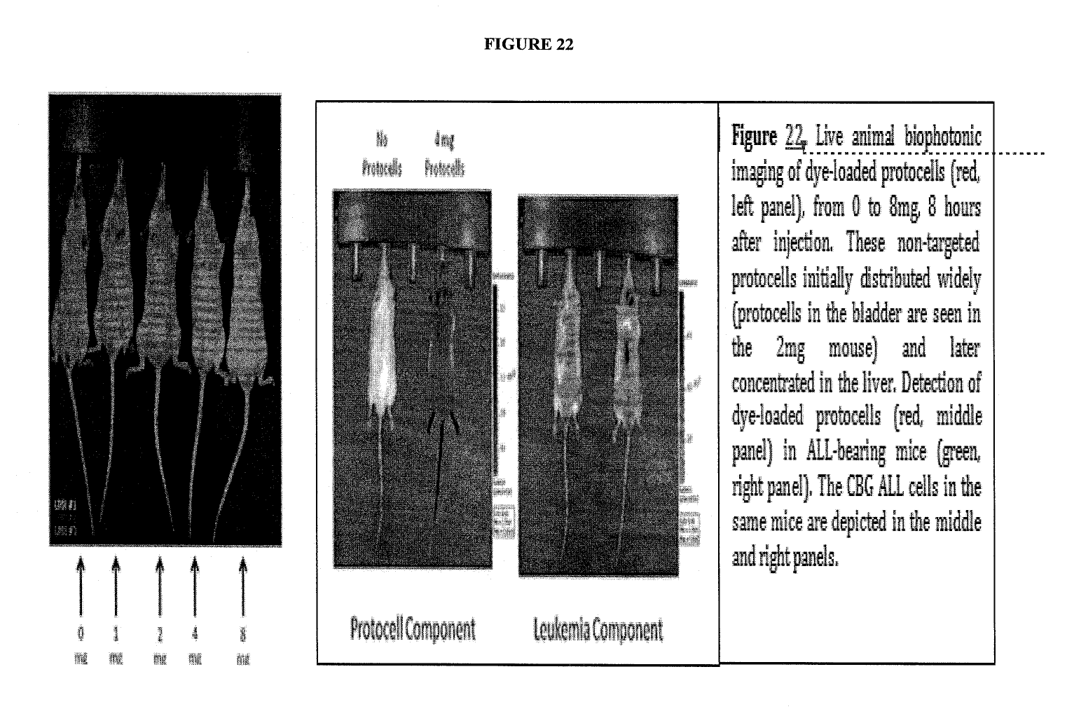

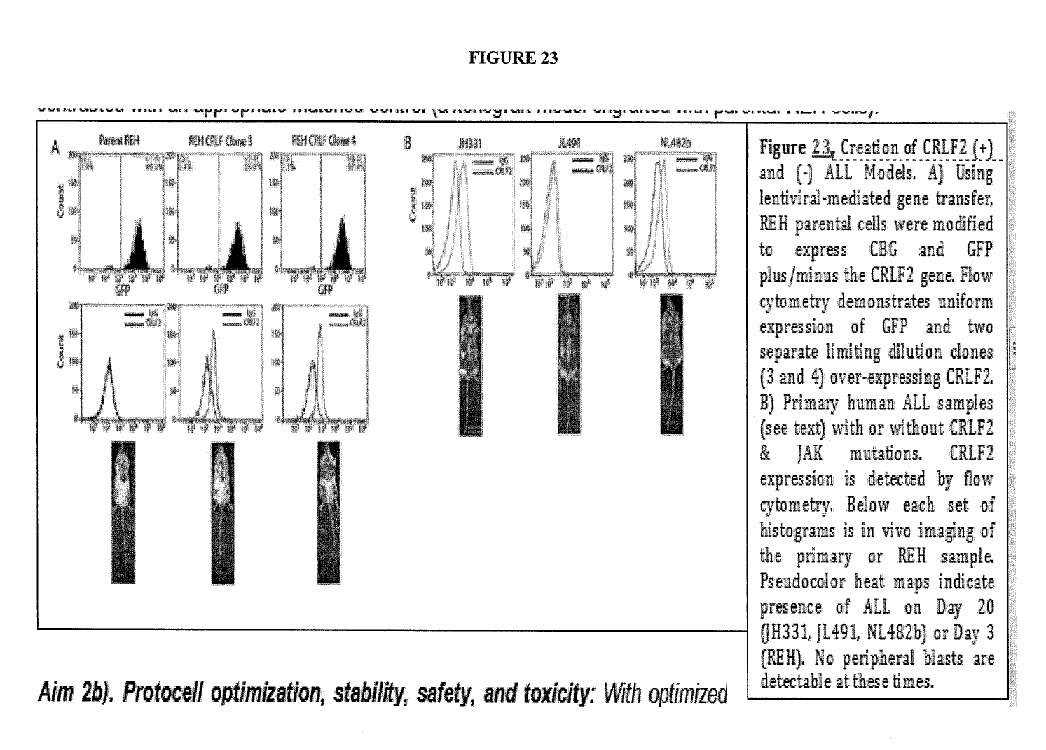

The ability to simultaneously image the bio-distribution and co-localization of cancer cells (such as ALL cells) and a therapeutic (such as T cells or our targeted protocells), has been hampered by the lack of multicolor luciferases with a narrow enough emission spectra to allow spectral un-mixing using the newest generation of optical imaging systems. As detailed in Core D, we have developed a system to overcome this barrier, using click beetle green (CBG) and click beetle red (CBR) luciferases that emit in distinct parts of the spectrum with minimal spectral overlap. Using the innovative imaging modalities in Core D, alone or co-registered with CT, and this novel two-color biophotonic imaging system, we will use CBG luciferase-labeled ALL cells and quantum-dot (Qdot) or dye loaded ALL-targeted protocells to simultaneously assess ALL disease burden, protocell trafficking, protocell/ALL co-localization, and ultimately therapeutic efficacy in vivo in the ALL xenografted animals. Photon intensity scales directly with cell number and can be used to assess disease burden and therapeutic response. Our current focus is on CRLF2 and we are modeling the bio-distribution and co-localization of non-targeted and CRLF2-targeted protocells labeled with Qdots or fluorescent dyes (FIG. 22). In our first in vivo experiments, we encapsidated a far-red fluorescent dye (AlexaFluor 680) in non-targeted protocells and were in fact able to distinguish a red protocell signal distinct from the CBG+ ALL cells (FIG. 22). As we continue these studies with ALL-targeted protocells, we will utilize Qdot technology, as Qdots are very bright, have excellent tissue penetration, and very sharp emission peaks, making them ideal as the second color in our biophotonic in vivo imaging system. Our preliminary experience suggests a Qdot 705 would be ideal, depending on size and protocell loading considerations. We will initially test ALL lines (as detailed in FIG. 23) and then validate our findings using CBG+ primary human ALL xenografts. We will look for ALL/ALL-targeted protocell co-localization and correlate those findings with therapeutic efficacy (Aim 2c). Iterative interactions between Aim 1 and Aim 2 will allow us to determine how protocell modification influences nanoparticle trafficking in vivo, allowing us to optimize the final protocell design (see Table 1, Aim 2b), as needed, to improve trafficking and co-localization with ALL. In addition to the 18 new primary human high-risk ALL xenograft models that we have created with a spectrum of CRLF2/JAK mutations, we have prepared new CBG/GFP-labeled human ALL cell lines and we have established ALL xenografts for in vivo studies from these lines (FIG. 23).

### In Vivo Toxicology.

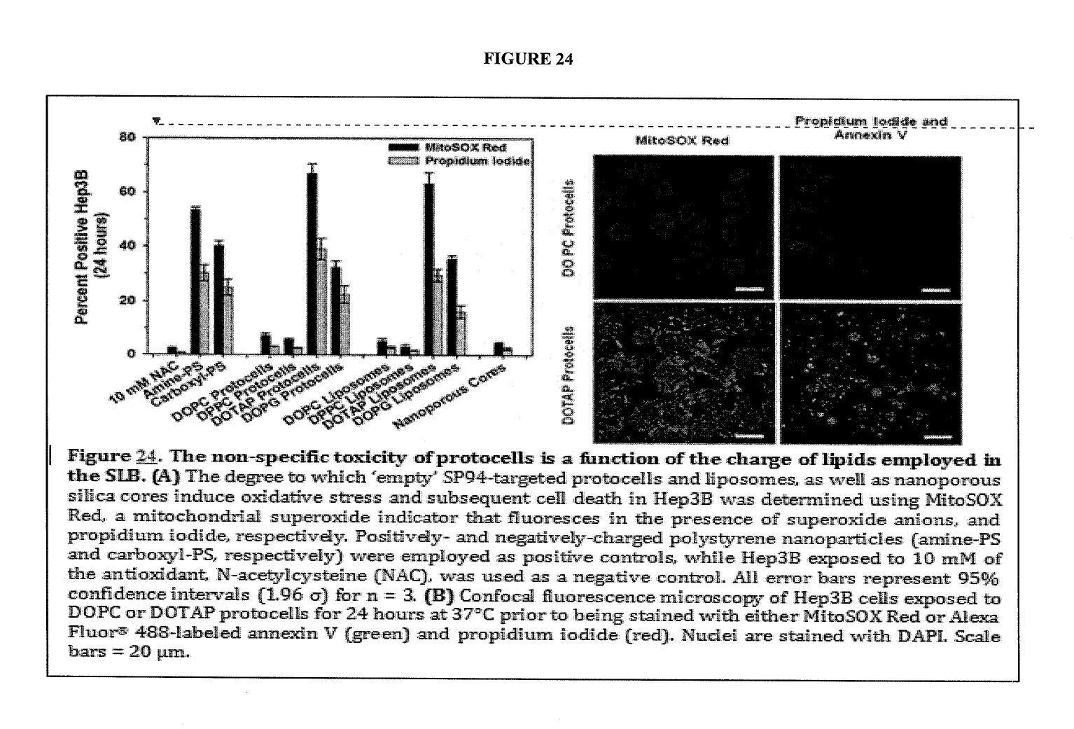

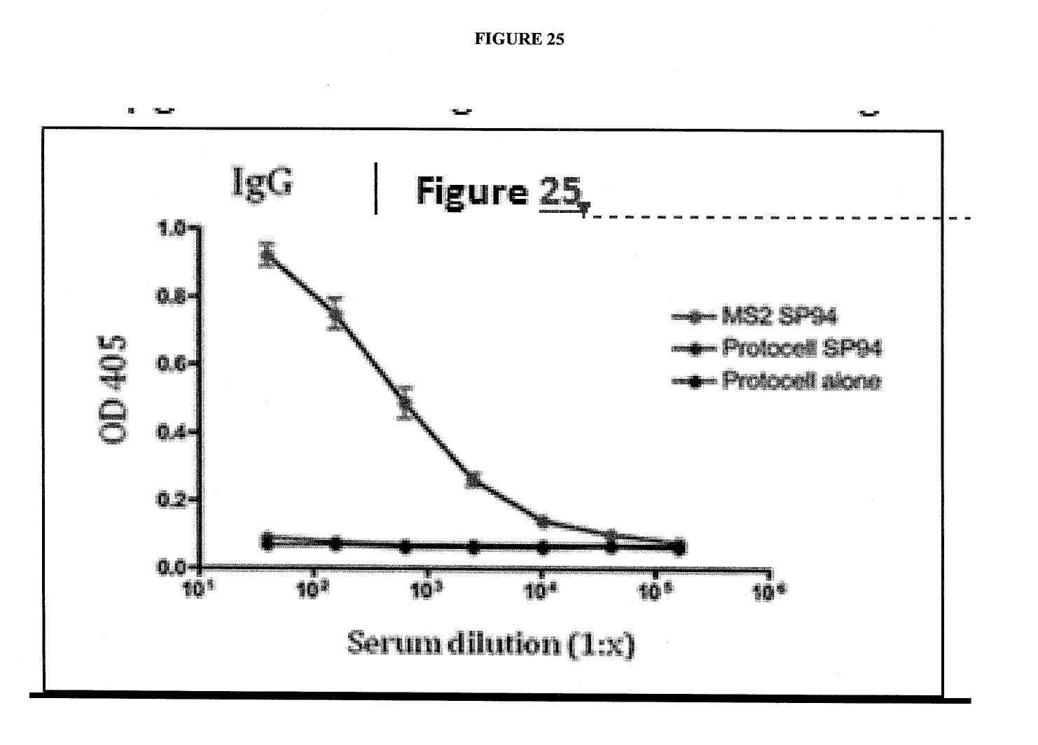

For toxicology studies there are two major issues which must be addressed: silica loading in the tissues, derived from the mesoporous silica core of the protocell, and non-targeted or “off target” delivery of the therapeutic cargos to normal tissues. In each case, our preliminary data suggest that the liver will be the principal target. Nevertheless, we will systematically evaluate all major organs and tissues in treated mice for silica deposition and cellular damage after treatment with loaded and unloaded protocells. There are multiple lines of evidence that protocells will have low and acceptable toxicity profiles in vivo: 1) silica is accepted as “Generally Recognized As Safe” (GRAS) by the US Food and Drug Administration (FDA); 2) recently, solid, dye-doped silica nanoparticles have received approval from the FDA for targeted molecular imaging of cancer; 3) compared to solid silica nanoparticles, mesoporous silica nanoparticles exhibit reduced toxicity/hemolytic activity due their surface porosity lowering the contact area of surface silanols with cell membranes; 4) in the case of protocells, the supported lipid bilayer reduces silica/membrane interactions and confers safety profiles and immunological behavior comparable to liposomes (FIG. 24); and 5) the high internal surface area (>1000 m²/g and ultra-thinness of the silica walls (<2-nm) of the porous silica core result in a high dissolution rate and soluble silica, e.g. Si(OH)₄, has extremely low toxicity. As a benchmark, we compared oxidative stress and cell viability of neutral, positively and negatively-charged targeted protocells with corresponding liposomes and other control particles at 10⁹ particles/mL corresponding to ˜1-2 μg/mL (FIG. 24). The targeted, zwitterionic DOPC protocells, which are the focus of our proposed studies, show minimal effects on viability and reactive oxygen species (ROS) generation. In preliminary studies (FIG. 25), we also compared the IgG response of C57Bl/6 mice immunized twice with 10 μg doses of targeted and SP94-targeted protocells to that of virus-like particles (VLPs) conjugated to the same concentration of targeting peptide (MS2 SP94). Targeting peptides conjugated to lipid bilayers elicit only a weak response, because they do not support T cell help needed for higher affinity IgG, whereas targeting ligands displayed on VLPs induce a strong IgG response. When considering therapeutically administered doses, for the hepatocellular carcinoma cell line Hep3B, the LC50 and LC90 values of free DOX are 150 and 500 ng/mL, respectively. As we have demonstrated, when using targeted protocells, these values fall to 6 ng/mL and 20 ng/mL due to the protocell capacity, stability, and internalization efficiency. If only a few percent of protocells are delivered to the ALL target, then the needed administered doses of protocells are less than about 100 μg/ml, where numerous studies have shown insignificant toxicity. To assess organ damage after novel agent treatment, organs from treated and control mice will be harvested and fresh frozen or fixed in formalin for histopathology. As discussed above, Si loading will be assessed by ICP-MS. If abnormalities are identified, we will use tissue specific stains and electron microscopy to determine the underlying pathophysiologic effects of the protocells. We will compare the toxicity of targeted and non-targeted protocells and compare protocells with encapsidated DOX with intravenous DOX provided as free drug at an equivalent dose (typically 0.2 mg/mouse). We will also assess if there is different toxicity in mice xenografted with hepatotropic Nalm-6) and non-hepatotropic (380) ALL cell lines. As the liver is the principal nonspecific target organ of the protocells, we will assess hepatocellular damage with serum transaminases (ALT, AST), cholestasis with bilirubin, and liver function with prothrombin time testing⁵⁰ and albumin before and 1-7 days after exposure to nanoparticles at single and weekly doses. In addition, we will assess the hematologic toxicity of DOX-loaded and unloaded protocells. Based on our prior studies evaluating the toxicity of different nanoparticle delivery systems, 20 mice will be needed for multi-compartment toxicity analysis in each condition with each mouse strain (see Vertebrate Animals and Core D). We will expand this number if needed. We will not only assess toxicity in the NSG xenografts, but also in other strains including BALB/-C, Emu-ret, Rag1ko, and MRL-lpr, allowing us to determine if the toxicity of protocells is different in immune competent, immune deficient, and/or in mice with activated immune systems due to autoimmune disease.

### Example 6

### Combinations of Peptides can be Used to Direct Targeting and Internalization for Non-Internalized Receptors

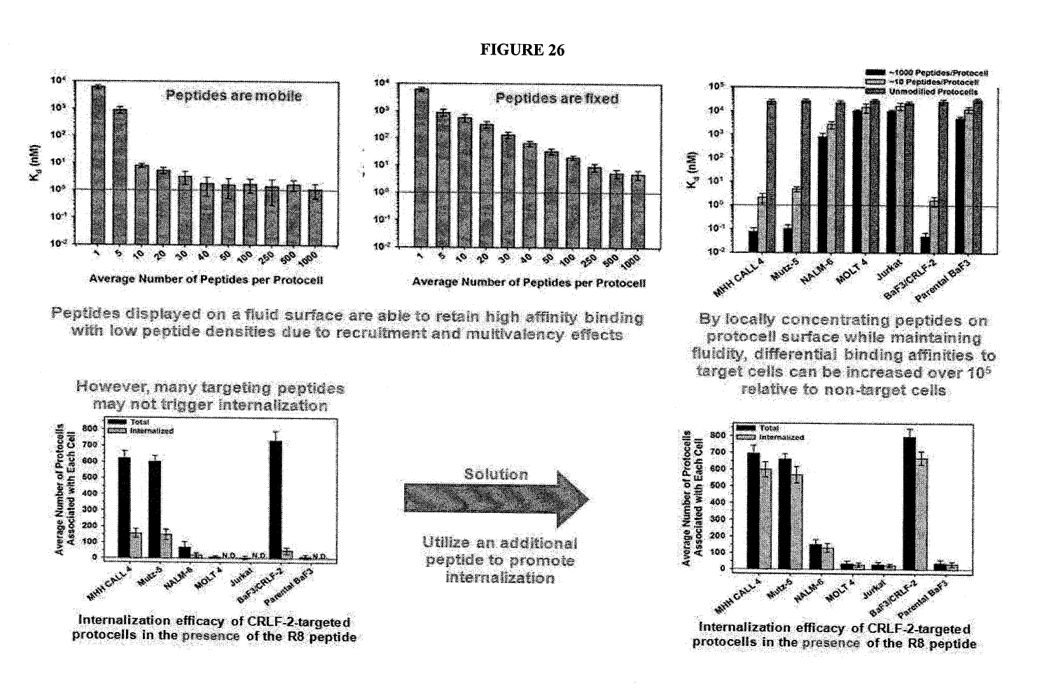

As shown in FIG. 26, peptides displayed on a fluid surface are able to retain high affinity binding with low peptide densities due to recruitment and multivalency effects By locally concentrating peptides on protocell surface while maintaining fluidity, differential binding affinities to target cells can be increased over 105 relative to non-target cells. However, many targeting peptides may not trigger internalization. The solution to this problem is to utilize an additional peptide to promote internalization. FIG. 26 shows Internalization efficacy of CRLF-2-targeted protocells in the presence of the R8 peptide.

### Example 7

### Biophotonic Imaging in Murine ALL Xenograft Model

Protocells comprised of fluorescent label and made in accordance with the technique described below were tested in a murine luminescent leukemia model, as illustrated in FIGS. 27-29.

Our discovery of these novel ALL subtypes, together with our preliminary studies demonstrating a lack of efficacy of JAK kinase inhibitors as single agents in our xenograft models of human ALL containing CRLF2 and JAK mutations, and the observation that a large percentage of high risk B-precursor ALL samples express measurable levels of CRLF2 mRNA compared to normal B cells and respond to TSLP, leads us to hypothesize that CRLF2 is a superior target for therapy in high-risk ALL. In order to expand the universe of potential molecular targets with a parallel increase in leukemic subtypes that are amenable to treatment, as well as to allow for simultaneous targeting with multiple classes of particles we propose to also build protocells engineered to target molecules expressed on a wider class of ALL blasts and B cell malignancies, including CD19 and CD22.

Included in these are primary human ALL samples from patients in the “kinase-active” group of ultra-high risk ALL patient. Collaborating with COG and the NCI TARGET initiative, we received 21 ALL samples in each of 4 combinations of CRLF2 high/low and JAK2 mutant/normal. Eighteen of these 21 samples were established as re-transplantable ALL xenografts in highly immunodeficient NSG (NOD-SCID/gamma common knockout) mice. We successfully created xenografts from each of the 4 subtypes, allowing expansion of these cells for in vitro and in vivo studies. This is the approach we have used successfully in testing signal transduction inhibitors, including mTOR inhibitors, as well as engineered anti-ALL (CD 19-directed) T cell therapy, as ALL therapeutics. 13

These have been studies with strong mechanistic analysis but also with a clear translational focus. The second area of innovation concerns the development of novel in vivo imaging approaches in xenograft models. Firefly luciferase is widely and successfully used to detect cells using biophotonic imaging in the live animal and is a powerful approach to assess disease burden and to image anatomic localization of labeled cells. In systems where the experimental question is colocalization of cancer cells and a therapeutic such as T cells or nanoparticles, an approach which allows simultaneous detection of two different cell or particle types would be an important methodological advance. This has been hampered by the lack of multicolor luciferases with a narrow enough emission spectra to allow spectral unmixing using the newest generation of optical imaging systems (spectral unmixing is conceptually similar to compensation in flow cytometry). We have recently developed a system to address this, using click beetle green and click beetle red luciferases (CBG and CBR) that emit in distinct colors with minimal spectral overlap. An example of the power of this approach is shown in FIGS. 27-29, where engrafted human ALL cells and human T cells expressing CBG and CBR are both separately and simultaneously imaged in the living mouse. Photon intensity scales directly with cell number and can be used to assess disease burden and response (FIGS. 27-29).

### Example 8

### Virus-Like Particles (VLPs) of Bacteriophage MS2 for Selection of Peptides that Bind to ALL-Specific Targets

We have generated random peptide libraries displayed on VLPs of the icosahedral RNA bacteriophage MS2. Caldeira J, Peabody D. 2007. Stability and assembly in vitro of bacteriophage PP7 virus-like particles. Journal of Nanobiotechnology 5: 10 Peabody D S, Manifold-Wheeler B, Medford A, Jordan S K, do Carmo Caldeira J, Chackerian B. 2008. Immunogenic display of diverse peptides on virus-like particles of RNA phage MS2. Journal of Molecular Biology 380: 252-63). In general, phage display depends on (i) the ability to genetically fuse peptides to a viral structural protein so that they are presented in an accessible form on the surface of the viral particle and (ii) the specific encapsidation of the nucleic acid that encodes the peptide-protein fusion, which provides a means to amplify affinity-selected sequences. Here we briefly describe how we have genetically engineered the MS2 VLP to display diverse peptides and encapsidate the mRNAs that encode them before describing proposed affinity selection experiments.

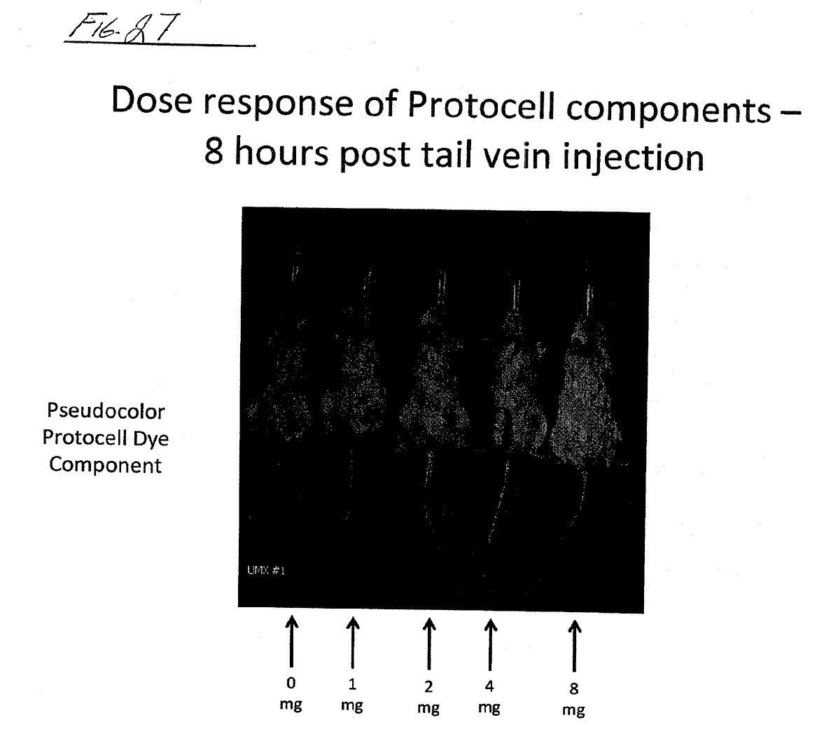

The MS2 capsid is composed of 90 coat protein dimers that, when expressed from a plasmid in E. coli, spontaneously self-assemble into an icosahedral shell that is 27.5-nm in diameter. Since the wild-type dimer does not generally tolerate peptide insertions, we genetically fused the C-terminus of the upstream monomer to the N-terminus of the downstream monomer so that both halves of the dimer are produced as a single polypeptide. We have found that the resulting ‘single-chain dimer’ (sc-dimer), tolerates >90% of randomized 6-mer, 8-mer, and 10-mer inserts and yields properly assembled VLPs, each of which displays 90 copies of a foreign peptide on its surface. Id. We have also demonstrated that each VLP encapsidates its own mRNA. Id. This ensures that the nucleotide sequence that encodes a recombinant VLP is contained within the particle itself and can be recovered by reverse transcription and polymerase chain reaction (RT-PCR), making possible the affinity selection scheme illustrated in FIG. 30.

In the ‘VLP display’ process, random sequence libraries are subjected to selection against the target molecule or cell, and amplification and re-cloning of the selected sequences leads to identification of peptide ligands specific for the target. Chackerian B, Caldeira Jd C, Peabody J, Peabody D S. 2011. Peptide Epitope Identification By Affinity-Selection On Bacteriophage MS2 Virus-like Particles. Journal of Molecular Biology In Press, Accepted Manuscript; Carnes E C, Lopez D M, Donegan N P, Cheung A, Gresham H, Timmins G S, Brinker C J. 2010. Confinement-induced quorum sensing of individual Staphylococcus aureus bacteria. Nat Chem Biol 6: 41-5.

To facilitate library construction and screening, we constructed a plasmid (pDSP62) that expresses high levels of MS2 coat protein from the bacteriophage T7 promoter. This vector normally replicates using a ColE1 origin but additionally contains a M13 origin so that a single-stranded version of the plasmid can be produced after super-infection with a M13 helper phage. This enables production of complex random sequence libraries via in vitro extension of mutagenic primers on circular single-stranded templates using an efficient mutagenesis procedure. To restrict insertion of peptides to the AB-loop of the downstream half of the sc-dimer, the upstream copy is a synthetic ‘codon-juggled’ coat sequence containing the maximum possible number of silent mutations. Thus, mutagenic primers can be targeted to anneal specifically to the downstream site. Using this vector, random sequence 6-mer, 7-mer, 8-mer, and 10-mer libraries containing >10¹⁰ individual members have been produced. (6) MS2 VLPs normally display 90 peptides per particle, making it difficult to discriminate peptides with high intrinsic binding affinities from those that have low affinity but bind with high avidity by virtue of multiple weak interactions.

To introduce valency control into the MS2 system, we constructed a second vector (pDSP62(am)) that encodes an alternate version of the sc-dimer with a stop codon separating its two halves. This mutant normally produces only wild-type coat protein from its upstream half, but, in the presence of a non-sense suppressor tRNA, a small percentage of ribosomes read-through the stop codon to produce the entire sc-dimer with its guest peptide (the peptide is displayed only in the downstream half). Both the wild-type and sc-dimer proteins are synthesized from a single mRNA, which they encapsidate when they co-assemble into a mosaic VLP that displays, on average, 3 peptides per particle. Using the MS2 VLP system, three or four rounds of affinity selection against antibodies with known epitopes (e.g. the anti-FLAG antibody, M2) yield peptides that closely mimic those epitopes. Chackerian B, Caldeira Jd C, Peabody J, Peabody D S. 2011. MS2 VLP random sequence libraries are subjected to affinity selection to identify peptides that target surface receptors expressed specifically by leukemia cells.

To accomplish this, we have cloned CRLF2 into a retroviral-based expression system, infected cultured cells that lack endogenous expression (BaF3, a murine IL3-dependent pro-B cell line), and selected stable transfectants. Cells infected with the CRLF2 virus express high levels of the protein, which is accessible to extracellular antibodies and is, therefore, properly trafficked to the membrane. Rather than performing differential selections against malignant and normal cells (which we have found results in a large number of peptides that bind to unsuitable targets), we will employ BaF3-CRLF2 cells in positive selections and parental BaF3 cells in counter-selections. We have, additionally, fused the extracellular domain of CRLF2 to GST, which will allow for bead-based selection strategies.

### Development of B Cell-Specific Targeting Antibody Fragments.

Single-chain antibody (scFv) display is generally accomplished by genetically fusing the foreign sequence to the C-terminus of a phage coat protein. However, in the case of MS2 VLPs, the presence of a scFv fusion on every copy of coat protein will likely interfere with capsid assembly. Therefore, in this aim, we will attempt to produce VLPs that display scFvs by inserting a stop codon in between the foreign protein and the C-terminus of coat protein. As described above, addition of a nonsense-suppressing tRNA will cause occasional read-through of the stop codon and production of the fusion protein. Suppression is relatively inefficient, however, so only a few percent of coat protein molecules will contain the C-terminal extension. Co-assembly of wild-type and fusion proteins should produce VLPs with an average of 3-6 scFvs per particle. If this strategy is successful, we will generate a randomized scFv library using the vectors described above and perform affinity selections against CHO cells transfected to express the B cell-specific surface antigen, CD19.

### Example 9

### Development of Targeting Ligands

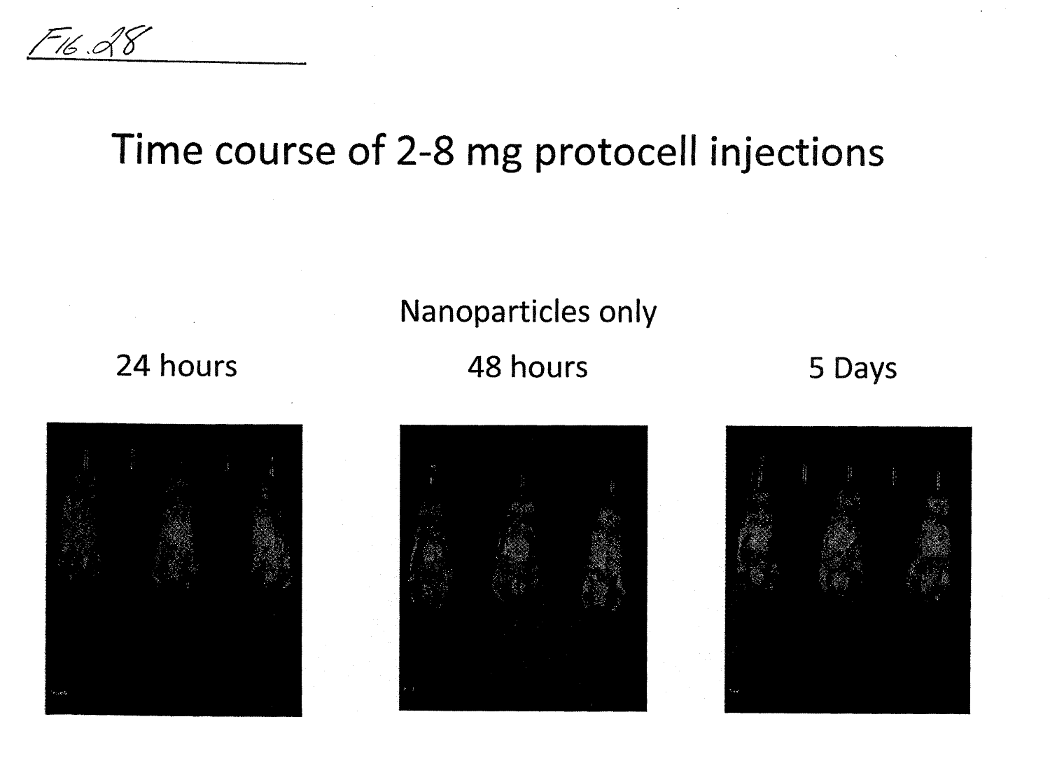

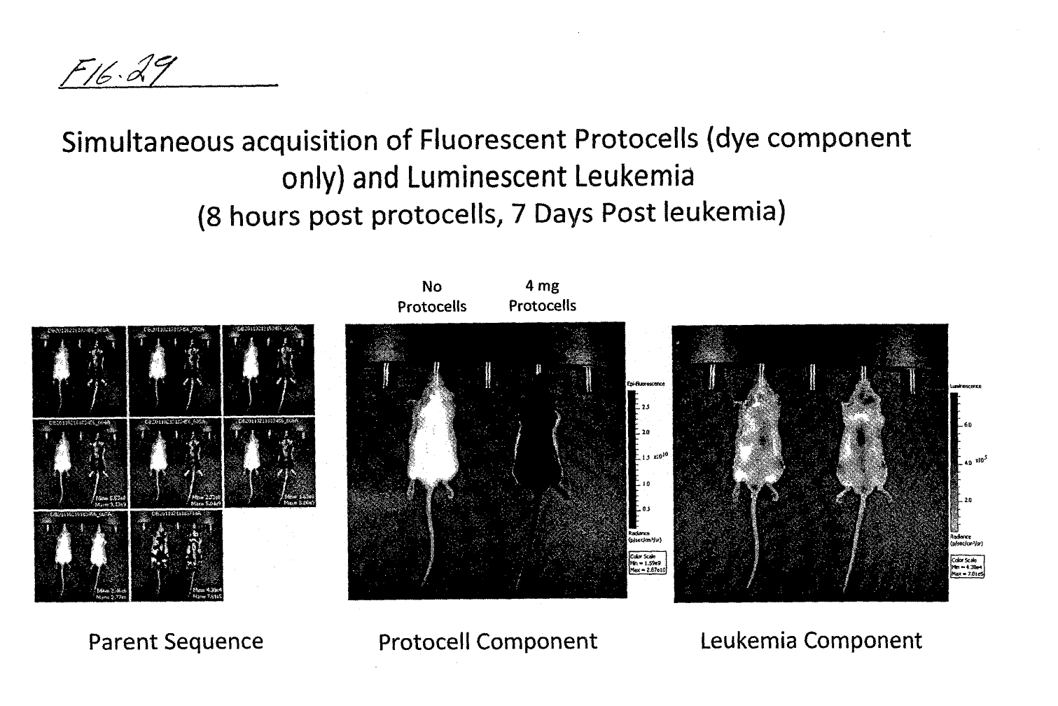

Preliminary proof-of-principle approaches were designed to design and synthesize particles targeting CRLF2, an antigen expressed on a subset of very-high risk pediatric ALLs with a very poor outcome. To identify CRLF2-specific targeting peptides, we have used both commercial M13 peptide libraries and we have developed a novel method—bacteriophage MS2 virus-like peptide (VLP) displays (detailed in FIG. 31)—to screen for peptides against Ba-F3 cells engineered to stably express human CRLF2. Peptides selected by affinity for Ba-F3-CRLF2 cells were counter-selected against parental Ba-F3 cells to eliminate any phage binding receptors common to both cell types, creating an ideal model for counter-selections. We find that a matched selection/counter-selection pair at very high stringency greatly increases the specificity of the affinity selection process.

It is important to note that in this novel method, each VLP encapsidates its own mRNA (FIG. 31) such that the nucleotide sequences encoding any particular VLP as well as the targeting peptide or protein are contained within the particle itself, and can be recovered by reverse transcription and polymerase chain reaction. Amplification and re-cloning of the selected sequences leads to the identification of peptide ligands specific for the target. Using a locally engineered modified pDSP62 system, random sequence 6mer, 7mer, 8mer and 10mer libraries containing more than 10¹⁰ individual members have been produced. The high density of MS2 VLP display (90 peptides per particle) can make it difficult during affinity selection to discriminate peptides with high intrinsic binding affinities from those that have low affinity, but bind with high avidity by virtue of multiple weak interactions, but we have overcome this problem by introducing a valency control in the MS2 system, by making an alternate version of the single chain-dimer with a stop codon separating its two halves; this mutant normally produces only wild-type coat protein from its upstream half, but in the presence of a nonsense suppressor tRNA, a small percentage of ribosomes read through the stop codon to produce the entire single chain-dimer with its guest peptide. Both the wild-type and single chain-dimer proteins are synthesized from a single mRNA, which they encapsidate when they co-assemble into a mosaic VLP that displays about three peptides per particle on average. Through this approach, 12 potential CLRF2 targeting peptides were identified. Their specificity for CRLF2 has been further demonstrated by their ability to bind the purified protein in vitro (data not shown). Affinity selections conducted in our laboratories have identified a peptide ligand to CRLF2 (TDAHASV) (FIG. 31; demonstrating a Kd of 27.9 nM with no showing significant binding to the BaF3 parental line (Kd of <3 μM). As detailed below, this CRLF2 targeting peptide has already been conjugated to protocells and we have demonstrated selective binding and toxicity in CRLF2-expressing ALL cell lines. We have engineered retroviruses that direct the expression of both CD19 and CD22, and will construct T cell lines that express high levels of ectopic protein on their surface. These cells will be used to identify and characterize peptide targeting ligands in a manner identical to what we have demonstrated for CRLF2.

### Single Chain Antibody Fragments (CD19, CD22).

Monoclonal antibodies directed towards B cell-specific cell surface antigens represent an additional source of targeting agents that can be exploited for nanotherapeutic approaches against a broader range of B cell malignances. We have already developed a similar strategy against CD19 to develop therapeutic T cells. Compared to peptides, antibodies offer the prospect of high-affinity binding even when presented at low valency on nanoparticles, and, in many instances detailed knowledge of binding targets and internalization properties are known.

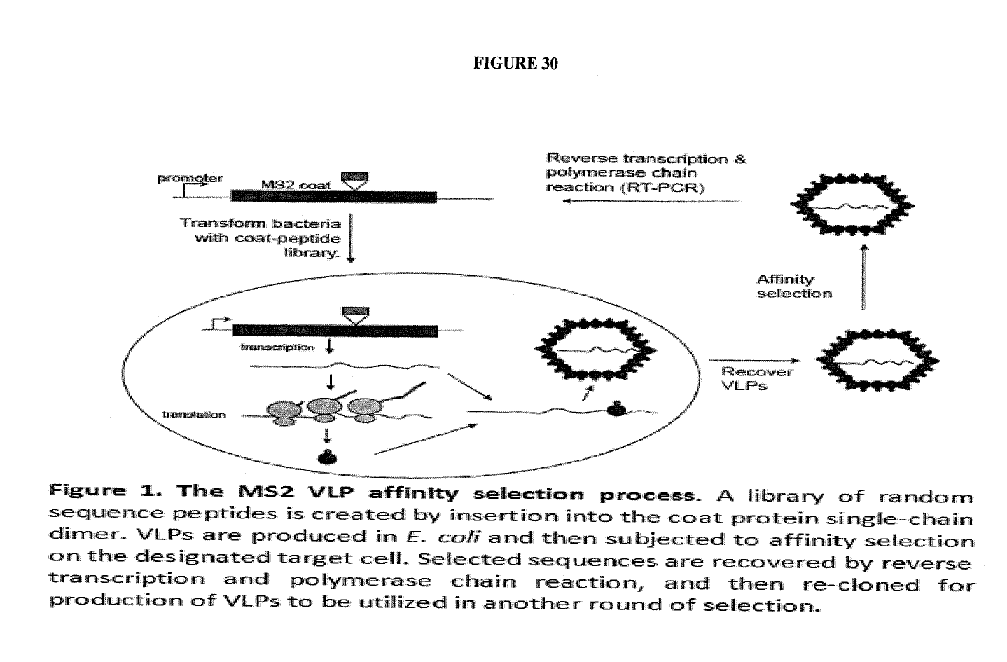

We recently adapted the MS2 VLP^{6,7} for display of antibody fragments by fusing the coding sequence for a single-chain antibody fragment (scFv) to the MS2 coat protein. The particles as designed to display a controlled number of scFv's per particle. We have so far fused several different scFv's to coat protein and demonstrated the ability of the VLP-scFv to bind its target. Based on published amino acid sequences, we synthesized (using assembly PCR) an E. coli codon-optimized DNA sequence that encodes the anti-CD 19 protein and fused it to the C-terminus of the MS2 coat protein sequence with an amber codon at the fusion junction. When this gene is expressed in bacteria with a suppressor tRNA, it produces large amounts of single-chain coat protein, and small amounts (a few percent) of the coat-scFv fusion, which co-assemble to yield a VLP displaying a few antibodies per particle, on average. FACS analysis shows that the CD19-specific scFv directs VLPs to bind CD19+NALM6 B-ALL cells (FIG. 32), but not to cells lacking CD 19 expression. Future studies will characterize the affinity of the interaction and more carefully document its specificity. A similar VLP displaying anti-CD22 has been constructed, and, as with the anti-CD19 particle, will be characterized both for the affinity and the specificity of its interaction with cell lines specifically expressing these antigens. These scFv constructs will then be conjugated to protocells.

Aim 1b. Targeted Protocell Production and Optimization.

Protocell nanoporous silica cores will be synthesized by self-assembly and loaded with cargos by immersion; their supported lipid bilayers will be modified with targeting and fusogenic peptides, single-chain antibodies, and PEG to create sets of targeted nanoparticles. Further optimization will be accomplished by determining cargo content, determining the necessary extent of modification with fusogenic peptides and poly(ethylene glycol) (PEG), pore size, and solubility of the nanoporous silica core (which controls loading and release rates). Protocells will be studied in vitro in ALL cell lines using flow cytometry and hyperspectral fluorescence confocal microscopy.

### Protocell Binding, Specificity, Internalization and Cytotoxicity:

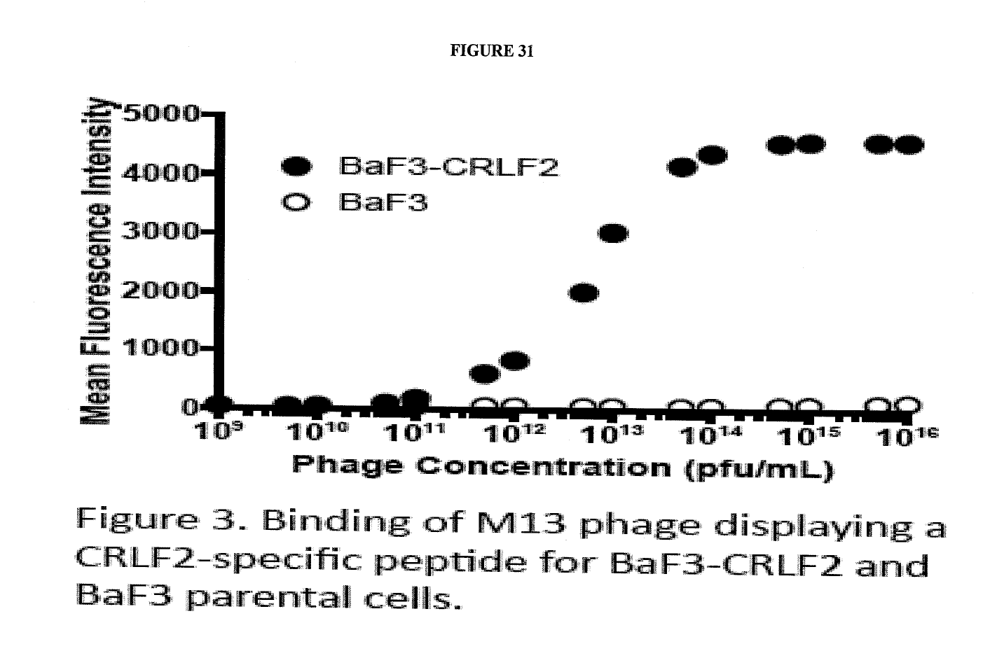

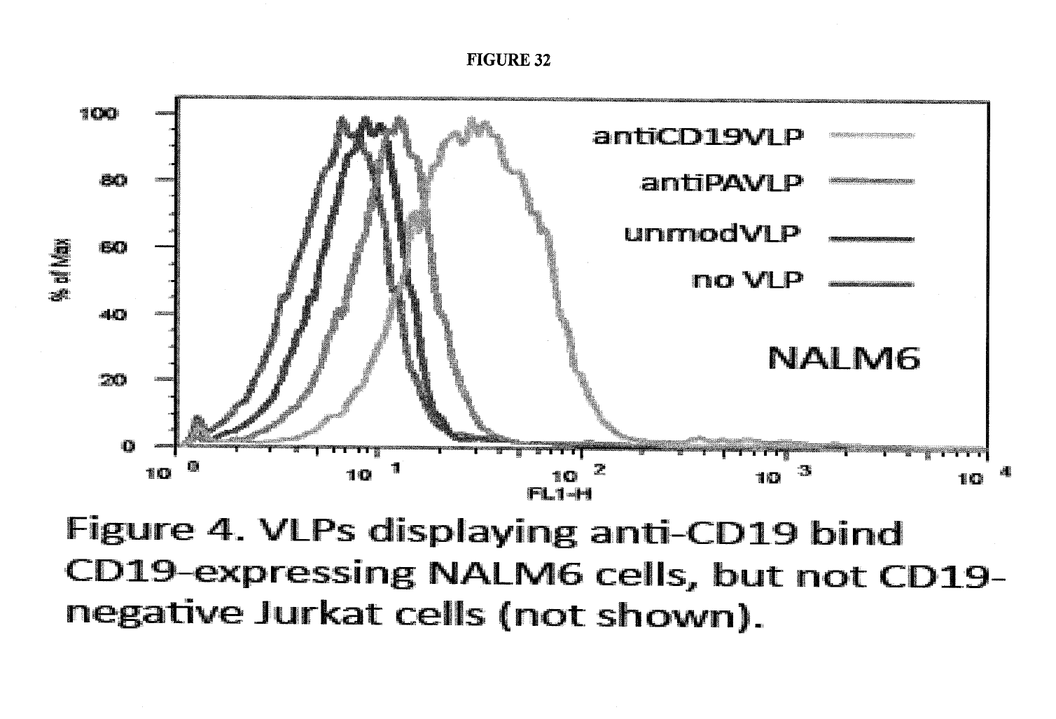

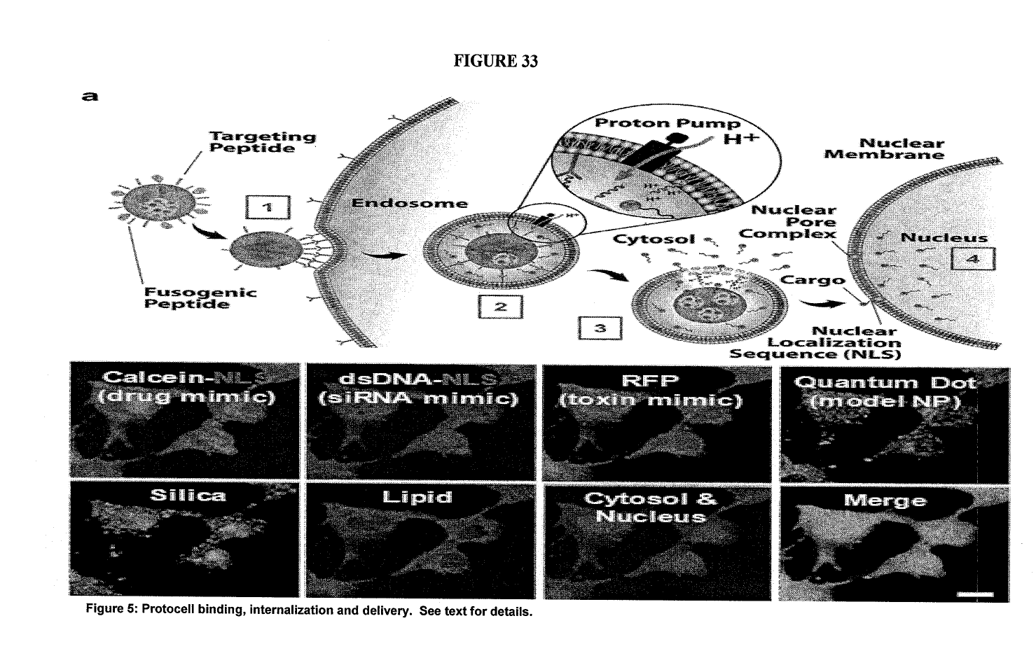

Protocells are synthesized via fusion of liposomes to spherical, nanoporous silica cores (100-150 nm in diameter) that are pre-loaded via simple immersion in a solution of the desired cargos. Based on optimization studies aimed to maximize colloidal stability and cargo retention in simulated body fluids and minimize non-specific interactions with serum proteins and non-targeted cells, we utilized the following supported lipid bilayer (SLB) composition in all surface-binding, internalization, and cargo delivery experiments: DOPC (Tₘ=−20° C.) or DPPC (Tₘ=44° C.) with 5 wt % DOPE 30 wt % cholesterol, and 5 wt % 18:1 (or 16:0) PEG-2000 PE. Using a hetero-bifunctional crosslinker with a PEG (n=24) spacer, SP94 peptides (a targeting ligand specific for hepatocellular carcinoma cells (HCC) identified via filamentous phage display detailed in were covalently conjugated to DOPE moieties in the SLB at concentrations ranging from 0.002-5.0 wt % (corresponding to 1-2048 peptides per particle, on average). 120-nm liposomes with identical bilayer compositions were synthesized for comparative purposes. FIG. 33A depicts the successive stages of protocell binding (step 1), internalization (step 2), endosomal escape (step 3), and nuclear targeting of desired cargo(s) (step 4) by which protocells selectively deliver encapsulated cargos to a cell of interest. Importantly, the fluid but stable SLB enables targeting peptides to be recruited to cell surface receptors. This promotes high avidity multivalent binding and internalization by receptor mediated endocytosis. Dissociation constants (Kd, where Kd is inversely related to affinity) were used to quantify surface binding of SP94-targeted protocells to hepatocellular carcinoma cells (Hep3B), normal hepatocytes, endothelial cells, and immune cells.¹ Protocells modified with only six SP94 peptides per particle exhibit a 10,000-fold greater affinity for Hep3B than for normal hepatocytes, and other control cells (FIG. 34a), providing the specificity necessary for efficacious targeted delivery.¹ Furthermore, SP94-modified protocells have a 200-fold higher affinity for Hep3B than free SP94, a 1000-fold higher affinity for Hep3B than nanoparticles bearing of a non-targeting control peptide, and a 10⁴ higher affinity for Hep3B than unmodified particles (FIG. 34a).

Importantly, the affinity of protocells is a function of both peptide density and the fluidity of the supported lipid bilayer, and the dissociation constant (K_{d}) can be precisely controlled by changing the composition of the bilayer to include varying amounts of fluid and non-fluid lipid components. To demonstrate that binding results in internalization and cytosolic delivery of multiple cargos, FIG. 34b shows hyperspectral confocal images of four categories of fluorescently labelled cargo mimics delivered by a single targeted protocell. After 4 hours, calcein (a drug mimic), ds-DNA (an siRNA mimic), red fluorescent protein (a toxin mimic), and quantum dots are delivered into the cytosol. Calcein and dsDNA (both conjugated with a nuclear localization sequence) are further delivered into the nucleus. Delivery profiles are controlled by the pore size and solubility of the silica core along with protocell surface modification with a fusogenic peptide that promotes osmotic swelling and endosomal disruption (see FIG. 33A, step 3).

FIG. 34b compares the percentage of viable multi-drug resistant Hep3B or hepatocytes after exposure to LC₉₀ or LC₅₀ concentrations of the chemotherapeutic drug doxorubicin (or a drug cocktail) delivered in targeted DOPC (fluid) protocells, DOPC liposomes, or state of the art DSPC (non-fluid) liposomes. For DOPC protocells+DOX, we observe spectacular MDR1⁺ Hep3B-specific cytotoxicity, whereas for corresponding DOPC liposomes+DOX the results are comparable to free DOX. This difference is attributable in part to fluid liposomes being unstable and leaking their cargo. Even stable DSPC liposomes, however, show substantially inferior properties. These data reveal that the combined capacity, stability, and targeting efficacy of the protocell allow it to significantly outperform liposomal delivery agents. In fact a single protocell loaded with a drug cocktail is able to kill a drug-resistant HCC cell, representing a million-fold improvement over comparable liposomes

### CRLF2-Targeted Protocells.

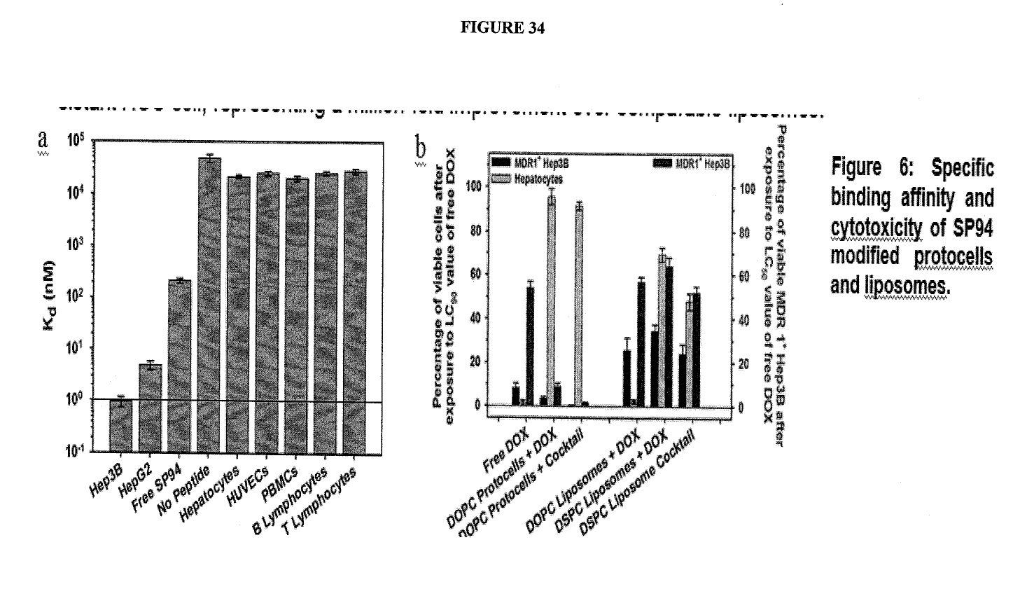

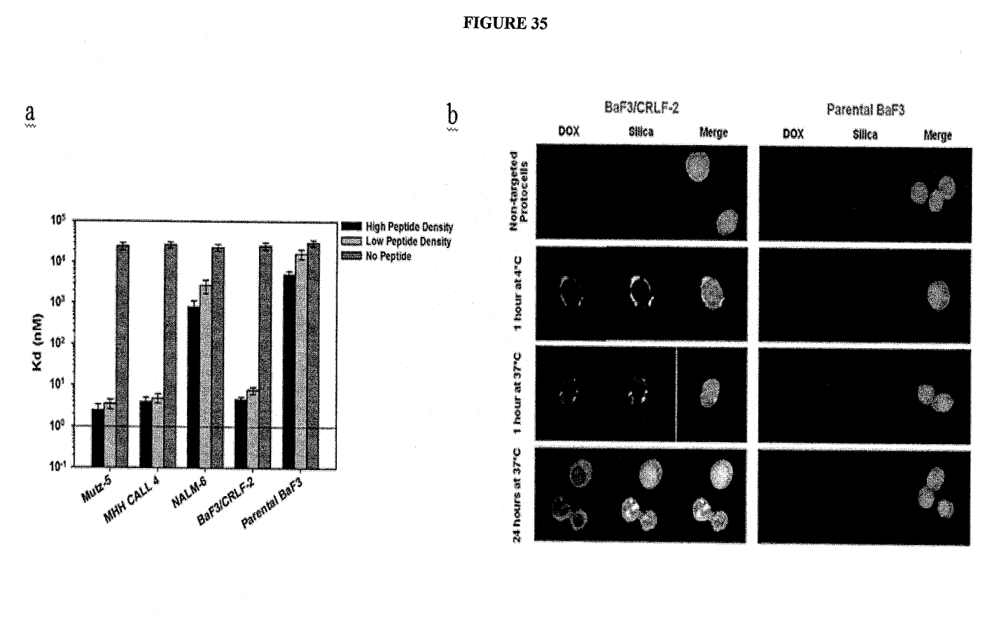

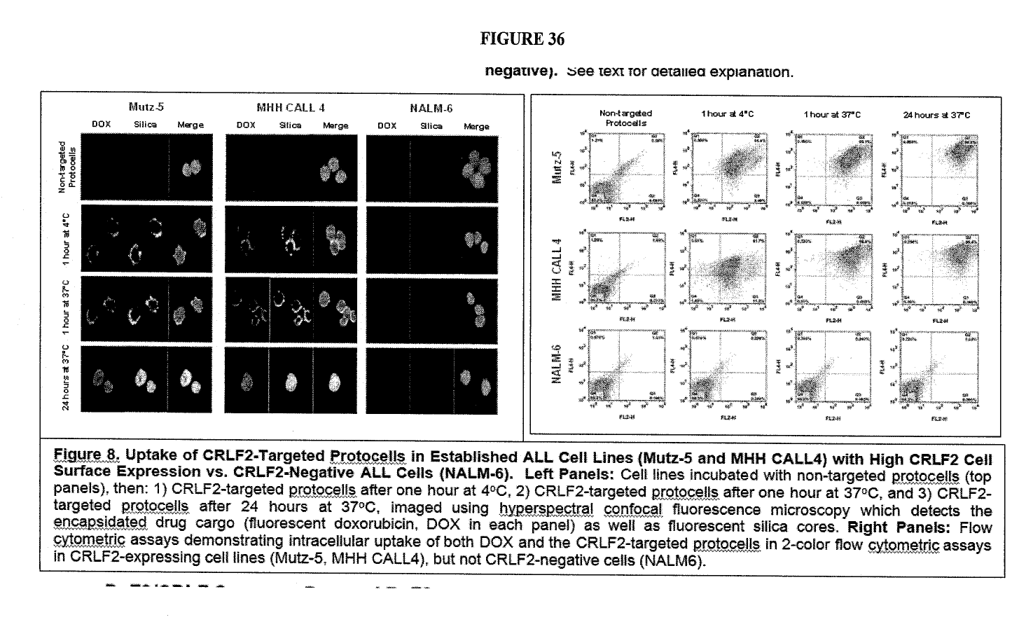

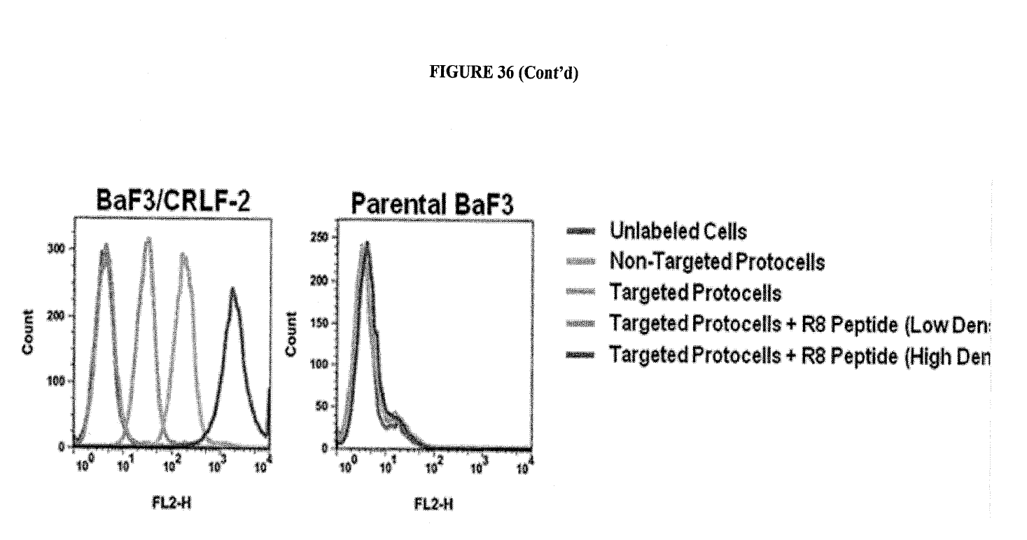

The CRLF2-targeting peptide shown in FIG. 31 was conjugated to protocells. CRLF2-targeted protocells were demonstrated to possess a 1,000-fold higher affinity for engineered BaF3-CRLF2 cells expressing high levels of CRLF2 (FIG. 35), and for the MUTZ5 or MHHCALL4 cells (established human ALL cells lines with high CRLF2 expression and JAK kinase mutations) (FIG. 36), when compared to untargeted protocells, the parental BAF3 cell line, or the CRLF2-negative NALM6 ALL cell line which served as controls (FIGS. 35 and 36). This affinity was also achievable at very low peptide densities due to the fluid protocell surface, potentially minimizing non-specific binding and/or immune responses. Targeted protocells loaded with doxorubicin (which is intrinsically fluorescent) were able to selectively bind to cells expressing CRLF2, and after incubation at 37° C., to become internalized and deliver drug to the cytoplasm of the cells within 24 hours while showing no non-specific interactions with control cells (FIG. 35B, FIG. 36). Further, modification of the protocell surface with an octa-arginine (R8) peptide promoted this selective internalization in a density-dependent manner, proving that protocells support complex synergistic interactions enabling targeting and internalization for cancers whose targeting peptides might poorly internalized (FIG. 35C).

We are excited that these preliminary studies demonstrate that we can selectively target CRLF2-expressing ALL cells with CRLF2-targeted nanocarriers, that the protocell and its drug cargo is internalized, and taken up by the cytoplasm. An additional series of studies will investigate the added benefit of conjugating multiple targeting peptides to the surface of a single particle. We postulate that protocells that recognize both CD 19 and CD22 might have an improved therapeutic index compared to those that target a single antigen, and might limit the potential emergence of resistant lines that can arise by loss of a single surface protein.

### Defining Optimal Chemotherapeutic Cargoes and Determining Therapeutic Efficacy of ALL-Targeted Protocells In Vitro.

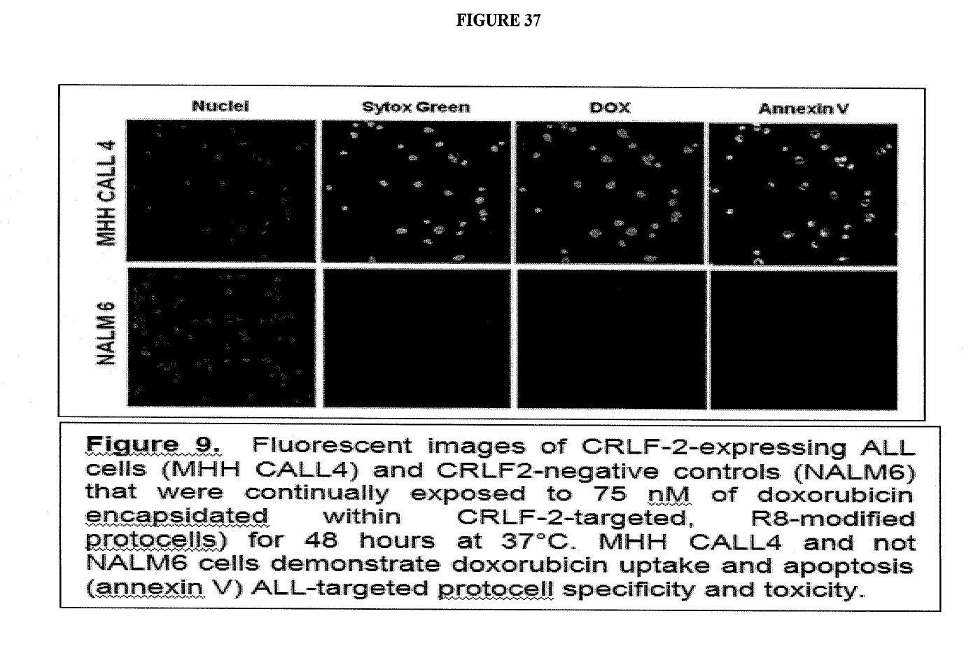

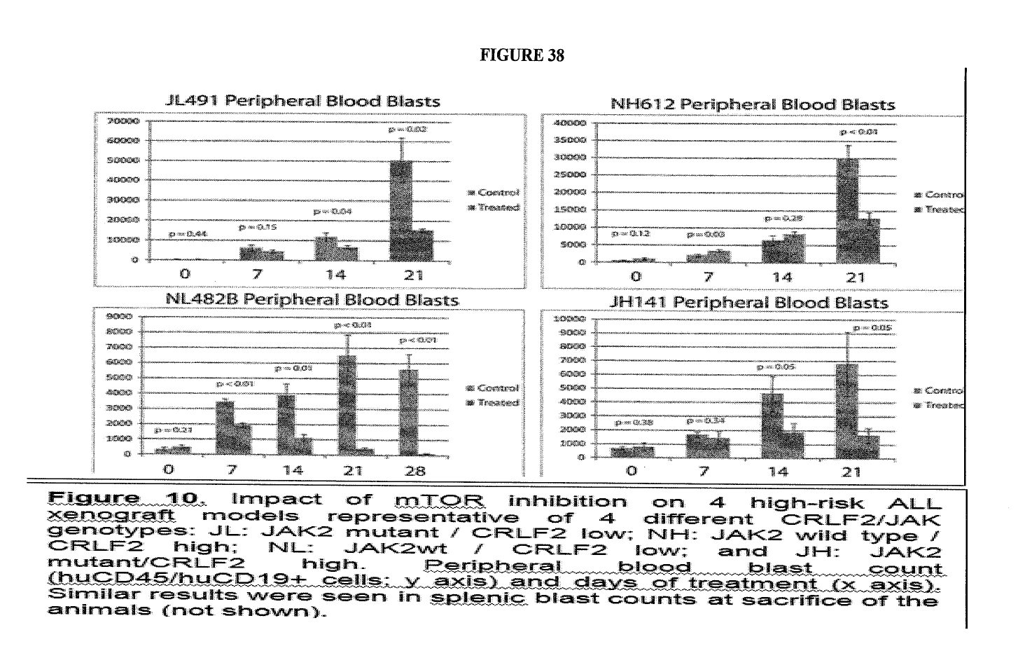

The ability of protocells to protect their therapeutic cargo until released within the target cell and to deliver multiple cargoes are being exploited to determine the most efficacious drug combinations for packaging into ALL-targeted protocells. Using the in vitro ALL cell line models described above in Aim 1b), we are testing traditional ALL therapeutic drug combinations (FIG. 37) as well as novel compounds that demonstrate efficacy against resistant forms of high-risk ALL (such as the mTOR pathway inhibitor sirolimus) that we have identified in high throughput screens or ALL xenograft models (FIG. 38). The therapeutic efficacy of drugs encapsidated in protocells is being compared to exposure of the cell lines to free drug(s) using cell biologic, flow cytometric, and phosphoflow cytometric assays, allowing us to test and model pharmacodynamic assessments of target inhibition in ALL cells in vitro.

When CRLF2-targeted protocells with encapsidated doxorubicin were incubated with the CRLF2-expressing cell line MHHCALL4, binding, protocell and drug uptake, and doxorubicin release into the cytoplasm could be demonstrated in CRLF2-expressing cells but not in control cells. Although CRLF2-expressing high-risk ALL patients tend to be resistant to intensive therapeutic regimens,⁹⁻¹¹ we demonstrated in very preliminary studies that after uptake and drug delivery, CRLF2-targeted protocells with encapsidated doxorubicin promoted rapid apoptosis and cell death in MHH CALL4 cells (FIG. 37) In order to validate compounds and ALL-targeted/drug-loaded protocell designs as being cytotoxic to ALL cells, and to choose those compounds which are most active in vitro (Aim 1c) for further screening in vivo in xenograft models (Aim 2), it is necessary to assess response and targeting behavior in human ALL cell lines. This is an approach that our team of investigators have used successfully to prioritize compounds for our ALL xenograft experiments, leading to our current trials of mTOR inhibitors in ALL. In Aim 1c), we will validate the cytotoxic activity of mTOR inhibitors in high-risk ALL cases (using cell lines reflective of the CRLF2/JAK genotype/phenotype), based on preliminary xenograft data that demonstrate activity of this class of drugs in this subset of leukemias. All lines used will include CD19+ and CD19− or CD22+ and CD22− variants of T cell lines, and CRLF2+ and CRLF2− variants of the same line (NALM6 is CRLF2-negative and we have engineered a CRLF2+NALM-6 line marked with GFP and Click Beetle Green luciferase figures s3 and s4, below)).

Our panel will also include the high CRLF2-expressing/JAK mutated MUTZ5 and MHHCALL4 human ALL cell lines which are markedly drug resistant and that express both CD19 and CD22. In addition a variety of ALL cell lines that express CD19 and/or CD22 will be used in cytotoxicity experiments. The development of reagents that recognize antigens present on a very large proportion of B-cell leukemias and lymphomas will dramatically increase the scope of our previous work that targeted a very narrow cohort of pediatric ALLs expressing CRLF2. These studies will include comprehensive dose-response curves using the agents alone and in combination with drugs used in standard protocols, as we have previously published. The end points will be growth assays as well as biochemical and flow cytometric measurements of apoptosis/necrosis measured at a number time points to determine both early and late effects. Preliminary experiments have validated the cytotoxic efficacy of some of these compounds, although their potency appears low in some cases. If this continues to be the case, we can make a compelling argument for protocell based delivery that results in elevated intracellular concentrations compared to what can be achieved following systemic administration. Each of the protocell variants developed in Aims 1a) and 1b) will be tested: free compound, compound loaded into protocells, and targeted nanocarriers against CD 19 and CD22 using either single chain antibodies or targeting peptides. If experiments in Aim 1a) and 1b) show that internalization is improved using an anti-CD22 targeting agent (as CD22 is known to be rapidly internalized after antibody binding), then we will test that construct as well. The goal of Aim 1c) is to find the most cytotoxic combinations of targeting ligand, cargo, and protocell to test in vivo in ALL xenograft models.

### Example 10

### Model CRLF2 and CD99+ Cell Lines for Selection and In Vitro and In Vivo Studies

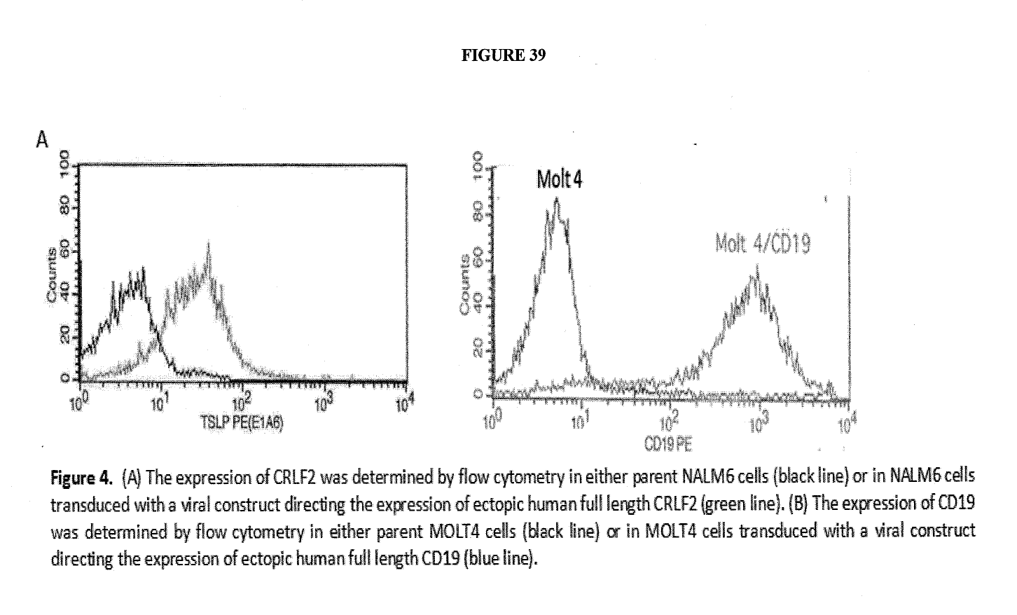

Fluorescently tagged NALM6 cells were transduced with a retrovirus directing the expression of ectopic human CRLF2 and stable clones with a 10-fold increase in surface expression (FIG. 39A) were established. This increase in expression is adequate for initial experiments and these cells have been used in preliminary studies using chick embryos. In addition, these cells will be evaluated for their ability to form xenografts with characteristics similar to those of the parent cells. We also constructed similar viral constructs encoding human CD99 as proposed in the last progress report and established a series of fluorescent NALM6 clones that with very high levels of ectopic gene expression. However, we failed to detect trafficking of CD99 to the cell surface based on a flow analysis. An alternative cloning strategy to allow for proper CD99 trafficking and increased relative levels of surface CRLF2 in the cell lines that will be used in the in vivo assays is now being used.

A sequence listing of all sequences disclosed in the present application follows:

Sequence Listings of Peptides/Nucleotides:
SEQ ID NO: 1
MTAAPVHGGHHHHHH
SEQ ID NO: 2
RRRRRRRRGGC
SEQ ID NO: 3
GLFHAIAHFIHGGWHGLIHGWYGGGC
SEQ ID NO: 4
MTAAPVH
SEQ ID NO: 5
LTTPNWV
SEQ ID NO: 6
AAQTSTP
SEQ ID NO: 7
TDAHASV
SEQ ID NO: 8
FSYLPSH
SEQ ID NO: 9
YTTQSWQ
SEQ ID NO: 10
MHAPPFY
SEQ ID NO: 11
AATLFPL
SEQ ID NO: 12
LTSRPTL
SEQ ID NO: 13
ETKAWWL
SEQ ID NO: 14
HWGMWSY
SEQ ID NO: 15
SQIFGNK
SEQ ID NO: 16
SQAFVLV
SEQ ID NO: 17
WPTRPWH
SEQ ID NO: 18
WVHPPKV
SEQ ID NO: 19
TMCIYCT
SEQ ID NO: 20
ASRIVTS
SEQ ID NO: 21
WTGSYRW
SEQ ID NO: 22
NILSLSM
SEQ ID NO: 23
RRRRRRRR
SEQ ID NO: 24
GLFHAIAHFIHGGWHGLIHGWY
SEQ ID NO: 25
WPTXPW[—H]
SEQ ID NO: 26
---S[FW][ST]XWXX--WX------
SEQ ID NO: 27
-----XSPXXWXXXXX-------
SEQ ID NO: 28
GNQSSNFGPMKGGNFGGRSSGPYGGGGQYFAKPRNQGGY
SEQ ID NO: 29
RRMKWKK
SEQ ID NO: 30
PKKKRKV
SEQ ID NO: 31
KR[PAATKKAGQA]KKKK
SEQ ID NO: 32
acatgaggat tacccatgt
SEQ ID NO: 33
acatgaggat cacccatgt
SEQ ID NO: 34
FS--YLP[—S][—H]
SEQ ID NO: 35
MT-AAP[VFW]H

1. A CRLF-2 binding peptide consisting essentially of a peptide according to the sequence MTAAPVH (SEQ ID NO: 4), LTTPNWV (SEQ ID NO:5), AAQTSTP (SEQ ID NO:6), TDAHASV (SEQ ID NO:7), FSYLPSH (SEQ ID NO: 8), YTTQSWQ (SEQ ID NO:9), MHAPPFY (SEQ ID NO:10), AATLFPL (SEQ ID NO:11), LTSRPTL (SEQ ID NO:12), ETKAWWL (SEQ ID NO:13) HWGMWSY (SEQ ID NO:14), SQIFGNK (SEQ ID NO:15), SQAFVLV (SEQ ID NO:16), WPTRPWH (SEQ ID NO:17), WVHPPKV (SEQ ID NO:18), TMCIYCT (SEQ ID NO:19), ASRIVTS (SEQ ID NO:20), WTGSYRW (SEQ ID NO:21) or NILSLSM (SEQ ID NO:22).

2. (canceled)

3. (canceled)

4. (canceled)

5. (canceled)

6. A cell-targeting porous protocell comprising:
a nanoporous silica or metal oxide core with a supported lipid bilayer and at least one further component selected from the group consisting of
a CRLF-2 binding peptide according to claim 1 which is covalently linked or complexed to the surface of said protocell;
a fusogenic peptide that promotes endosomal escape of protocells and encapsulated DNA, and
at least one additional cargo component selected from the group consisting of double stranded linear DNA;
plasmid DNA;
an anticancer drug;
an imaging agent,
small interfering RNA, small hairpin RNA, microRNA, or a mixture thereof,
wherein one of said cargo components is optionally conjugated further with a nuclear localization sequence.

7. The protocell according to claim 6 wherein said additional cargo component is an anti-cancer drug and said lipid bilayer is fused to said nanoporous core.

8. The protocell according to claim 7 wherein said anticancer drug is selected from the group consisting of doxorubicin, 5-fluorouracil, cisplatin, cyclophosphamide, vincristin (oncovin), vinblastine, prednisolone, procarbazine, L-asparaginase, cytarabine, hydroxyurea, 6-mercaptopurine, methotrexate, 6-thioguanine, bleomycin, etoposide, ifosfamide and mixtures thereof.

9. (canceled)

10. The protocell according to claim 6 wherein said fusogenic protein consists essentially of H5WYG peptide (SEQ ID NO: 24) or an eight mer of polyarginine (SEQ ID NO: 23).

11. (canceled)

12. The protocell according to claim 6 comprising plasmid DNA, wherein said plasmid DNA is optionally modified to express a nuclear localization sequence.

13. The protocell according to claim 12 wherein said plasmid DNA is supercoiled or packaged plasmid DNA

14. (canceled)

15. The protocell according to claim 12 wherein said plasmid DNA is modified to express a nuclear localization sequence.

16. The protocell according to claim 12 wherein said DNA is histone-packaged supercoiled plasmid DNA comprises a mixture of human histone proteins.

17. (canceled)

18. (canceled)

19. The protocell according to claim 6 wherein said plasmid DNA is capable of expressing a polypeptide toxin, a small hairpin RNA (shRNA) or a small interfering RNA (siRNA).

20. The protocell according to claim 19 wherein said polypeptide toxin is selected from the group consisting of ricin toxin A chain, diphtheria toxin A chain or cholera toxin A chain.

21. (canceled)

22. (canceled)

23. (canceled)

24. The protocell according to claim 6 wherein said nuclear localization sequence is a peptide according to SEQ ID NO: 28, SEQ ID NO: 29, SEQ ID NO: 30 or SEQ ID NO: 31.

25. (canceled)

26. A CRLF-2 and/or CD-19 targeting protocell comprising:
(a) a core comprising a plurality of negatively-charged, nanoporous, nanoparticulate silica cores that are optionally modified with an amine-containing silane and that are interspersed with one or more anticancer agents that are useful in the treatment of a cancer that overexpresses CRLF-2 and/or CD-19; and
(b) a lipid bilayer which encapsulates the core and which comprises one of more lipids selected from the group consisting of 1,2-dioleoyl-sn-glycero-3-phosphocholine (DOPC), 1,2-dipalmitoyl-sn-glycero-3-phosphocholine (DPPC), dioleylglycero triethyleneglycyl iminodiacetic acid (DOIDA), distearylglycerotriethyleneglycyl iminodiacetic acid (DOIDA), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,
wherein the lipid bilayer comprises a cationic lipid and one or more zwitterionic phospholipids and contains on its surface at least one peptide that targets CRLF-2 and/or CD19.

27. The protocell of claim 26, wherein said peptide that targets CRLF-2 is a CRLF-2 binding peptide consisting essentially of a peptide sequence according to claim 1.

28. (canceled)

29. (canceled)

30. (canceled)

31. (canceled)

32. (canceled)

33. (canceled)

34. A CRLF-2 and/or CD 19-targeting protocell comprising:
(a) a core comprising a plurality of negatively-charged, nanoporous, nanoparticulate silica cores that are optionally modified with an amine-containing silane such as N-(2-aminoethyl)-3-aminopropyltrimethoxysilane (AEPTMS) and that are interspersed with one or more siRNA that are useful in the treatment of ALL; and
(b) a lipid bilayer which encapsulates the core and which comprises one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,
wherein the lipid bilayer comprises a cationic lipid and one or more zwitterionic phospholipids and contains on its surface at least one peptide that targets CRLF-2 and/or CD 19.

35. (canceled)

36. (canceled)

37. (canceled)

38. (canceled)

39. A pharmaceutical composition comprising a population of protocells according to claim 1 and a pharmaceutically-acceptable carrier, additive or excipient.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A method of treating a subject suffering from a cancer that overexpresses CLRF-2 acute lymphoblastic leukemia (ALL), the method comprising administering to the subject a pharmaceutically-effective amount of a population of protocells according to claim 6 and, optionally, an additional anti-cancer agent.

58. The method according to claim 57 wherein said cancer is acute lymphoblastic leukemia.

59. (canceled)

60. (canceled)

61. A protocell nanostructure comprising:
(a) a porous nanoparticle comprising a plurality of pores;
(b) at least one lipid bilayer surrounding the porous particle to form a protocell;
(c) at least one CRLF-2 targeting peptide according to claim 1 conjugated to said lipid bilayer; and
(d) a cargo component which comprises at least one therapeutic agent loaded into the protocell nanostructure for delivery to a patient.

62. The protocell of claim 61, wherein said cargo component includes at least one component is selected form the group consisting of small molecules, ShRNA, siRNa or a polypeptide toxin.

63. (canceled)

64. (canceled)

65. The protocell according to claim 61, wherein said therapeutic agent is an anticancer agent selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof

66. (canceled)

67. (canceled)

68. (canceled)

69. (canceled)

70. (canceled)

71. The protocell according to claim 61, wherein said targeting peptide is any of the CRLF-2 targeting peptides as set forth in attached FIGS. 10-14 hereof.

72. The protocell according to claim 61, wherein said targeting peptide is a consensus sequence according to SEQ ID NO:25, SEQ ID NO:26, SEQ ID NO:27, SEQ ID NO:34 or SEQ ID NO:35.

73. A CRLF-2 binding peptide sequence as set forth in any of FIGS. 10-14 hereof.

74. (canceled)

75. (canceled)

76. (canceled)

77. (canceled)
